(19) United States
(12) Patent Application Publication
(10) Pub. No.: US 2026/0001917 A1
(43) Pub. Date: Jan. 1, 2026

(54) MODIFIED ADENO-ASSOCIATED VIRUS CAPSID PROTEINS AND METHODS THEREOF

(71) Applicant: MOUNT SPEC INVESTMENTS PTY LTD, Queensland (AU)

(72) Inventors: Christopher LAYTON, Fortitude Valley (AU); Sergei KOZLOV, Fortitude Valley (AU); Jason STEEL, AA (AU)

(21) Appl. No.: 18/868,231

(22) PCT Filed: May 31, 2023
(86) PCT No.: PCT/AU2023/050463

§ 371 Date: Nov. 22, 2024

(30) Foreign Application Priority Data
May 31, 2022 (AU) 2022901483

(51) Int. Cl. C07K 14/005; A61K 48/00; C12N 15/86

(52) U.S. Cl. CPC C07K 14/005; A61K 48/0075; C12N 15/86; C12N 2750/14122; C12N 2750/14143

(57) ABSTRACT

The present invention relates to modified recombinant adeno-associated virus (AAV) capsid proteins and AAV particles thereof. In one aspect, the modified recombinant AAV particles provide for increased transduction of retinal cells when compared to the effect of a recombinant AAV particle that does not comprise the modification. The present invention also relates to nucleic acids encoding the modified AAV capsid proteins and AAV particles thereof.

### CROSS REFERENCE TO RELATED APPLICATIONS

This application is a U.S. Nat'l Phase of Int'l Appl. No. PCT/AU2023/050463, filed May 31, 2023, which claims priority to Int'l AU application No. 2022901483, filed May 31, 2022, each of which is incorporated herein in its entirety and for all purposes.

### SEQUENCE LISTING DISCLOSURE

The contents of the electronic sequence listing (29532200001601.xml; Size: 193,257 bytes; and Date of Creation: Nov. 11, 2024) is herein incorporated by reference in its entirety.

### FIELD OF THE INVENTION

The present invention relates to modified recombinant adeno-associated virus (AAV) capsid proteins and AAV particles thereof. In one aspect, the modified recombinant AAV particles provide for increased transduction of retinal cells when compared to the effect of a recombinant AAV particle that does not comprise the modification. The present invention also relates to nucleic acids encoding the modified AAV capsid proteins and AAV particles thereof.

### BACKGROUND OF THE INVENTION

Recombinant adeno-associated virus (AAV) vectors are an effective means of delivering gene therapy. Use of such recombinant AAV vectors has steadily increased over recent years due to (1) their ability to infect a variety of dividing and non-dividing cells, (2) their tendency to remain episomal rather than integrating into target cell genomes, (3) their capability to effect long-term expression of therapeutic transgenes, (4) their lower likelihood of eliciting an immune response compared with other viral vectors, (5) their inability to replicate and initiate productive infection without the assistance of a helper virus, such as adenovirus or herpes simplex virus, and (6) their lack of association with any known disease (Wang et al., 2019).

Recombinant AAV vector design is based on the genomic engineering of wild type AAVs (Agbandje-McKenna, M. & Kleinschmidt 2011; Samilski et al., 2014). AAVs have a single-stranded DNA genome that is approximately 4.7 kb long and encodes 5′ and 3′ inverted terminal repeat sequences (containing the cis-acting elements for replication and packaging), a rep gene and a cap gene. The rep gene is transcribed and alternatively spliced to produce four different proteins responsible for genome replication and packaging as well as transcriptional regulation. The cap gene is transcribed from a single promoter, has two translation start sites and is alternatively spliced to produce three structural proteins (VP1, VP2 and VP3). These assemble to form an icosahedral capsid consisting of 50 units of VP3 and 10 units each of VP1 and VP2. Within the 5′ end of the cap gene, and out of frame with the cap open reading frame, is the AAP gene. This encodes the assembly activating protein which helps ensure efficient virus assembly. The cap gene effects virus stability and tropism for particular tissues.

Depending on the target tissue or cell type, a given naturally occurring AAV vector serotype may be relatively ineffective at transducing the target tissue or cell. One such example is the transduction of retinal cells (e.g., retinal pigment epithelium and photoreceptors). Efforts have therefore been made to engineer the AAV capsid protein. For instance, individual amino acids of the capsid have been engineered to enhance virus stability and to improve viral tropism. In one example, conserved tyrosine residues have been substituted with phenylalanine residues to avoid tyrosine phosphorylation and subsequent ubiquitin-mediated proteasomal degradation (Zhong et al., 2008). Similarly, conserved serine and threonine amino acids have been substituted with valine residues (Aslanidi et al., 2012; Aslanidi et al., 2013) and conserved lysines substituted with glutamic acid (Li et al., 2015) also to prolong AAV longevity. Whilst recombinant AAV capsids with improved stability and enhanced tropism have been generated for some target cells or tissues, issues such as neutralising antibodies towards AAVs (Halbert et al., 2006) are still highly prevalent. For example, neutralising antibodies towards AAV2 variant 7m8 have been reported previously (Dalkara et al., 2013).

Moreover, with respect to transduction of retinal cells of the eye, efficient transduction of the human retina remains a major challenge (Bordet et al., 2019). One example is the transduction of the human retina via delivery into the vitreous. This route of administration is preferable over other routes because it is simpler and safer whilst providing a greater volume of diffusion. However, the presence of AAV neutralising antibodies in the vitreous, the physical barrier created by the inner limiting membrane of the retina and utilising an AAV with suitable tropism for photoreceptor cells or the retinal pigment epithelium are major hurdles in relation to the administration of gene therapy to the eye.

In view of the above described limitations, there is a need for recombinant adeno-associated viruses (AAVs) that have improved transduction efficiency and/or are associated with a reduced immunogenic response.

### SUMMARY OF THE INVENTION

The present inventors demonstrate for the first time the generation of recombinant adeno-associated viruses (AAV) with modified capsids that provide for increased transduction efficiency and reduced immunogenicity when compared to AAVs not having the capsid modifications. Advantageously, these AAVs, when packaged with an expression cassette, have improved therapeutic application for diseases, conditions or disorders requiring gene therapy, including for the treatment of ocular disorders such as age-related macular degeneration.

In a first aspect of the invention, there is therefore provided a recombinant adeno-associated virus (AAV) capsid protein comprising an insertion according to the sequence set forth in SEQ ID NO: 1 or functional equivalents thereof.

In another aspect of the invention, there is provided a recombinant adeno-associated virus (AAV) VP capsid protein comprising an insertion according to the sequence set forth in SEQ ID NO: 1 or functional equivalents thereof, wherein the insertion is in one or more or all of the VP1, VP2 and VP3 capsid proteins, preferably the VP3 capsid protein, optionally comprising a sequence set forth in SEQ ID NOs: 130-133 or 139-140.

In an embodiment, the insertion is in loop IV, Region 6 or variable region VIII (VR-VIII) of an AAV8 capsid protein or in a corresponding position of a capsid protein of an AAV serotype other than AAV8. In this embodiment, the insertion is relative to a parental AAV capsid protein.

In an embodiment, the insertion is located at a position corresponding to amino acid 590, preferably of the capsid protein of AAV8 or in a corresponding position of a capsid protein of an AAV serotype other than AAV8.

In an embodiment, the insertion is relative to a parental AAV capsid protein according to the sequence set forth in SEQ ID NO: 121 or 125.

In an embodiment, the insertion comprises a sequence according to any one of the sequences set forth in SEQ ID NO:2-108.

In an embodiment, the insertion has at least 60% sequence identity, at least 70% sequence identity, at least 80% sequence identity, at least 90% sequence identity or 100% sequence identity with the sequence according to the sequence set forth in SEQ ID NO:1. In another embodiment, the insertion comprises a sequence that differs by no more than 1 or 2 amino acids when compared with the sequence according to the sequence set forth in SEQ ID NO:1.

In an embodiment, the insertion comprises a sequence according to the sequence set forth in SEQ ID NO:109 or SEQ ID NO:110.

In an embodiment, the insertion comprises a linker flanking the 5′ and/or 3′ end of the insertion. Optionally, the linker comprises a sequence according to SEQ ID NO:111 (“TG”; e.g., encoded by ACCGGT) and/or SEQ ID NO:112 (“GLS”; e.g., encoded by GTCTTTCGA). In an embodiment, the insertion comprises the sequence SEQ ID NO: 129 and/or 138.

In an embodiment, the insertion is in one or more or all of the VP1, VP2 and VP3 capsid proteins, preferably the VP3 capsid protein, optionally comprising a sequence set forth in SEQ ID NOs: 130-133 or 139-140.

In an embodiment, the recombinant capsid protein comprises a mutation selected from one or more or all of Y447F, T494V and Y733F. Preferably, the recombinant capsid protein comprises each of Y447F, T494V and Y733F in the AAV8 capsid protein, optionally comprising a sequence set forth in any one of SEQ ID NOs: 135 or 142.

In an embodiment, the recombinant AAV capsid protein is selected from the group consisting of a recombinant AAV2, AAV4, AAV7 or AAV10 capsid protein.

In an aspect of the invention, there is provided a recombinant AAV particle comprising an AAV capsid protein described herein. Preferably, the AAV particle is packaged in the presence of an AAV2 rep gene encoding an AAV2 rep protein, optionally as defined according to the sequence set forth in SEQ ID NO: 123.

In an aspect of the invention, there is provided an isolated nucleic acid encoding a recombinant adeno-associated virus (AAV) capsid protein described herein, optionally comprising a sequence set forth in SEQ ID NO: 134 or 141.

In an aspect of the invention, there is provided an isolated nucleic acid encoding a recombinant adeno-associated virus (AAV) capsid protein having at least 60% sequence identity, at least 70% sequence identity, at least 80% sequence identity, at least 90% sequence identity or 100% sequence identity to the sequence set forth in SEQ ID NO: 134 or 141.

In an aspect of the invention, there is provided an isolated nucleic acid encoding a recombinant adeno-associated virus (AAV) particle described herein, optionally comprising a sequence set forth in SEQ ID NO:136 or 143.

In an aspect of the invention, there is provided an isolated nucleic acid encoding a recombinant adeno-associated virus (AAV) particle having at least 60% sequence identity, at least 70% sequence identity, at least 80% sequence identity, at least 90% sequence identity or 100% sequence identity to the sequence set forth in SEQ ID NO:136 or 143.

In an aspect of the invention, there is provided an isolated nucleic acid encoding a recombinant adeno-associated virus (AAV) capsid protein having at least 60% sequence identity, at least 70% sequence identity, at least 80% sequence identity, at least 90% sequence identity or 100% sequence identity to the sequence set forth in SEQ ID NO: 124.

In an aspect of the invention, there is provided an isolated nucleic acid encoding a recombinant adeno-associated virus (AAV) capsid VP3 protein, wherein the VP3 protein has at least 60% sequence identity, at least 70% sequence identity, at least 80% sequence identity, at least 90% sequence identity or 100% sequence identity to the sequence set forth in SEQ ID NOs: 130-133 or 139-140.

In an embodiment, the nucleic acid encoding a recombinant AAV capsid protein comprises a p5 enhancer element, preferably an AAV2 p5 enhancer element. Preferably, the p5 enhancer element is located downstream of the AAV capsid gene. Optionally, the nucleic acid encoding a recombinant adeno-associated virus (AAV) capsid protein comprises an AAV8 3′UTR.

In an embodiment, the nucleic acid encoding a recombinant AAV particle comprises an AAV2 rep nucleic acid sequence optionally as defined according to the sequence set forth in SEQ ID NO: 122. Optionally expression of the AAV2 rep nucleic acid sequence is driven by a p5 promoter.

In an embodiment, the AAV2 rep gene comprises a mutation ATG to ACG in the start codon of the AAV2 rep gene so that translation begins at a downstream, in-frame methionine, optionally according to the sequence defined according to SEQ ID NO: 114.

In an embodiment, the nucleic acid encoding a recombinant AAV particle comprises AAV2 inverted terminal repeats (ITRs).

In an embodiment, the recombinant AAV particle comprises an expression cassette for expressing a therapeutic molecule. In a further embodiment, the therapeutic molecule is DNA, mRNA, CRNA, and cDNA, RNA, siRNA, shRNA and hpRNA. In a preferred embodiment, the therapeutic molecule is suitable for treating an ocular disease, optionally selected from the group consisting of retinitis pigmentosa, diabetic retinopathy, cystoid macular oedema, clinically significant macular oedema, uveitis, iritis, giant cell arteritis, vasculitis, pars planitis, corneal transplant rejection, intraocular inflammation or lamellar corneal transplant rejection, macular degeneration, central retinal vein occlusion, branch retinal vein occlusion and ocular neovascularisation.

In an embodiment, the therapeutic molecule inhibits angiogenesis and/or inflammation. In a further embodiment, the therapeutic molecule:
comprises endostatin, angiostatin, or a fusion of endostatin and angiostatin;
is a binding protein;
comprises an antigen binding site of an antibody;
is selected from the group consisting of is ranibizumab, bevacizumab, and aflibercept;
inhibits inflammation; or,
is interleukin 10 (IL-10), interleukin 1 receptor antagonist (IL-1RA); or a fusion of IL-10 and IL-1RA.

In a further embodiment, the therapeutic molecule is an inhibitor of vascular endothelial growth factor (VEGF), placental growth factor (PIGF), platelet derived growth factor (PDGF), fibroblast growth factor (FGF), TIE ligands (Angiopoietins), ephrins, angiopoietin-like 3 (ANGPTL3), angiopoietin-like 4 (ANGPTL4), insulin-like growth factor-I (IGF-I), epidermal growth factor (EGF), connective tissue growth factor (CTGF), transforming growth factor alpha (TGF-α), transforming growth factor beta (TGF-β) or TNF-alpha (e.g., anti-inflammatory soluble TNF-R). In a preferred embodiment, the therapeutic molecule is an inhibitor of vascular endothelial growth factor (VEGF), optionally selected from a VEGF antibody, VEGF receptor antibody or VEGF siRNA.

In a further embodiment, the expression cassette comprises a retinal-specific promoter for driving expression of the therapeutic molecule, optionally selected from the group consisting of rhodopsin, rhodopsin kinase, RPE65 and retinaldehyde binding protein 1 (RLBP1) or any of the promoters listed in Table 5 herein.

In an embodiment, the expression cassette comprises:
a) a kill switch comprising a first site-specific recombination sequence and a second site-specific recombination sequence;
b) a regulatable element operably linked to a nucleic acid sequence encoding a therapeutic molecule, wherein activity of the regulatable element is regulated by a regulator compound; and
c) a constitutive promoter operably linked to a nucleic acid sequence encoding a regulator compound-binding polypeptide which is capable of binding a regulator compound, wherein upon binding the regulator compound, the regulator compound-binding polypeptide regulates expression of the therapeutic molecule, wherein activation of the kill switch by recombination between the first site-specific recombination sequence and the second site-specific recombination sequence silences expression of the nucleic acid encoding the therapeutic molecule from the cassette.

In an aspect of the invention, there is provided a pharmaceutical composition comprising an AAV particle described herein and one or more carriers or excipients.

In an aspect of the invention, there is provided a method for increasing transduction efficiency of an adeno-associated virus (AAV) particle within a target cell, tissue or organ, the method comprising contacting the target cell, tissue or organ with an AAV particle described herein under conditions sufficient for transduction of the AAV particle within the target cell, tissue or organ, wherein transduction efficiency is increased when compared to an AAV particle not having the capsid insertion.

In a preferred embodiment, the AAV particle comprises an expression cassette for expressing a therapeutic molecule. In this embodiment, the expression of the therapeutic molecule is increased when compared to an AAV particle comprising an expression cassette and not having the capsid insertion.

In this embodiment, the method is an ex vivo, in vitro or in vivo method. In a further embodiment, the target cell is a retinal cell, the target tissue is retinal tissue and the target organ is the eye.

In an embodiment, transduction efficiency is increased by at least 5, 10, 15, 20, 25, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400% or more when compared to an AAV particle not having the capsid insertion.

In another embodiment, transduction efficiency is increased by at least 1.5, 2, 3, 4, 5, 6, 7, 8, 9, 10, 15, 20, 25, 30, 35, 40, 45, 50 fold or more when compared to an AAV particle not having the capsid insertion.

In an aspect of the invention, there is provided a method for reducing an immune response to an adeno-associated virus (AAV) particle in a target cell, tissue or organ, the method comprising contacting the target cell, tissue or organ with an AAV particle described herein under conditions sufficient for transduction of the AAV particle in the target cell, tissue or organ, wherein the immune response is reduced when compared to an AAV particle not having the capsid insertion.

In an embodiment, the target cell is a retinal cell, the target tissue is retinal tissue and the target organ is the eye.

In an aspect of the invention, there is provided a method for treating a condition, disorder or disease in a subject in need thereof, comprising administering an AAV particle or composition thereof described herein to the subject, thereby treating the condition, disorder or disease in the subject.

In an aspect of the invention, there is provided use of an AAV particle or composition thereof described herein in the preparation of a medicament for treating a condition, disorder or disease in a subject in need thereof.

In an aspect of the invention, there is provided an AAV particle or composition thereof described herein for use in treating a condition, disorder or disease in a subject in need thereof.

In an embodiment, the condition, disorder or disease is an ocular disease, optionally selected from the group consisting of retinitis pigmentosa, diabetic retinopathy, cystoid macular oedema, clinically significant macular oedema, uveitis, iritis, giant cell arteritis, vasculitis, pars planitis, corneal transplant rejection, intraocular inflammation or lamellar corneal transplant rejection, macular degeneration, central retinal vein occlusion, branch retinal vein occlusion and ocular neovascularisation.

In an embodiment, the subject has at least one symptom of an ocular disorder selected from the group comprising decreased peripheral vision, decreased central vision, decreased night vision and loss of colour perception.

In an embodiment, the AAV particle or composition thereof to be administered to a subject in need thereof comprises a modified capsid protein described herein and an expression cassette comprising:
a) a kill switch comprising a first site-specific recombination sequence and a second site-specific recombination sequence;
b) a regulatable element operably linked to a nucleic acid sequence encoding a therapeutic molecule, wherein activity of the regulatable element is regulated by a regulator compound; and
c) a constitutive promoter operably linked to a nucleic acid sequence encoding a regulator compound-binding polypeptide which is capable of binding a regulator compound, wherein upon binding the regulator compound, the regulator compound-binding polypeptide regulates expression of the therapeutic molecule, wherein activation of the kill switch by recombination between the first site-specific recombination sequence and the second site-specific recombination sequence silences expression of the nucleic acid encoding the therapeutic molecule from the cassette.

In this embodiment, the AAV particle may be administered intravitreally or subretinally and the kill switch may be activated by administering a site-specific recombinase or a nucleic acid encoding a site-specific recombinase, wherein the site-specific recombinase catalyses the recombination between a first site-specific recombination sequence and a second site-specific recombination sequence, thereby silencing expression of the therapeutic molecule. In a further embodiment, the treatment comprises administering the regulator compound to the eye topically or in eye drops.

In an embodiment, a method or use described herein comprises administration of an immunosuppressant. Where the administration of immunosuppressant is contemplated, the immunosuppressant is preferably administered prior to the administration of the recombinant AAV or composition thereof or alternatively, concurrently with the administration of the recombinant AAV or composition thereof. Preferably, the recombinant AAV or composition thereof is administered once, however if an immunosuppressant is administered, the recombinant AAV or composition thereof may be administered more than once.

In a further embodiment, a method or use described herein comprises administration of an additional therapy, optionally selected from the group comprising surgery, lens replacement with an intraocular lens, laser surgery or medication. Any additional therapeutic treatment, including the administration of immunosuppressant, may be administered once, twice, three times or more to achieve the desired therapeutic effect.

In an embodiment, a recombinant AAV particle, or composition thereof defined herein is administered intravitreally or subretinally. In an embodiment, the additional therapy and/or immunosuppressant is administered intravenously, orally, subcutaneously or intramuscularly.

In an aspect of the invention, there is provided an isolated mammalian cell comprising a recombinant adeno-associated virus (AAV) particle described herein. In an embodiment, the mammalian cell is a human cell, preferably a human retinal cell, optionally selected from the group consisting of photoreceptors, retinal ganglion cells, bipolar cells, trabecular meshwork, retinal pigment epithelium cells, amacrine cells, astrocytes, horizontal cells, microglia or Muller glia cells.

In an aspect of the invention, there is provided a kit comprising a recombinant adeno-associated viral (AAV) particle described herein or a composition thereof for preventing or treating, or when used for preventing or treating, a disease, condition or disorder in a subject. Optionally, the kit may comprise:
a recombinase or an expression cassette comprising a nucleic acid sequence encoding a recombinase;
an eye drop formulation or topical formulation;
a recombinase or an expression cassette comprising a nucleic acid sequence encoding a recombinase for silencing expression of the nucleic acid encoding the therapeutic molecule.

In an aspect of the invention, there is provided a method for increasing titre of adeno-associated virus (AAV) vector genomes in a target cell, tissue or organ, the method comprising contacting the target cell, tissue or organ with an AAV particle or a composition thereof described herein under conditions sufficient for transduction of the AAV particle within the target cell, tissue or organ, wherein vector genomes are increased when compared to an AAV particle not having the capsid insertion.

In an aspect of the invention, there is provided a method for increasing the titre of adeno-associated virus (AAV) vector genomes in a subject in need thereof, the method comprising administering an AAV particle or a composition thereof described herein to the subject, wherein vector genomes are increased when compared to a subject having received an AAV particle not having the capsid insertion.

In another aspect of the invention, there is provided use of an AAV particle or a composition thereof in the preparation of a medicament for increasing the titre of adeno-associated virus (AAV) vector genomes in a subject in need thereof, wherein vector genomes are increased when compared to a subject having received an AAV particle not having the capsid insertion.

In another aspect of the invention, there is provided an AAV particle or a composition thereof for use in increasing titre of adeno-associated virus (AAV) vector genomes in a subject in need thereof, wherein vector genomes are increased when compared to a subject having received an AAV particle not having the capsid insertion.

Any embodiment herein shall be taken to apply mutatis mutandis to any other embodiment unless specifically stated otherwise.

The present invention is not to be limited in scope by the specific embodiments described herein, which are intended for the purpose of exemplification only. Functionally equivalent products, compositions and methods are clearly within the scope of the invention, as described herein.

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or group of compositions of matter.

The invention is hereinafter described by way of the following non-limiting Examples and with reference to the accompanying figures.

### BRIEF DESCRIPTION OF THE ACCOMPANYING DRAWINGS

FIG. 1. Generation of modified AAV2/8 vector to improve AAV titre. A. AAV8 cap gene was cloned into the pRC-AAV2 vector in place of the AAV2 cap gene to create the recombinant plasmid pAAV2/8. Endogenous AAV2 p5 promoter was cloned upstream of the AAV2 rep gene and the initiator methionine was mutated from ATG to ACG so that translation would begin at a downstream, in-frame methionine to prevent translation of the two largest Rep isoforms (p78 and p68) which negatively regulate AAV replication. This resulted in a 1.67-fold increase in AAV titre. AAV2 p5 promoter was cloned downstream of the AAV8 cap gene to act as an enhancer element. This resulted in a further 1.74-fold increase in viral titre. B. Y447F, T494V and Y733F amino acid substitutions were introduced into the AAV8 cap ORF by overlapping PCR using KOD Hot Start DNA polymerase. All increased AAV titre, with T494V (1.41-fold) and Y733F (1.33-fold) being most effective. The combination of all three substitutions increased viral titre 1.75-fold.

FIG. 2. Schematic representation of the construction of plasmid libraries encoding AAV2/8 capsid-modified viral particles. A. Pools of randomly generated oligonucleotides encoding peptide sequences were cloned into an AAV2/8 genome-encoding plasmid immediately 3′ to amino acid N590 of capsid protein VP1 using introduced pairs of restriction sites. The cap nucleotide sequence is shown in lowercase, the corresponding amino acid sequence is in uppercase, restriction sites are shown in blue and the amino acid immediately upstream of the site of insertion is shown in red. B. Vector map of recombinant AAV2/8 Y447F, T494V, Y733F triple mutant vector with AAV2 p5 promoters.

FIG. 3. Construction of random peptide display library within loop IV (VR-VIII) of the AAV2/8 capsid. A. AAV2/8 Y447F, T494V, Y733F triple mutant vector p5 promoter and 3′ enhancer sequences were flanked with by AAV2 inverted terminal repeats (ITRs) to display random peptide library within loop IV of the AAV2/8 capsid. N590 library displays random seven-amino acid peptides flanked by two additional fixed amino acids at their N-terminus and three at their C-terminus. DNA sequencing of three clones from N590 library verified that different random peptides were encoded by each clone. B. Sbfl digestion of each clone (and library) confirmed the presence ITRs.

FIG. 4. AAV8 N590 Library selection ex vivo. To identify novel AAV variants, a five round selection method of initial AAV library was performed in human retina and subretinal tissue. Following the selection process, novel AAV variants were recloned into bacterial plasmid not containing ITR-2 elements and sequenced. Selected clones were AAV vector produced using triple transfection in AAV293 cells and used in functional assays.

FIG. 5. Functional assessment of AAV8 variants on immunogenicity. Cos-7, LnCap and Mel cells in 24-well and 96-well plates were transduced with recombinant AAV2/8 and AAV2/8 C1m1 vectors of the same titre using secNanoLuc luciferase as reporter.

FIG. 6. Comparison of peptide inserts into loop IV of the AAV8 capsid. Clustal O (1.2.4) multiple sequence alignment (https://www.ebi.ac.uk/Tools/msa/clustalo/) of peptide inserts into position N590 of capsid protein VP3 AAV8. MView1.63 was used for graphical representation of alignment (Brown et al., 1998). Inserts obtained after AAV8 N590 Library selection ex vivo (FIG. 6) were compared to published sequences of inserts at N590 of wild type AAV2 and AAV8 capsids: AAV-7m8 (Khabou et al, 2016), AAV2.NN, AAV2.GL (Pavlou et al, 2021), AAV8_lung, AAV8_breast (Büning and Srivastava, 2019), AAV8_libNG (Börner et al, 2020).

FIG. 7. Analysis of reporter fluorescence of various AAV-GFP optimised clones. ARPE19 cells were transduced in triplicate with AAVs containing AAV-CMV-GFP reporter plasmid (Addgene #67634) and packaged with 7m8, AAV2/8 p5-2, Clm1 and Clm2 capsids. Shown are representative fluorescence microscopy images of ARPE19 cells transduced with the indicated AAV variants expressing GFP. Both Clm1 and Clm2 AAV2/8 capsids demonstrated GFP expression at 24 hr and 48 hr after transduction showing that they as efficient at transducing ARPE19 cells as “benchmarking” AAV2-7m8 variant, which is currently being tested in human clinical trials via intravitreal injection into the eye (ClinicalTrials.gov: NCT03326336), and substantially more efficient than parental AAV2/8 p5-2 capsid.

FIG. 8. Testing of reporter fluorescence of various AAV-GFP and tdTomato optimised clones in primary retinal cell lines. Human primary RPE mixed cultures (passage 4-7) were transduced in triplicate with AAVs containing tdTomato reporter plasmid (tdTomato cloned in pAAV-MCS, CellBiolabs), AAVs containing AAV-CMV-GFP reporter plasmid (Addgene #67634) and packaged with 7m8, AAV2/8 p5-2, C1m1 and C1m2 capsids. Shown are representative fluorescence microscopy images of human primary RPE cells transduced with the indicated AAV variants expressing GFP or TdTomato. Both C1m1 and C1m2 AAV2/8 capsids demonstrated GFP and tdTomato expression at 48 hr after transduction showing that they as efficient at transducing primary RPE cells as “benchmarking” AAV2-7m8 variant. Parental AAV2/8 p5-2 capsid showed negligible transduction of primary RPE cell cultures.

FIG. 9. Transduction assays with secNanoLuc AAVs C1m1 and C1m2 in ARPE19 and primary RPE cell lines. A-B) Both C1m1 and C1m2 AAV2/8 capsids demonstrated robust expression of secNanoLuc luciferase in ARPE19 and primary RPE cells. Accumulation of luciferase at day 5 and day 7 after transduction showing that they are more efficient at transducing human retinal pigment cells compared to AAV2-7m8 variant and parental AAAV2/8 p5-2 capsid. Relative transduction efficiency was especially high for C1m2 capsid on day 5 (up to 6-fold difference in comparison to AAV2-7m8 variant). These differences were observed during the whole duration of experiment (7 days). At day seven post-transduction, cells transduced using C1m2 AAV2/8 had significantly higher luciferase activity than AAV2-7m8 variant (FIGS. 9A and B). Therefore, peptide-inserts C1m1 and C1m2 at N590 of AAV8 capsid showed high transduction efficiency in human retinal cells.

FIG. 10. Novel AAV8 variants with modified capsid sequences C1m1 and C1m2 efficiently transduce human retinal explants. A-B. Both C1m1 and C1m2 AAV2/8 capsids demonstrated robust expression of secNanoLuc luciferase at day 1 and day2 after transduction showing that they are more efficient at transducing human retinal explants compared to AAV2-7m8 variant and parental AAAV2/8 p5-2 capsid. Relative transduction efficiency was especially high for C1m2 capsid on day 2 (up to 20-fold difference in comparison to AAV2-7m8 variant). These differences were continued during the whole duration of experiment (14 days). At day seven post-transduction, retinal explants transduced using C1m2 AAV2/8 had significantly higher luciferase activity than AAV2-7m8 variant (FIG. 10B). Therefore, peptide-inserts C1m1 and C1m2 at N590 of AAV8 capsid showed high transduction efficiency of human retinal explants. AAV2-7m8 variant was also more efficient at transducing mature photoreceptors, Müller, and ganglion cells, compared to first-generation AAV variants.

FIG. 11. Novel AAV8 variants with modified capsid sequences C1m1 and C1m2 transduced human retinal-pigment epithelium, choroid and sclera (RCS) explants. A-B. Both C1m1 and C1m2 AAV2/8 capsids demonstrated expression of secNanoLuc luciferase in retinal explants at day 1 and day2 after transduction showing that they are as efficient at transducing human RCS explants as “benchmarking” AAV2-7m8 variant and more efficient than parental AAAV2/8 p5-2 capsid.

FIG. 12. Novel AAV8 variants with modified capsid sequences C1m1 and C1m2 efficiently transduce different cell types. A-B. Ocular melanoma cells lines Mel290, 92-1 (A), LX2 Hepatic stellate cells, Huh 7 hepatocellular carcinoma cells (B) and transformed human umbilical vein endothelial cells, EA.hy926 (C) were transduced in triplicate with AAVs containing secNanoLuc reporter and packaged with 7m8, AAV2/8 p5-2, C1m1 and C1m2 capsids. Cells were seeded in 24 well plates, 2×10⁴ cells/well in DMEM/F12 supplemented with 10% v/v foetal bovine serum (FBS) (Gibco) and 100 U/ml penicillin and 100 μg/ml streptomycin (Life Technologies) and transduced with AAVs at MOI 1×10⁴. Cells were transduced with AAVs in DMEM/F12 supplemented with 1% v/v FBS for 24 hrs. Following AAV transduction cells were cultured in 500 μL of DMEM/F12 (phenol red free) medium supplemented with 10% FBS (Gibco) and 100 U/ml penicillin and 100 μg/ml streptomycin (Life Technologies). 50 μL media aliquots were removed at the time points shown and frozen at −80° C. Aliquots were processed for measurement of secNanoLuc activity using Nano-Glo® Luciferase Assay system as recommended by manufacturer (Promega) and CLARIOstar Plus plate reader (BMG Labtech). Data for multiple time points were processed in Excel using transformation to calculate total luminescence at each time point (Promega). Total calculated RLU were analysed by GraphPad Prism 9 software.

FIG. 13. Novel AAV8 variants with modified capsid sequences C1m1 and C1m2 transduce rat retinal epithelium, choroid and sclera (RCS) explants and primary rat RPE cell lines. A. Rat RCS explants were transduced in triplicate with AAVs containing secNanoLuc reporter and packaged with 7m8, AAV2/8 p5-2, C1m1 and C1m2 capsids. Aliquots were processed for measurement of secNanoLuc activity using Nano-Glo® Luciferase Assay system as recommended by manufacturer (Promega) and CLARIOstar Plus plate reader (BMG Labtech). Gain was adjusted to allow direct comparison of RLU between experiments with human and rat RCS explants. Data for multiple time points were processed in Excel using transformation to calculate total luminescence at each time point (Promega). B. Primary rat RPE mixed cell were transduced with AAVs in DMEM/F12 supplemented with 1% v/v FBS for 24 hrs. Aliquots were processed for measurement of secNanoLuc activity using Nano-Glo® Luciferase Assay system as recommended by manufacturer (Promega) and CLARIOstar Plus plate reader (BMG Labtech). Gain was adjusted to allow direct comparison of RLU between experiments with human and rat cell cultures. Data for multiple time points were processed in Excel using transformation to calculate total luminescence at each time point (Promega).

FIG. 14. Transduction assays with potential therapeutic gene IL10 in primary RPE cell lines using modified capsids C1m1 and C1m2. Primary human RPE cells were transduced in triplicate with AAVs containing IL 10 gene and packaged with 7m8, AAV2/8 p5-2, C1m1 and C1m2 capsids. AAV vectors were prepared by triple transfection of Rep/cap plasmids (AAV 7m8, AAV2/8 p5-2, C1m1 and C1m2), IL-10-pAAV and pHelper plasmids into AAV293 cell line (Cell Biolabs). AAV-CMV-TdTomato was used as a non-specific control. Cells were seeded in 24 well plates, 5×10⁴ cells/well in DMEM/F12 supplemented with 10% v/v foetal bovine serum (FBS) (Gibco) and 100 U/ml penicillin and 100 μg/ml streptomycin (Life Technologies) and transduced with AAVs at MOI 1×10⁴. Cells were transduced with AAVs in DMEM/F12 supplemented with 1% v/v FBS for 24 hrs. Following AAV transduction cells were cultured in 500 μL of DMEM/F12 (phenol red free) medium supplemented with 10% FBS (Gibco) and 100 U/ml penicillin and 100 μg/ml streptomycin (Life Technologies). 100 μL media aliquots were removed at the time points shown and frozen at −80° C. Aliquots were processed for measurement of IL10 protein using IL 10 ELISA kit as recommended by manufacturer (Thermo Fisher Scientific). Data for multiple time points were analysed by GraphPad Prism 9 software.

FIG. 15. Novel AAV8 variants with modified capsid sequences C1m1 and C1m2 expressing Endostatin-Angiostatin fusion protein regulate choroidal sprouting in ex vivo human explants. A. Retinal explants were transduced in triplicate with AAVs containing secreted Endostatin-Angiostatin fusion gene and packaged with C1m1 and C1m2 capsids in DMEM/F12 supplemented with 1% v/v FBS for 48 hrs. Endostatin-Angiostatin AAVs were added at 2×10⁹ vg per explant. Secreted Endostatin-Angiostatin fusion gene was cloned into pAAV-MCS (Cell Biolabs). Secreted Endostatin-Angiostatin fusion gene was expressed using the human cytomegalovirus (CMV) promoter. Control explants were left untreated or treated with non-specific AAV containing fluorescent reporter gene. The choroidal sprouts originating from explants were photographed on day 7. Individual explants were photographed using phase contrast optics on Olympus IX73 microscope at 4× magnification. The representative images of choroidal sprouting are shown. B. The sprouting area was quantified using FIJI/Image J (Schindelin et al, 2012). C. The quantification results of sprouting area after transduction with AAV-C1m2 expressing Endostatin-Angiostatin fusion gene are shown (n=3 biological replicates).

### Key to the Sequence Listing

SEQ ID NO: 1—Amino acid sequence, capsid insertion
SEQ ID NOs: 2—108—Amino acid sequence, capsid insertions
SEQ ID NO: 109—Amino acid sequence, capsid insertion
SEQ ID NO: 110—Amino acid sequence, capsid insertion
SEQ ID NO: 111—Amino acid linker sequence
SEQ ID NO: 112—Amino acid linker sequence
SEQ ID NO: 113—Nucleic acid sequence, wild type AAV sequence including AAV2 rep and AAV8 genes
SEQ ID NO: 114-Nucleic acid sequence, wild type AAV sequence including AAV2 rep and AAV8 genes and p5 promoter
SEQ ID NO: 115-Nucleic acid sequence, wild type AAV sequence including AAV2 rep and AAV8 genes, p5 promoter and p5 enhancer
SEQ ID NO: 116-Nucleic acid sequence, AAV sequence including AAV2 rep and AAV8 genes, p5 promoter, p5 enhancer and mutations Y447F Y733F T494V
SEQ ID NO:117-Nucleic acid sequence, AAV sequence including AAV2 rep and AAV8 genes, p5 promoter, p5 enhancer and mutations Y447F Y733F T494V (Parental vector used for construction of library having random inserts at N590)
SEQ ID NO:118-Nucleic acid sequence, p5 promoter
SEQ ID NO:119—Nucleic acid sequence, p5 enhancer
SEQ ID NO:120—Nucleic acid sequence, wild-type AAV8 cap
SEQ ID NO:121-Amino acid sequence, wild-type AAV8 cap
SEQ ID NO:122—Nucleic acid sequence, wild-type AAV2 rep
SEQ ID NO:123-Amino acid sequence, wild-type AAV2 rep
SEQ ID NO:124—Nucleic acid sequence, AAV8 cap with mutations Y447F Y733F T494V
SEQ ID NO: 125—Amino acid sequence, AAV8 cap with mutations Y447F Y733F T494V
SEQ ID NO: 126—Nucleic acid sequence, AAV8 cap with mutations Y447F Y733F T494V (having random inserts at N590)
SEQ ID NO: 127—Amino acid sequence, AAV8 cap with mutations Y447F Y733F T494V (having random inserts at N590)
SEQ ID NO:128—Nucleic acid sequence, C1mut1 insertion with linker sequence
SEQ ID NO:129—Amino acid sequence, C1mut1 insertion with linker sequence
SEQ ID NO:130—Amino acid sequence, VP3 sequence comprising insertion with mutations Y447F Y733F T494V
SEQ ID NO:131—Amino acid sequence, VP3 sequence comprising insertion with linker sequence
SEQ ID NO:132—Amino acid sequence, VP3 sequence comprising C1mut1 insertion
SEQ ID NO:133—Amino acid sequence, VP3 sequence comprising C1mut1 insertion with linker sequence
SEQ ID NO: 134—Nucleic acid sequence, AAV8 cap with mutations Y447F Y733F T494V and C1mut1 insertion with linker sequence
SEQ ID NO: 135—Amino acid sequence, AAV8 cap with mutations Y447F Y733F T494V and C1mut1 insertion with linker sequence
SEQ ID NO: 136—Nucleic acid sequence, AAV sequence with AAV2 rep sequence and AAV8 cap sequence with mutations Y447F Y733F T494V and C1mut1 insertion with linker sequence
SEQ ID NO: 137—Nucleic acid sequence, C1mut2 insertion with linker sequence
SEQ ID NO: 138—Amino acid sequence, C1mut2 insertion with linker sequence
SEQ ID NO: 139—Amino acid sequence, VP3 sequence comprising insertion C1mut2 and with mutations Y447F Y733F T494V
SEQ ID NO: 140—Amino acid sequence, VP3 sequence comprising insertion C1mut2 with linker sequences and with mutations Y447F Y733F T494V
SEQ ID NO: 141—Nucleic acid sequence, AAV8 capsid with insertion C1mut2 with linker sequences mutations Y447F Y733F T494V
SEQ ID NO: 142—Amino acid sequence, AAV8 capsid with insertion C1mut2 with linker sequences mutations Y447F Y733F T494V
SEQ ID NO: 143—Nucleic acid sequence, AAV sequence with AAV2 rep sequence and AAV8 cap sequence with mutations Y447F Y733F T494V and C1mut2 insertion with linker sequence

### DETAILED DESCRIPTION OF THE INVENTION

### General Techniques and Definitions

Unless specifically defined otherwise, all technical and scientific terms used herein shall be taken to have the same meaning as commonly understood by one of ordinary skill in the art (e.g., in cell culture, molecular genetics, cell biology, protein chemistry, and biochemistry).

Unless otherwise indicated, the molecular and statistical techniques utilized in the present disclosure are standard procedures, well known to those skilled in the art. Such techniques are described and explained throughout the literature in sources such as, J. Perbal, 1984, J. Sambrook et al., 1989, T. A. Brown (editor) 1991, D. M. Glover and B. D. Hames (editors) 1995 and 1996, and F. M. Ausubel et al. (editors) 1988, including all updates until present, J. E. Coligan et al. (editors) (including all updates until present).

As used in this specification and the appended claims, terms in the singular and the singular forms “a,” “an” and “the,” for example, optionally include plural referents unless the content clearly dictates otherwise. Thus, for example, reference to “a therapeutic molecule” optionally includes one or more therapeutic molecules.

The term “and/or”, e.g., “X and/or Y” shall be understood to mean either “X and Y” or “X or Y” and shall be taken to provide explicit support for both meanings or for either meaning.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

The term “about” and the use of ranges in general, whether or not qualified by the term about, means that the number comprehended is not limited to the exact number set forth herein, and is intended to refer to ranges substantially within the quoted range while not departing from the scope of the invention. As used herein, “about” will be understood by persons of ordinary skill in the art and will vary to some extent on the context in which it is used. If there are uses of the term which are not clear to persons of ordinary skill in the art given the context in which it is used, “about” will mean up to plus or minus 10%, more preferably 5%, more preferably 1%, of the particular term.

### Capsid Modifications

The present invention is directed to the generation of modified adeno-associated virus (AAV) capsid proteins that provide for improved transduction in a target cell and/or reduced immunogenicity.

The AAV shell is assembled from 60 copies of viral proteins (VP), VP1 (87 kDa), VP2 (73 kDa), and VP3 (61 kDa). The conserved core of each VP subunit consists of an eight-stranded, β-barrel motif and an α-helix (Xie et al., 2002). The outer surface of the capsid is formed by large loops that connect the strands of the β-barrel. For example, the residues from amino acids 581-601 of AAV8 encompass finger-like loops in the VP subunit, and form VR-VIII, consistent with the nomenclature in Nam et al., 2007. The amino acid sequences and structural topology of these loops are reported to facilitate tissue tropism and transduction efficiency (Agbande-McKenna and Kleinschmidt, 2011). Furthermore, these residues contribute to the top of the protrusions that surround the icosahedral 3-fold axes, formed through symmetric interactions between the VPs. Thus, this sequence holds a prominent position on the capsid. This region includes sites shown in some serotypes to be critical for heparin sulfate-binding and cellular uptake.

The AAV capsid consists of three overlapping coding sequences, which vary in length due to alternative start codon usage and interact to form a capsid of icosahedral symmetry. These variable proteins are referred to as VP1, VP2 and VP3, with VP1 being the longest and VP3 being the shortest. An AAV particle consists of all three capsid proteins at a ratio of about 1:1:10 (VP1:VP2:VP3). VP3, which is comprised in VP1 and VP2 at the N-terminus, is the main structural component that builds the particle. The capsid proteins, which are controlled by the same promoter, designated p40, are translated from the same mRNA.

A capsid protein as described herein can be referred to using several different numbering systems. For convenience, as used herein, the AAV sequences are referred to using VP1 numbering, which starts with amino acid 1 for the first residue of VP1 and is then numbered sequentially (i.e., amino acid 2, 3, 4, 5, 6 etc). However, the capsid proteins described herein include VP1, VP2 and VP3 (used interchangeably herein with vp1, vp2 and vp3) with insertions in the corresponding region of the protein. In AAV8, the variable proteins correspond to VP1 (amino acid 1 to 738), VP2 (amino acid 138 to 738), and VP3 (amino acid 203 to 738) using the numbering of the full length VP1. For clarity, when referring to the insertion, it generally refers to an insertion at amino acid 590 of the VP1, at amino acid 590 of the VP2, or at amino acid 590 of the VP3 of AAV8, if using the first amino acid of the particular VP capsid sequence as amino acid 1.

In an embodiment, the insertion is in loop IV, Region 6 or variable region VIII of an AAV8 capsid protein or in a corresponding position of a capsid protein of an AAV serotype other than AAV8. The numbering system, designation of loops, regions and variable regions is consistent with the nomenclature and naming used in Raupp et al., 2012, hereby incorporated in its entirety by reference.

In an embodiment, the capsid of the wild-type AAV8 serotype is used for generating a modified capsid protein of the invention. An example of the amino acid sequence of a wild type AAV8 serotype is shown according to SEQ ID NO: 121.

As used herein, the term “wild type” refers to the native AAV sequence without insertion of the capsid protein of the invention. It may be used interchangeably with the term “parental” and may include sequences that contain other non-naturally occurring modifications. In other words, whilst a naturally occurring AAV may be the source of the wild type sequence to which a modification is made, non-naturally occurring AAV, including, without limitation, recombinant, modified or altered, chimeric, hybrid, synthetic, artificial, etc. AAV may be used as the starting material for which an insertion described herein is introduced. This includes AAV with insertions, mutations or substitutions in regions of the capsid other those the subject of the invention. For example, the AAV may comprise one or more or all of the mutations Y447F Y733F T494V (e.g., a sequence according to SEQ ID NO:125) which may be used as the sequence to which a recombinant capsid protein of the invention is inserted.

Where use of a modified AAV capsid protein or AAV particle is contemplated, it will be understood that the term “modified” refers to AAV capsid proteins or AAV particles that comprise an insertion described herein. The modified AAV capsid protein or AAV particle may however contain other modifications (e.g., mutations) for example, the purpose of improving transduction efficiency.

It will be understood that whilst it is preferable that the AAV8 serotype is used for generating a modified capsid protein, modifications in the homologous region of other AAV serotype capsids are also encompassed by the invention. Other AAV which are useful as wild type sequences include, without limitation, including, e.g. pi.1, pi.2, pi.3, rh.38: rh.40; rh.43; rh.49; rh.50, rh.51; rh.52; rh.53; rh.57; rh.58; rh.61; rh.64; hu.6; hu. 17; hu.37; hu.39; hu.40; hu.41; hu.42; hu. 66; and hu.67. Still other AAV which are useful as wild type sequences include AAV1, AAV2, AAV3, AAV4, AAV5, AAV6, AAV7, AAV9 and shH10AAV. In another embodiment, the AAV wild type sequence is a Clade E AAV. In another embodiment, the AAV wild type sequence is a Clade D AAV. A clade is a group of AAV which are phylogenetically related to one another as determined using a Neighbor-Joining algorithm by a bootstrap value of at least 75% (of at least 1000 replicates) and a Poisson correction distance measurement of no more than 0.05, based on alignment of the AAV vp1 amino acid sequence. The Neighbor-Joining algorithm has been described extensively in the literature. See, e.g., M. Nei and S. Kumar, Molecular Evolution and Phylogenetics (Oxford University Press, New York 2000).

Clade E is characterized by containing the previously described AAV8 (Gao et al., 2002), 43.1/rh.2; 44.2/rh.10: rh. 25; 29.3/bb. 1; and 29.5/bb.2 US Patent Publication No. US 2003/0138772 A1. Additional clade E sequences are described in U.S. Pat. No. 7,096,111, which is incorporated herein by reference. AAV7 belongs to clade D and is closest related to the clade E members AAV8 and AAV10 based on VP1 or VP3 amino acid sequence ranging from 85 to 88% aa sequence identity. When AAV7 is superposed onto AAV8, the overall Ca RMSD is 0.75 Å with a structural identity of 93%, which is slightly lower than the comparison to AAV2 described above at 0.92 Å (Mietzsch et al., 2021).

As the transduction efficiency of AAV particles can be determined by the nature of the amino acid residues exposed at the surface of the capsid (Wu et al., 2006), engineering the amino acids of the capsid proteins is one approach to modify transduction efficiency. However, substitution of particular amino acids of the capsid protein can cause it to misfold. One approach to minimise misfolding is to make modifications to the variable regions only within the capsid. By way of example, the variable regions of the capsid of AAV8 are listed in Table 1 below.

TABLE 1
Variable regions of the AAV8 capsid

| Variable region | Corresponding amino acids |
|---|---|
| VRI | 261-273 |
| VRII | 325-330 |
| VRIII | 381-384 |
| VRIV | 450-466 |
| VRV | 490-503 |
| VRVI | 527-532 |
| VRVII | 545-556 |
| VRVIII | 581-601 |
| VRIX | 704-713 |

Screening of an AAV8 capsid library according to the methods described in the Examples herein has led to the identification of modified AAV8 variant capsid proteins that display increased transduction efficiency and reduced immunogenicity when compared to an AAV particle that does not comprise the modified capsid protein.

In some embodiments, a modified recombinant AAV (e.g., AAV8) capsid protein has one or more amino acid insertions in any one variable region (VR) (e.g., VRI, VRII, VRIII, VRIV, VRV, VRVI, VRVII, VRVIII or VRIX), preferably in VRVIII. In some embodiments, in addition to the insertions described herein, a recombinant rAAV (e.g., variant rAAV8) capsid protein may have one or more amino acid mutations in more than one variable region (e.g., VRI and VRII. VRI and VRVII, VRV and VRVII, VRV and VRI and VRVII or VRIV and VRII). It should be understood that recombinant rAAV (e.g., recombinant rAAV8) capsid proteins disclosed herein can have one or more amino acid mutations in any combination of more than one variable regions and is not limited to the examples herein.

In one example, the recombinant rAAV (e.g., recombinant rAAV8) capsid protein has an insertion in any one of the variable regions of the AAV VP1, VP2 or VP3 proteins, or any combination thereof. It is known in the art that when encoded by a single gene, VP1, VP2 and VP3 share most of their amino acids. Specifically, the entire VP3 sequence is also contained within VP2 and VP1. Thus a skilled person will understand that whilst the capsid modifications have been made to VP3 as described herein, the modifications are also applicable to VP1 and VP2 of the AAV capsid, preferably within VR-VIII of VP3, most preferably at amino acid position 590 of VR-VIII of VP3. Preferably the VP1, VP2 and VP3 capsid proteins belong to the AAV8 serotype.

A skilled person will understand how to determine a corresponding position of a capsid protein of an AAV serotype other than AAV8 based on known methods in the art. For example, a sequence alignment of the AAV amino acids may be carried out to determine differences in the amino acid sequence as per the approach taken in Nam et al., 2007. In one example, a multiple sequence alignment program may be used such as ClustalW.

Whilst the invention preferably contemplates use of a recombinant AAV capsid comprising the modification according to SEQ ID NO:1, it will be understood that amino acid sequences containing conservative variations that either maintain or increase transduction efficiency (or show similar or increased immunogenicity) when compared to the effect of a recombinant AAV capsid comprising the modification according to SEQ ID NO:1 are also encompassed by the current invention. Sequences containing conservative variations when compared to SEQ ID NO:1, SEQ ID NO: 109 or SEQ ID NO:110 that either maintain or increase transduction efficiency (or show similar or increased immunogenicity) are considered to be functional equivalents that fall within the scope of the present invention. Thus, in an example, functional equivalents (i.e., having maintained or increased transduction efficiency) of SEQ ID NO:1, SEQ ID NO:109 or SEQ ID NO: 110 fall within the scope of the present invention.

Details of such conservative amino acid changes are provided in Table 2. As the skilled person would be aware, such minor changes can reasonably be predicted not to alter the activity of the polypeptide when expressed in a recombinant cell. Such sequences have at least one amino acid residue in the polypeptide molecule removed and a different residue inserted in its place. These sites are preferably substituted in a relatively conservative manner in order to maintain the desired activity.

TABLE 2
Exemplary substitutions.

| Original Residue | Exemplary Substitutions |
|---|---|
| Ala (A) | val; leu; ile; gly |
| Arg (R) | lys |
| Asn (N) | gln; his |
| Asp (D) | glu |
| Cys (C) | ser |
| Gln (Q) | asn; his |
| Glu (E) | asp |
| Gly (G) | pro, ala |
| His (H) | asn; gln |
| Ile (I) | leu; val; ala |
| Leu (L) | ile; val; met; ala; phe |
| Lys (K) | arg |
| Met (M) | leu; phe |
| Phe (F) | leu; val; ala |
| Pro (P) | gly |
| Ser (S) | thr |
| Thr (T) | ser |
| Trp (W) | tyr |
| Tyr (Y) | trp; phe |
| Val (V) | ile; leu; met; phe, ala |

Thus, in an embodiment, the modified recombinant AAV (e.g., recombinant rAAV8) capsid protein comprises any one or more of the modifications listed in Table 2 and thus have a sequence according to any one of SEQ ID Nos: 2-108.

TABLE 3
Examples of AAV caspid insertions
Modification

GNRVD (SEQ ID NO: 1)
PNRVD (SEQ ID NO: 2)
ANRVD (SEQ ID NO: 3)
GQRVD (SEQ ID NO: 4)
GHRVD (SEQ ID NO: 5)
PQRVD (SEQ ID NO: 6)
PHRVD (SEQ ID NO: 7)
AQRVD (SEQ ID NO: 8)
AHRVD (SEQ ID NO: 9)
GNKVD (SEQ ID NO: 10)
PNKVD (SEQ ID NO: 11)
ANKVD (SEQ ID NO: 12)
GQKVD (SEQ ID NO: 13)
GHKVD (SEQ ID NO: 14)
PQKVD (SEQ ID NO: 15)
PHKVD (SEQ ID NO: 16)
AQKVD (SEQ ID NO: 17)
AHKVD (SEQ ID NO: 18)
GNRID (SEQ ID NO: 19)
AQRAD (SEQ ID NO: 20)
AHRID (SEQ ID NO: 21)
AHRLD (SEQ ID NO: 22)
AHRMD (SEQ ID NO: 23)
AHRFD (SEQ ID NO: 24)
AHRAD (SEQ ID NO: 25)
GNKID (SEQ ID NO: 26)
GNKLD (SEQ ID NO: 27)
GNKMD (SEQ ID NO: 28)
GNKFD (SEQ ID NO: 29)
GNKAD (SEQ ID NO: 30)
PNKID (SEQ ID NO: 31)
PNKLD (SEQ ID NO: 32)
PNKMD (SEQ ID NO: 33)
PNKFD (SEQ ID NO: 34)
PNKAD (SEQ ID NO: 35)
ANKID (SEQ ID NO: 36)
ANKLD (SEQ ID NO: 37)
ANKMD (SEQ ID NO: 38)
GNRLD (SEQ ID NO: 39)
GNRMD (SEQ ID NO: 40)
GNRFD (SEQ ID NO: 41)
GNRAD (SEQ ID NO: 42)
PNRID (SEQ ID NO: 43)
PNRLD (SEQ ID NO: 44)
PNRMD (SEQ ID NO: 45)
PNRFD (SEQ ID NO: 46)
PNRAD (SEQ ID NO: 47)
ANRID (SEQ ID NO: 48)
ANRLD (SEQ ID NO: 49)
ANRMD (SEQ ID NO: 50)
ANRFD (SEQ ID NO: 51)
ANRAD (SEQ ID NO: 52)
GQRID (SEQ ID NO: 53)
GQRLD (SEQ ID NO: 54)
GQRMD (SEQ ID NO: 55)
GQRFD (SEQ ID NO: 56)
GQRAD (SEQ ID NO: 57)
ANKFD (SEQ ID NO: 58)
ANKAD (SEQ ID NO: 59)
GQKID (SEQ ID NO: 60)
GQKLD (SEQ ID NO: 61)
GQKMD (SEQ ID NO: 62)
GQKFD (SEQ ID NO: 63)
GQKAD (SEQ ID NO: 64)
GHKID (SEQ ID NO: 65)
GHKLD (SEQ ID NO: 66)
GHKMD (SEQ ID NO: 67)
GHKFD (SEQ ID NO: 68)
GHKAD (SEQ ID NO: 69)
PQKID (SEQ ID NO: 70)
PQKLD (SEQ ID NO: 71)
PQKMD (SEQ ID NO: 72)
PQKFD (SEQ ID NO: 73)
PQKAD (SEQ ID NO: 74)
PHKID (SEQ ID NO: 75)
PHKLD (SEQ ID NO: 76)
GHRID (SEQ ID NO: 77)
GHRLD (SEQ ID NO: 78)
GHRMD (SEQ ID NO: 79)
GHRFD (SEQ ID NO: 80)
GHRAD (SEQ ID NO: 81)
PQRID (SEQ ID NO: 82)
PQRLD (SEQ ID NO: 83)
PQRMD (SEQ ID NO: 84)
PQRFD (SEQ ID NO: 85)
PQRAD (SEQ ID NO: 86)
PHRID (SEQ ID NO: 87)
PHRLD (SEQ ID NO: 88)
PHRMD (SEQ ID NO: 89)
PHRFD (SEQ ID NO: 90)
PHRAD (SEQ ID NO: 91)
AQRID (SEQ ID NO: 92)
AQRLD (SEQ ID NO: 93)
AQRMD (SEQ ID NO: 94)
AQRFD (SEQ ID NO: 95)
PHKMD (SEQ ID NO: 96)
PHKFD (SEQ ID NO: 97)
PHKAD (SEQ ID NO: 98)
AQKID (SEQ ID NO: 99)
AQKLD (SEQ ID NO: 100)
AQKMD (SEQ ID NO: 101)
AQKFD (SEQ ID NO: 102)
AQKAD (SEQ ID NO: 103)
AHKID (SEQ ID NO: 104)
AHKLD (SEQ ID NO: 105)
AHKMD (SEQ ID NO: 106)
AHKFD (SEQ ID NO: 107)
AHKAD (SEQ ID NO: 108)

A skilled person will understand that similar modifications can be made to the sequences GNRVDAH (SEQ ID NO: 109) and GNRVDDF (SEQ ID NO: 110) by making conservative amino acid changes such as those outlined in Table 2.

The term “peptide” is used interchangeably with the term “polypeptide.” A polypeptide or class of polypeptides may be defined by the extent of identity (% identity) of its amino acid sequence to a reference amino acid sequence, or by having a greater % identity to one reference amino acid sequence than to another. The % identity of a polypeptide to a reference amino acid sequence is typically determined by GAP analysis (Needleman and Wunsch, 1970; GCG program) with parameters of a gap creation penalty=5, and a gap extension penalty-0.3. In an embodiment, the query sequence is at least 5 amino acids in length and the GAP analysis aligns the two sequences over a region of at least 5 amino acids. In another embodiment, the query sequence is at least 6 amino acids in length and the GAP analysis aligns the two sequences over a region of at least 6 amino acids. In another embodiment, the query sequence is at least 7 amino acids in length and the GAP analysis aligns the two sequences over a region of at least 7 amino acids. Even more preferably, the GAP analysis aligns two sequences over the entire length of the reference amino acid sequence. The polypeptide or class of polypeptides may have the same activity as, or a different activity than, or a better activity of, the reference polypeptide (e.g., SEQ ID NO:1). In an embodiment, the modified recombinant AAV (e.g., recombinant rAAV8) capsid protein may comprise an amino acid sequence having a degree of homology to any one or more of the modifications listed in Table 3.

For example, the recombinant AAV (e.g., recombinant rAAV8) capsid protein may comprise an amino acid sequence having at least 35%, more preferably at least 40%, more preferably at least 45%, more preferably at least 50%, more preferably at least 55%, more preferably at least 60%, more preferably at least 65%, more preferably at least 70%, more preferably at least 75%, more preferably at least 76%, more preferably at least 80%, more preferably at least 85%, more preferably at least 90%, more preferably at least 91%, more preferably at least 92%, more preferably at least 93%, more preferably at least 94%, more preferably at least 95%, more preferably at least 96%, more preferably at least 97%, more preferably at least 98%, more preferably at least 99%, more preferably at least 99.1%, more preferably at least 99.2%, more preferably at least 99.3%, more preferably at least 99.4%, more preferably at least 99.5%, more preferably at least 99.6%, more preferably at least 99.7%, more preferably at least 99.8%, and even more preferably at least 99.9% identical to the relevant nominated SEQ ID NO, preferably SEQ ID NO:1. Where the modified recombinant AAV (e.g., recombinant rAAV8) capsid protein may comprises an amino acid sequence having 100% identity to the nominated SEQ ID NO, the amino acid sequence is identical to the nominated SEQ ID NO.

In another example, the recombinant AAV (e.g., recombinant rAAV8) capsid protein may comprise one, two or three amino acids that are different to the sequence defined according to SEQ ID NO: 1. Examples of suitable peptides are outlined in Table 3.

A skilled person will understand that a capsid insert may contain a spacer. An example of a spacer suitable for use in the invention is a linker. The term “linker” or “linker region” refers to an oligo- or polypeptide region of from about 1 to 30 amino acids that links together any of the above capsid insertions within the capsid gene, optionally at a position corresponding to amino acid 590. Optionally, a short oligo- or polypeptide linker, e.g., between 2 and 10 amino acids in length, may form the linkage between the capsid insert and the sequence of the capsid gene. A glycine-serine doublet is an example of a suitable linker. For example, in one aspect, the linker includes the amino acid sequence of TG and/or GLS according to SEQ ID NOs: 111 and 112 respectively. In another example, the linker includes the amino acid sequence of GGGS, GGGSGGGS or GGGGSGGGGS. Preferably the linker comprises a short sequence formed by amino acids G and A. A skilled person will understand that when determining a suitable linker for use in the invention described herein, linkers are often composed of G, A, S amino acids and the sequence can be determined based on their properties and interactions with AAV flexible loops.

### Transduction Efficiency and Immunogenicity

In an embodiment of the invention, there are provided methods for increasing the transduction efficiency of an AAV particle in a target cell. Transduction efficiency is understood to include the ability of an AAV particle of the invention, having a modified capsid, to result in increased expression of a gene that is contained within an expression cassette of the AAV particle which may include for instance, a therapeutic molecule or a reporter gene. Alternatively, increased transduction efficiency may be determined by measuring for example, the expression level of the AAV particle itself (i.e., by determining expression of a VP1, 2 or 3 protein by Western blot) or by measuring vector genome quantity. Vector genomes can be measured using known methods in the art or described in the Examples herein. For example, DNA can be isolated using a Qiagen DNA kit, from a target tissue (e.g., the eye) at a study end point and vector copies (vector genomes) quantifiable by ddPCR.

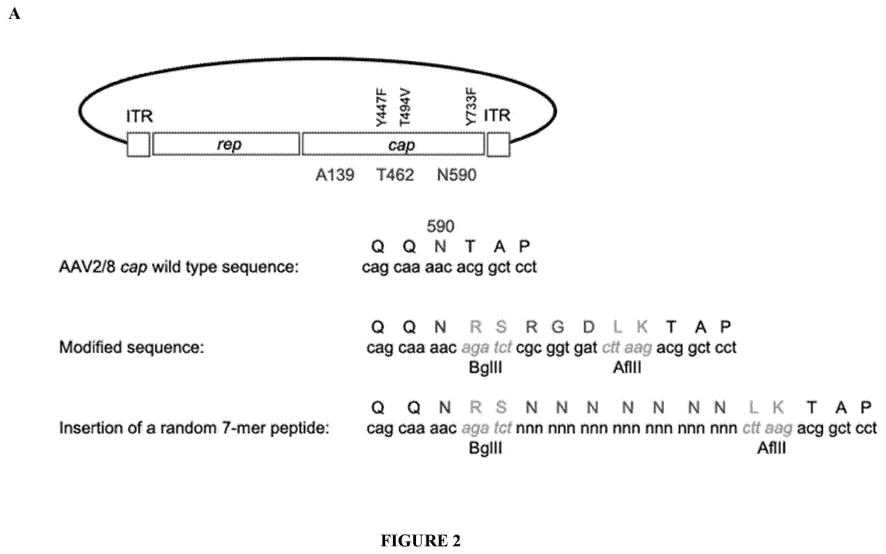

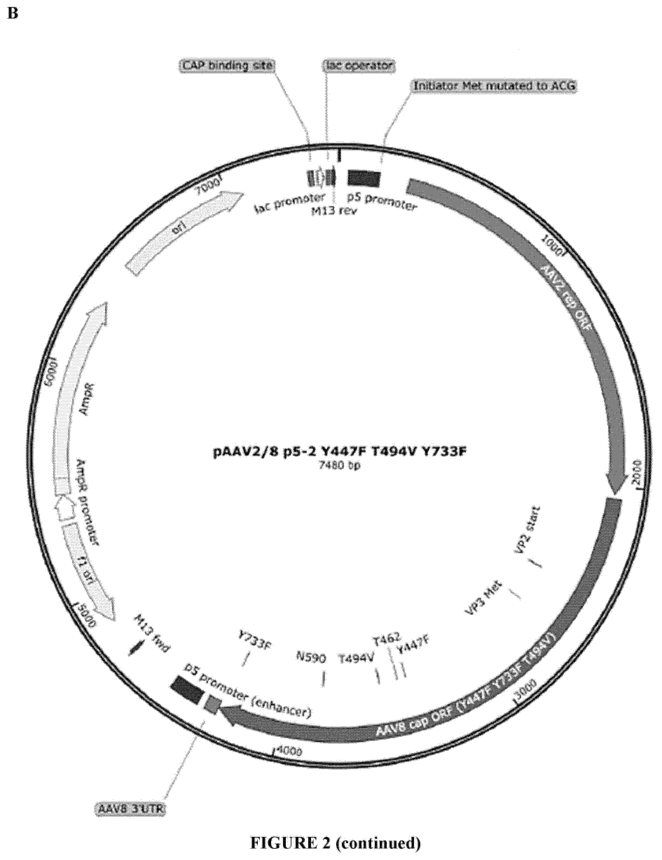

Increased transduction efficiency is intended to be assessed by comparing the AAV particle having a modified capsid to a parental AAV particle that does not comprise the modified capsid in the same cell, tissue or organ type. For example, where the use of an AAV particle comprising AAV8 modified capsid genes described in FIG. 2B is contemplated, the relative comparison will be to an identical AAV particle that does not comprise the modified capsid. The assessment of increased transduction efficiency may also be known as increased infectivity of the target cell and can be determined by methods known in the art including the methods outlined in the Examples herein. In one example, the measurement of vector genomes may be assessed. In this case, the confirmation of an increased amount of vector genomes in a target tissue, cell or organ, when compared to the effect of an AAV particle without the capsid modification, is indicative of increased transduction efficiency. In another method, FACS may be utilised if an AAV particle comprises a reporter such as GFP. In this case, the confirmation of an increased amount of GFP signal from a target tissue, cell or organ, when compared to the effect of an AAV particle without the capsid modification, is indicative of increased transduction efficiency. Yet additional examples may include image cytometry or fluorescence microscopy with image analysis.

Any one of the AAV particles, or compositions comprising a rAAV particle disclosed herein can be used to transduce a target cell, tissue or organ. In some embodiments, a cell, tissue or organ that is transduced using an AAV particle disclosed herein is transduced with a gene of interest comprised within an expression cassette. In some instances, the gene will be a therapeutic molecule with utility in the treatment of an ocular disorder. In some embodiments, a cell, tissue or organ may be transduced in an in vitro setting wherein the cell, tissue or organ is incubated or perfused with a media. A cell may be one of many cells cultured under certain conditions, or part of an organ that is harvested, part of an organoid, or an organism.

In some embodiments, a cell, tissue or organ is transduced in vivo, for example, for the purposes of treating a disease. In some embodiments, such a rAAV particle comprises a gene of interest (i.e., therapeutic molecule) that encodes a therapeutic protein or RNA. In some embodiments there is provided a composition for transducing a cell or tissue of an eye (or two eyes) or brain. In some embodiments, a specific tissue in the eye (or two eyes) or brain is targeted. For example, the retina or one or more cell types of the retina may be targeted. In this case, the target cells may include photoreceptors, retinal ganglion cells, bipolar cells, trabecular meshwork, retinal pigment epithelium cells, amacrine cells, astrocytes, horizontal cells, microglia or Muller glia.

As used herein, the term “ocular cells” refers to any cell in, or associated with the function of the eye. The term may refer to any one or more of photoreceptor cells, including rod, cone and photosensitive ganglion cells, retinal pigment epithelium (RPE) cells, Mueller cells, bipolar cells, horizontal cells, amacrine cells. In one embodiment, the ocular cells are bipolar cells. In another embodiment, the ocular cells are horizontal cells. In another embodiment, the ocular cells are ganglion cells.

In another example of the invention, the AAV particles or compositions thereof have utility in reducing an immune response (i.e., reducing immunogenicity) that may occur when the AAV particle or composition is administered to a target cell, tissue or organ. In some instances, if an AAV particle has high homology with the parental wild-type virus, which has been shown to infect a high percentage of the human population, the administration of an AAV particle can induce an immune response in the subject. This typically presents a limitation to the transduction efficiency of the AAV particle. Both humoral and cell-mediated immunity to wild-type AAV have been documented in healthy donors, and, at least in the case of anti-AAV antibodies, have been shown to have a potentially high impact on the outcome of gene transfer. While several factors can contribute to the overall immunogenicity of rAAV vectors, vector design and the total vector dose appear to be responsible of immune-mediated toxicities. By using an AAV particle with a modified capsid described herein, it is envisaged that a reduced immune response will be observed when administered to a target cell, tissue or organ, and this may increase transduction efficiency.

A reduced immune response or reduced immunogenicity may be assessed by comparing the AAV particle having a modified capsid to a parental AAV particle that does not comprise the modified capsid in the same cell, tissue or organ type. For example, where the use of an AAV particle comprising AAV8 modified capsid genes described in FIG. 2B is contemplated, the relative comparison will be to an identical AAV particle that does not comprise the modified capsid. The assessment of reduced immunogenicity can be determined by methods known in the art including the methods outlined in the Examples herein. In one example, a serum virus neutralisation (SVN) assay may be used. In another example, an assessment of the functional capacity of the immune response can be made by measuring specific cell functions ex vivo (i.e., of the cells isolated and studied in short- or long-term culture).

### AAV Particles

The term “AAV” includes AAV type 1 (AAV1), AAV type 2 (AAV2), AAV type 3 (AAV3), AAV type 4 (AAV4), AAV type 5 (AAV5), AAV type 6 (AAV6), AAV type 7 (AAV7), AAV type 8 (AAV8), AAV type 9 (AAV9), Rh74, AAV 9_hu14, AAVshH10, avian AAV, bovine AAV, canine AAV, equine AAV, primate AAV, non-primate AAV, and ovine AAV as well as other known AAV serotypes in the art.

AAV, a member of the Parvovirus family, is a small nonenveloped, icosahedral virus with single-stranded linear DNA genomes of 4.7 kilobases (kb). AAV is assigned to the genus, Dependovirus, because the virus was discovered as a contaminant in purified adenovirus stocks (D. M. Knipe, P. M. Howley, Field's Virology, Lippincott Williams & Wilkins, Philadelphia, ed. Sixth, 2013). In its wild-type state, AAV depends on a helper virus-typically adenovirus—to provide necessary protein factors for replication, as AAV is naturally replication-defective. The 4.7-kb genome of AAV is flanked by two inverted terminal repeats (ITRs) that fold into a hairpin shape important for replication.

In an aspect, the present invention contemplates the use of recombinant AAV particles which typically contain a modified capsid and expression cassette for expressing a therapeutic molecule of interest, preferably for the treatment of an ocular disorder in a subject in need thereof. The term “recombinant AAV particle” refers to a molecule which has been constructed or modified by recombinant DNA/RNA technology. For example, in an embodiment, the capsid of the AAV particle has been modified and can be considered recombinant itself, whilst forming part of a larger AAV recombinant particle.

The recombinant AAV capsids of the invention will typically be contained within an AAV particle for delivery to a target cell, tissue or organ of interest. An “AAV virion” or “AAV particle” or “AAV vector” refers to a viral particle composed of at least one AAV capsid polypeptide and typically includes an encapsidated polynucleotide expression cassette. If the particle comprises a heterologous nucleic acid (i.e. a polynucleotide other than a wild-type AAV genome, such as a transgene to be delivered to a mammalian cell), it can be referred to as an “AAV vector” or an “AAV expression vector”. The term “AAV particle” may include particles that do not contain a nucleic acid expression cassette comprising a gene of interest but typically relates to particles that contain a nucleic acid expression cassette comprising a gene of interest. In some embodiments, the AAV particle comprises ITRs and/or a rep ORF of an AAV2 serotype. In other embodiments, the AAV particle may be pseudotyped which comprises a modified capsid protein from an AAV8 serotype as well as ITRs and/or a rep ORF of an AAV2 serotype. An example of a suitable pseudotyped AAV particle is that according to the sequence SED ID NO: 136 or 143.

Where it is contemplated that the AAV particles described herein comprise an “expression cassette” these terms refer to a nucleic acid sequence encoding for various nucleic acid sequences, including those that when expressed, target expression to retinal cells of the eye. The expression cassettes can also comprise a heterologous nucleic acid sequence not of AAV origin as part of the nucleic acid insert. This heterologous nucleic acid sequence typically comprises a sequence of interest for the genetic transformation of a cell. In general, the heterologous nucleic acid sequence is flanked by at least one, and generally by two AAV inverted terminal repeat sequences (ITRs). In some cases the ITRs may be of a different serotype to the native AAV. In these cases, the AAV vector may be considered to be a pseudotyped vector.

Being naturally replication-defective and capable of transducing nearly every cell type in the human body, AAV represents an ideal vector for therapeutic use in gene therapy or vaccine delivery. In it's wild-type state, AAV's life cycle includes a latent phase during which AAV genomes, after infection, are site-specifically integrated into host chromosomes and an infectious phase during which, following either adenovirus or herpes simplex virus infection, the integrated genomes are subsequently rescued, replicated, and packaged into infectious viruses. When vectorized, the viral Rep and Cap genes of AAV are removed and provided in trans during virus production, making the ITRs the only viral DNA that remains (Vasileva & Jessberger, Nature reviews. Microbiology (2005)). Rep and Cap are then replaced with an array of possible transfer vector configurations to perform gene addition or gene targeting.

Such vectorized recombinant AAVs (rAAV) transduce both dividing and non-dividing cells, and show robust stable expression in quiescent tissues. The number of rAAV gene therapy clinical trials that have been completed or are ongoing to treat various inherited or acquired diseases is increasing dramatically. In particular, the properties of non-pathogenicity, broad host range of infectivity, including non-dividing cells, and potential site-specific chromosomal integration make AAV an attractive tool for gene transfer. The first rAAV-based gene therapy to be approved in the Western world (Glybera® for lipoprotein lipase deficiency, approved for use in 2012 in the European Union) has stimulated the gene therapy community, investors and regulators to the real possibility of moving rAAV therapies into the clinic globally.

A skilled person may engineer the AAV particle such that it has a suitability for transducing the target cell, tissue or organ. For example, if the subject has a disease, disorder or condition associated with the liver, a skilled person may utilise an AAV serotype that is effective at transducing the liver (e.g., AAV7, AAV8, AAV9). Similarly, if the subject has a disease, disorder or condition associated with the striated muscle of the heart, a skilled person may utilise an AAV serotype that is effective at transducing the heart (e.g., AAV1, AAV8, AAV9). Examples of suitable serotypes, depending on the cell, organ or tissue to be transduced are outlined in Table 4 below.

In an example, the present invention contemplates the expression of a therapeutic molecule within an AAV particle for delivery to the cells of the retina. There are a number of approaches that an AAV particle can be targeted to the cells of the retina. An example may be to use a particle of AAV1, AAV2, AAV4, AAV5 or AAV8 serotype of any pseudotype thereof.

TABLE 4
Examples of suitable AAV serotypes that may be utilised
to target expression to a specific cell, tissue or organ.

| Tissue | Serotype |
|---|---|
| Central Nervous | AAV1, AAV2, AAV4, AAV5, AAV8, AAV9 |
| System | |
| Heart | AAV1, AAV8, AAV9 |
| Kidney | AAV2 |
| Liver | AAV7, AAV8, AAV9 |
| Lung | AAV4, AAV5, AAV6, AAV9 |
| Pancreas | AAV8 |
| Photoreceptor cells | AAV2, AAV5, AAV8 |
| Retinal cells | AAV1, AAV2, AAV4, AAV5, AAV8, shH10AAV |
| Skeletal muscle | AAV1, AAV2, AAV4, AAV5, AAV8, AAV9, rh74 |

Additionally, the AAV particle may contain additional regulatory elements that restrict expression in the target cell, tissue or organ. In the case of the liver, a liver specific promoter may be used. Similarly, for the treatment of a heart condition, disease or disorder, a heart specific promoter may be used. Where the transduction of cells of the retina is contemplated, expression may be restricted by utilising retinal cell specific promoters including those listed in Table 5 below.

TABLE 5
Suitable promoters for restricting expression to cells of the retina

| | Size (bp) | Origin, Cell Expression, Strength |
|---|---|---|
| Müller Glial Cells | | |
| CHX10 | 164 | Retinal progenitor cells |
| GFAP | 2600 | Müller glial cells |
| GFAP | 2600 | Müller glial cells (Novartis) |
| GfaABC1D | 686 | Müller glial cells |
| | | Hypoxia-induced reactive MGC promoter |
| HRSE-6xHRE-GfaABC1D | −820 | HRE is (A/G)CGT(G/C)C. HRSE from |
| | | metallothionein II promoter (90 bps) |
| RLBP1 | 2789 | Müller glial cells |
| Short RLBP1 | 581 | Müller glial cells |
| Murine CD44 | 1775 | Müller glial cells |
| Murine sbCD44 | 363 | Müller glial cells |
| ProB2 | 592 | Müller glial cells |
| Photoreceptor Cells | | |
| Mouse RHO | 1400 | Rod PRCs |
| Human RHO (rhodospin) | 800 | Rod PRCs |
| Human RHO | 520 | Rod- PRCs |
| Mouse rod ospin mOp500 | 500 | Rod- PRCs −385/+86 |
| Mouse rod opsin | 221 | Rod- PRCs |
| | | Rod and cone PRCs. AY327580.1: bp |
| Human Rhodospin kinase | 294 | 1,793-2,087 (−112 to +180). More efficient |
| (RHOK/GRK1) | | than IRBP in NHP for cone transduction |
| Human blue opsin HB570 | 570 | S-cone and subset of M-cones PRCs |
| Human blue opsin HB569 | 569 | blue cone opsin PRCs |
| PR0.5 | 496 | Red cone PRCs |
| PR1.7 | 1700 | Red cone PRCs |
| PR2.1 | 2,100 | Red cone PRCs |
| 3LCR-PR0.5 | −600 | Red cone PRCs |
| Mouse blue opsin (mBP500) | 500 | Mouse s opsin |
| Human interphotoreceptor retinoid | 235 | Cone and rod PRCs |
| binding protein (hIRBP) | | X53044.1, bp 2,603-2,837 |
| IRBPe/GNAT2 | 500 | Cone PRCs |
| Mouse CAR/ARR3 | 500 | Cone PRCs, some rods, and RPE |
| Human CAR/ARR3 | 405-500 | Cone PRCs, some rods, and RPE cells |
| CAR/ARR3 | 215 | Cone PRC |
| Human red opsin | 2,100 | Human red cone opsin |

As used herein, the term ‘promoter’ refers to nucleic acid sequences that regulate, either directly or indirectly, the transcription of corresponding nucleic acid coding sequences to which they are operably linked (e.g. a gene of interest or therapeutic molecule). “Operably linked” refers to the ability to the ability of the promoter or regulatory element to have a functional effect on the transgene to which it is linked.

A promoter may function alone to regulate transcription or may act in concert with one or more other regulatory sequences (e.g. enhancers or silencers, or regulatory elements). In the context of the present invention, a promoter is typically operably linked to a gene of interest or therapeutic molecule to regulate its transcription. When a promoter is operably linked to a gene of interest or therapeutic molecule, the promoter can (1) confer a significant degree of cell, tissue or organ specific expression in vivo or in vitro (e.g., in retinal cells) of the gene, and/or (2) can increase the level of expression of the gene in the target cell, tissue or organ.

The promoter may be homologous (i.e. from the same species as the animal, in particular mammal, to be transfected with the nucleic acid expression cassette) or heterologous (i.e. from a source other than the species of the animal, in particular mammal, to be transfected with the expression cassette). As such, the source of the promoter may be any virus, any unicellular prokaryotic or eukaryotic organism, any vertebrate or invertebrate organism, or may even be a synthetic promoter (i.e. having a non-naturally occurring sequence), provided that the promoter is functional in combination with the regulatory elements described herein. In an embodiment, the promoter is a mammalian promoter, in particular a murine or human promoter. Alternatively, the promoter may be an inducible or constitutive promoter. In an embodiment, the promoter is a chimeric promoter (i.e., containing elements from different species).

To minimize the length of the nucleic acid expression cassette, the regulatory elements may be linked to minimal promoters, or shortened versions of the promoters described herein. A ‘minimal promoter’ (also referred to as basal promoter or core promoter) as used herein is part of a full-size promoter still capable of driving expression, but lacking at least part of the sequence that contributes to regulating (e.g. tissue-specific) expression. This definition covers both promoters from which (tissue-specific) regulatory elements have been deleted—that are capable of driving expression of a gene but have lost their ability to express that gene in a tissue-specific fashion and promoters from which (tissue-specific) regulatory elements have been deleted that are capable of driving (possibly decreased) expression of a gene but have not necessarily lost their ability to express that gene in a tissue-specific fashion. Preferably, the promoter contained in the nucleic acid expression cassette disclosed herein is 1000 nucleotides or less in length, 900 nucleotides or less, 800 nucleotides or less, 700 nucleotides or less, 600 nucleotides or less, 500 nucleotides or less, 400 nucleotides or less, 300 nucleotides or less, or 250 nucleotides or less.

An “inducible” promoter may be a promoter which may be under environmental control and can be used in some embodiments of the invention. Examples of environmental conditions that may initiate transcription by inducible promoters include anaerobic conditions and the presence of light. With an inducible promoter, the rate of transcription increases in response to an inducing agent. Exemplary inducible promoters include, but are not limited to: tetracycline-inducible expression system (or a Tet Response Element (TRE) and tetO) and the inducible promoter from a steroid hormone gene, the transcriptional activity of which may be induced by a glucocorticosteroid hormone.

According to particular embodiments, at least 50%, at least 60%, at least 70%, at least 75%, at least 80%, at least 85%, at least 90%, at least 95%, at least 98%, at least 99% or 100% of the expression of a therapeutic molecule occurs within the target cells, tissue or organ, preferably in cells of the retina. Thus, according to particular embodiments less than 25%, less than 10%, less than 5%, less than 2% or even less than 1% of the expression of a therapeutic molecule occurs in an organ or tissue other than the target cells, tissue or organ, preferably other than cells of the retina.

An approach for targeting expression to a target cell, tissue or organ is to use a nucleic acid regulatory element for increasing gene expression in the cell, tissue or organ. A “regulatory element” as used herein refers to a transcriptional control element, in particular a non-coding cis-acting transcriptional control element, capable of regulating and/or controlling transcription of a gene. A retinal specific regulatory element as used herein refers to a transcriptional control element, in particular a non-coding cis-acting transcriptional control element, capable of regulating and/or controlling transcription of a gene within the retina.

Regulatory sequences, or control elements, refer to nucleotide sequences that influence the timing and level/amount of transcription, RNA processing or stability, or translation of the associated coding sequence. Regulatory sequences may include promoters; translation leader sequences; introns; enhancers; stem-loop structures; repressor binding sequences; termination sequences; polyadenylation recognition sequences; etc. Particular regulatory sequences may be located upstream and/or downstream of a coding sequence operably linked thereto. Regulatory elements may comprise at least one transcription factor binding site (TFBS), more in particular at least one binding site for a tissue-specific transcription factor.

Typically, regulatory elements as used herein increase or enhance gene expression when compared to the transcription of the gene without the regulatory elements. Thus, regulatory elements particularly comprise enhancer sequences, although it is to be understood that the regulatory elements increasing transcription are not limited to typical far upstream enhancer sequences, but may occur at any distance of the gene they regulate. Indeed, it is known in the art that sequences regulating transcription may be situated either upstream (e.g. in the promoter region) or downstream (e.g. in the 3′UTR) of the gene they regulate in vivo, and may be located in the immediate vicinity of the gene or further away. Of note, although regulatory elements as disclosed herein typically comprise naturally occurring sequences, combinations of (parts of) such regulatory elements or several copies of a regulatory element, i.e. regulatory elements comprising non-naturally occurring sequences, are themselves also envisaged as regulatory element. Regulatory elements as used herein may comprise part of a larger sequence involved in transcriptional control, e.g. part of a promoter sequence. However, regulatory elements alone are typically not sufficient to initiate transcription but require a promoter to this end (which may itself be a regulatory element). The regulatory elements disclosed herein are provided as nucleic acid molecules, i.e. isolated nucleic acids, or isolated nucleic acid molecules or polynucleotides.

In the context of the present invention, nucleic acid regulatory elements typically comprise an artificial sequence that include the regulatory elements and are obtained by rearranging the transcription factor binding sites (TFBS) that are present in a recombinant AAV vector. Nucleic acid regulatory elements for increasing gene expression in the retina are known in the art and may be utilised in the present invention.

The present invention also contemplates use of the regulatable expression cassettes for systematically controlling and silencing expression of a therapeutic molecule that are described in PCT/AU2019/050046, the entire contents of which is hereby incorporated by reference. In an example, the invention contemplates the use of an expression cassette comprising a kill switch and a regulatable element operably linked to a nucleic acid sequence encoding a therapeutic molecule, wherein activity of the regulatable element is regulated by a regulator compound, and wherein activation of the kill switch silences expression of the nucleic acid encoding the therapeutic molecule from the cassette. The expression cassette is preferably contained within a viral particle of the invention which comprises a modified AAV capsid protein such as that defined in SEQ ID NO: 1 or functional equivalents thereof.

The regulatable expression cassettes encompassed by the present disclosure comprise a “kill switch”. The term “kill switch” refers to element(s) of cassettes defined herein that can silence expression of a nucleic acid encoding a therapeutic molecule(s) from the cassette. The term “silence” is used in this context to refer to complete and irreversible suppression of expression. In an example, silencing completely abolishes expression of a nucleic acid encoding a therapeutic molecule from an expression cassette defined herein.

In an example, the kill switch can facilitate removal of a nucleic acid sequence encoding a therapeutic molecule(s) or part thereof from the cassette. In another example, the kill switch facilitates removal of some or all of the transcriptional machinery required for expression of the therapeutic molecule(s) from the cassette. In another example, the kill switch facilitates removal of one or more promoters. In another example, the kill switch facilitates removal of a transactivator gene. In another example, the kill switch facilitates removal of the entire expression cassette from the viral vector or AAV particle (i.e. all foreign genes and regulatory elements are removed). In another example, the kill switch facilitates inversion of the sequence encoding one or more of the above elements. For example, the kill switch can facilitate inversion of some or all of the sequence(s) encoding a promoter(s), therapeutic molecule(s) or transactivator.

In the context of the present invention, any intron can be utilized in the expression cassettes described herein. The term “intron” encompasses any portion of a whole intron that is large enough to be recognized and spliced by the nuclear splicing apparatus. Typically, short, functional, intron sequences are preferred in order to keep the size of the expression cassette as small as possible which facilitates the construction and manipulation of the expression cassette. In some embodiments, the intron is obtained from a gene that encodes the protein that is encoded by the coding sequence within the expression cassette. The intron can be located 5′ to the coding sequence, 3′ to the coding sequence, or within the coding sequence. An advantage of locating the intron 5′ to the coding sequence is to minimize the chance of the intron interfering with the function of the polyadenylation signal. In embodiments, the nucleic acid expression cassette disclosed herein further comprises an intron. Non-limiting examples of suitable introns are Minute Virus of Mice (MVM) intron, beta-globin intron (betaIVS-1), factor IX (FIX) intron A, Simian virus 40 (SV40) small-t intron, and beta-actin intron.

Any polyadenylation signal that directs the synthesis of a polyA tail is useful in the expression cassettes described herein, examples of those are well known to one of skill in the art. Exemplary polyadenylation signals include, but are not limited to, polyA sequences derived from the Simian virus 40 (SV40) late gene, the bovine growth hormone (BGH) polyadenylation signal, the minimal rabbit beta-globin (mRBG) gene, and the synthetic polyA s (SPA) site as described in Levitt et al., 1989.

AAV vectors typically comprise inverted terminal repeats (ITRs) of about 145 nt at either end, which contain sequences necessary for DNA replication and packaging into virions for nucleic acid delivery. In one example, the ITRs of AAV serotype 2 are used. However, ITRs from other suitable serotypes may be selected. These ITRs or other AAV components may be readily isolated using techniques available to those of skill in the art from an AAV serotype (see WO 2006/110689).

Various recombinant AAV vector systems have been developed for nucleic acid delivery because they are non-pathogenic and exhibit a broad range of tissue specificity. AAV vectors can be readily constructed using techniques known in the art. See, e.g., U.S. Pat. Nos. 5,173,414 and 5,139,941; International Publication Nos. WO 1992/01070 and WO 1993/03769; Muzyczka., 1992; Kotin, 1994; and Zhou et al., 1994.

In another example, the AAV is a self-complementary AAV (sc-AAV) (see for example, US2012/0141422). Self-complementary AAV vectors package an inverted repeat genome that can fold into dsDNA without the requirement for DNA synthesis or base-pairing between multiple vector genomes.

In an embodiment, the recombinant AAV particle has a sequence that is at least 50%, at least 55%, at least 60%, at least 65%, at least 70%, at least 75%, at least 80%, at least 85%, at least 90%, at least 95%, at least 98%, at least 99% or identical to the sequence set forth in SEQ ID NO: 136 or 143. A skilled person will understand that modifications can be made to the sequences according to SEQ ID NO: 136 or 143 without substantially changing the function or structure of the sequence including those described herein and known in the art.

### AAV Production

Various methods of producing rAAV particles and nucleic acid vectors are known (see, e.g., Zolotukhin et al., 2002 and U.S. Patent Publication Numbers US20070015238 and US20120322861, which are incorporated herein by reference and plasmids and kits available from ATCC and Cell Biolabs Inc.). In some embodiments, an expression cassette (e.g., a plasmid) comprising a gene of interest may be combined with one or more helper plasmids, e.g., that contain a rep gene (e.g., encoding Rep78, Rep68, Rep52 and Rep40) and a cap gene (encoding VP1, VP2, and VP3, including a modified VP region as described herein), and transfected into recombinant cells, called helper or producer cells such that the nucleic acid expression cassette is packaged or encapsidated inside the capsid and subsequently purified.

Non-limiting examples of mammalian helper cells include HEK293 cells, COS cells, HeLa cells, BHK cells, or CHO cells (see, e.g., ATCC®, CRL-1573™, ATCC® CRL 1651™, ATCC®, CRL-1650™, ATCC CCL-2, ATCC®, CCL-10™, or ATCC® CCL-61™). A helper cell may comprises rep and/or cap genes that encode the rep protein and/or cap proteins. In some embodiments, the packaging is performed in vitro. In some embodiments, a nucleic acid expression cassette (e.g., plasmid) containing the gene of interest is combined with one or more helper plasmids, e.g., that contain a rep gene a first serotype and a cap gene of the same serotype or a different serotype, and transfected into helper cells such that the rAAV particle is packaged. In some embodiments, the one or more helper plasmids include a first helper plasmid comprising a rep gene and a cap gene, and a second helper plasmid comprising one or more of the following helper genes: E1a gene, E1b gene, E4 gene, E2a gene, and VA gene. Helper genes are genes that encode helper proteins E1a, E1b, E4, E2a, and VA. Helper plasmids, and methods of making such plasmids, are known in the art and commercially available (see, e.g., pDF6, pRep, pDM, PDG, pDP1rs, pDP2rs, pDP3rs, pDP4rs, pDP5rs, pDP6rs, pDG (R484E/R585E), and pDP8 e.g., from Vector Biolabs; Cellbiolabs; Agilent Technologies; and Addgene. Plasmids that encode wild-type AAV coding regions for specific serotypes are also known and available. For example, pSub201 is a plasmid that comprises the coding regions of the wild-type AAV2 genome (Samulski et al., 1987.

ITR sequences and plasmids containing ITR sequences are known in the art and are commercially available (see, e.g., products and services available from Vector Biolabs; Cellbiolabs; Agilent Technologies and Addgene. Methods for large-scale production of AAV using a her-pesvirus-based system are also known. See for example. Clement et al., 2009. Methods of producing exosome-associated AAV, which can be more resistant to neutralizing anti-AAV antibodies, are also known.

Methods for producing and using pseudotyped rAAV vectors are also known in the art (see, e.g., Duan et al., 2001; Halbert et al., 2000; Zolotukhin et al., 2002; and Auricchio et al., 2001.

### Gene of Interest

The AAV particles described herein or compositions thereof may be used for delivering a gene of interest to a target cell, tissue or organ for the treatment of a subject in need thereof. In an example, the gene of interest may be a therapeutic molecule that is known to have utility in the treatment of a disease, disorder or condition. In another example, the therapeutic molecule may be a gene that encodes a RNA or protein of interest. It is envisaged that therapeutic molecules of the present invention will have a therapeutic effect in a cell, tissue or organ to which the therapeutic molecule is delivered. In the case of treating an ocular disorder, the therapeutic effect of a therapeutic molecule may be to inhibit inflammation and/or angiogenesis in a target cell or tissue. Therapeutic molecules encompassed by the present disclosure are not particularly limited so long as they can be expressed from an expression cassette disclosed herein or translated from a nucleic acid expressed from the same.

As used herein, a “nucleotide,” “polynucleotide” or “nucleic acid” or “nucleic acid molecule” means a polymer of nucleotides and includes genomic DNA, mRNA, CRNA, and cDNA, RNA, siRNA, shRNA and hpRNA. A given polynucleotide may be of cellular, genomic or synthetic origin, for example made on an automated synthesizer, and may be combined with carbohydrate, lipids, protein or other materials, labelled with fluorescent or other groups, or attached to a solid support to perform a particular activity defined herein, or comprise one or more modified nucleotides not found in nature, known to those skilled in the art. The polymer may be single-stranded, essentially double-stranded or partly double-stranded. Basepairing refers to standard basepairing between nucleotides, including G: U basepairs. “Complementary” means two polynucleotides are capable of basepairing (hybridizing) along part of their lengths, or along the full length of one or both.

The polynucleotides or nucleic acid sequences of the present application may be deoxyribonucleic acid (DNA) sequences or ribonucleic acid (RNA) sequences and may include naturally occurring bases including adenine, guanine, cytosine, thymidine and uracil. The sequences may also contain modified bases. Examples of such modified bases include aza and deaza adenine, guanine, cytosine, thymidine and uracil; and xanthine and hypoxanthine. The nucleic acid can be either double stranded or single stranded, and represents the sense or antisense strand. Further, the term “nucleic acid” includes the complementary nucleic acid sequences.

The term “nucleic acid molecule” or its derivatives, as used herein, is intended to include unmodified DNA or RNA or modified DNA or RNA. For example, it may be useful for the nucleic acid molecules of the disclosure to be composed of single- and double-stranded DNA, DNA that is a mixture of single- and double-stranded regions, single- and double-stranded RNA, and RNA that is a mixture of single- and double stranded regions, hybrid molecules comprising DNA and RNA that may be single stranded or, more typically double-stranded or a mixture of single- and double-stranded regions. In addition, it may be useful for the nucleic acid molecules to be composed of triple stranded regions comprising RNA or DNA or both RNA and DNA. The nucleic acid molecules of the disclosure may also contain one or more modified bases or DNA or RNA backbones modified for stability or for other reasons. “Modified” bases include, for example, tritiated bases and unusual bases such as inosine. A variety of modifications can be made to DNA and RNA; thus “nucleic acid molecule” encompasses chemically, enzymatically, or metabolically modified forms. The term “polynucleotide” shall have a corresponding meaning.

The term “isolated polynucleotide” means a polynucleotide which has generally been separated from the polynucleotide sequences with which it is associated or linked in its native state, if the polynucleotide is found in nature. Preferably, the isolated polynucleotide is at least 90% free from other components with which it is naturally associated, if it is found in nature. In an embodiment, the polynucleotide is not naturally occurring, for example by covalently joining two shorter polynucleotide sequences in a manner not found in nature (chimeric polynucleotide).

A “chimeric gene” refers to any gene that comprises covalently joined sequences that are not found joined in nature. Typically, a chimeric gene comprises regulatory and transcribed or protein coding sequences that are not found together in nature. Accordingly, a chimeric gene may comprise regulatory sequences and coding sequences that are derived from different sources, or regulatory sequences and coding sequences derived from the same source but arranged in a manner different than that found in nature. The term “endogenous” is used herein to refer to a substance that is normally present or produced in an unmodified organism at the same developmental stage as the organism under investigation. An “endogenous gene” refers to a native gene in its natural location in the genome of an organism. As used herein, “recombinant nucleic acid molecule”, “recombinant polynucleotide” or variations thereof refer to a nucleic acid molecule which has been constructed or modified by recombinant DNA/RNA technology. The terms “foreign polynucleotide” or “exogenous polynucleotide” or “heterologous polynucleotide” and the like refer to any nucleic acid which is introduced into the genome of a cell by experimental manipulations.

Foreign or exogenous genes may be genes that are inserted into a non-native organism or cell, native genes introduced into a new location within the native host, or chimeric genes. Alternatively, foreign or exogenous genes may be the result of editing the genome of the organism or cell, or progeny derived therefrom. A “transgene” is a gene that has been introduced into the genome by a transformation procedure.

The term “genetically engineered”, “genetically modified”, “genetic modification” or variants thereof refers to any genetic manipulation by man and includes introducing genes into cells by transformation or transduction, gene editing, cisgenesis, mutating genes in cells and altering or modulating the regulation of a gene in a cell or organisms to which these acts have been done or their progeny and so on.

Furthermore, the term “exogenous” in the context of a polynucleotide (nucleic acid) refers to the polynucleotide when present in a cell that does not naturally comprise the polynucleotide. The cell may be a cell which comprises a non-endogenous polynucleotide resulting in an altered amount of production of the encoded polypeptide, for example an exogenous polynucleotide which increases the expression of an endogenous polypeptide, or a cell which in its native state does not produce the polypeptide. Increased production of a polypeptide of the invention is also referred to herein as “over-expression”. An exogenous polynucleotide of the invention includes polynucleotides which have not been separated from other components of the transgenic (recombinant) cell, or cell-free expression system, in which it is present, and polynucleotides produced in such cells or cell-free systems which are subsequently purified away from at least some other components. The exogenous polynucleotide (nucleic acid) can be a contiguous stretch of nucleotides existing in nature or comprise two or more contiguous stretches of nucleotides from different sources (naturally occurring and/or synthetic) joined to form a single polynucleotide. Typically, such chimeric polynucleotides comprise at least an open reading frame encoding a polypeptide of the invention operably linked to a promoter suitable of driving transcription of the open reading frame in a cell of interest.

The % identity of a polynucleotide is determined by GAP (Needleman and Wunsch, 1970) analysis (GCG program) with a gap creation penalty=5, and a gap extension penalty=0.3 Preferably, the GAP analysis aligns two sequences over their entire length.

With regard to the defined polynucleotides, it will be appreciated that % identity figures higher than those provided above will encompass preferred embodiments. Thus, where applicable, in light of the minimum % identity figures, it is preferred that the polynucleotide comprises a polynucleotide sequence which is at least 50%, at least 60%, more preferably at least 70%, more preferably at least 75%, more preferably at least 80%, more preferably at least 85%, more preferably at least 90%, more preferably at least 91%, more preferably at least 92%, more preferably at least 93%, more preferably at least 94%, more preferably at least 95%, more preferably at least 96%, more preferably at least 97%, more preferably at least 98%, more preferably at least 99%, more preferably at least 99.1%, more preferably at least 99.2%, more preferably at least 99.3%, more preferably at least 99.4%, more preferably at least 99.5%, more preferably at least 99.6%, more preferably at least 99.7%, more preferably at least 99.8%, and even more preferably at least 99.9% identical to the relevant nominated SEQ ID NO.

When referring to polynucleotide identity herein, it will be understood that a given sequence identity is in reference to the open reading frame sequence.

In a further embodiment, the present invention relates to polynucleotides which are substantially identical or identical to those specifically described herein. As used herein, with reference to a polynucleotide the term “substantially identical” means the substitution of one or a few (for example 2, 3, or 4) nucleotides whilst maintaining at least one activity of the native protein encoded by the polynucleotide. In addition, this term includes the addition or deletion of nucleotides which results in the increase or decrease in size of the encoded native protein by one or a few (for example 2, 3, or 4) amino acids whilst maintaining at least one activity of the native protein encoded by the polynucleotide.

The present invention also relates to the use of oligonucleotides, for instance in methods of screening for a polynucleotide of, or encoding a polypeptide of, the invention. As used herein, “oligonucleotides” are polynucleotides up to 50 nucleotides in length. The minimum size of such oligonucleotides is the size required for the formation of a stable hybrid between an oligonucleotide and a complementary sequence on a nucleic acid molecule of the present invention. They can be RNA, DNA, or combinations or derivatives of either. Oligonucleotides are typically relatively short single stranded molecules of 10 to 30 nucleotides, commonly 15-25 nucleotides in length. When used as a guide for genome editing, probe or as a primer in an amplification reaction, the minimum size of such an oligonucleotide is the size required for the formation of a stable hybrid between the oligonucleotide and a complementary sequence on a target nucleic acid molecule. Preferably, the oligonucleotides are at least 15 nucleotides, more preferably at least 18 nucleotides, more preferably at least 19 nucleotides, more preferably at least 20 nucleotides, more preferably at least 22 nucleotides, even more preferably at least 25 nucleotides in length. Oligonucleotides of the present invention used as a probe are typically conjugated with a label such as a radioisotope, an enzyme, biotin, a fluorescent molecule or a chemiluminescent molecule.

As those skilled in the art would be aware, the sequence of the oligonucleotide primers described herein can be varied to some degree without effecting their usefulness for the methods of the invention. A “variant” of an oligonucleotide disclosed herein (also referred to herein as a “primer” or “probe” depending on its use) useful for the methods of the invention includes molecules of varying sizes of, and/or are capable of hybridising to the genome close to that of, the specific oligonucleotide molecules defined herein. For example, variants may comprise additional nucleotides (such as 1, 2, 3, 4, or more), or less nucleotides as long as they still hybridise to the target region. Furthermore, a few nucleotides may be substituted without influencing the ability of the oligonucleotide to hybridise the target region. In addition, variants may readily be designed which hybridise close (for example, but not limited to, within 50 nucleotides or within 100 nucleotides) to the region of the genome where the specific oligonucleotides defined herein hybridise.

The present invention includes oligonucleotides that can be used as, for example, guides for RNA-guided endonucleases, probes to identify nucleic acid molecules, or primers to produce nucleic acid molecules. Probes and/or primers can be used to clone homologues of the polynucleotides of the invention from other species. Furthermore, hybridization techniques known in the art can also be used to screen genomic or cDNA libraries for such homologues.

Nucleotide sequences encoding a recombinant AAV particle described herein including the modified capsid protein of the invention may also be defined by their capability to hybridise with a nucleotide sequence under moderate, or preferably under stringent hybridisation conditions. Stringent hybridisation conditions are herein defined as conditions that allow a nucleic acid sequence of at least about 25, preferably about 50 nucleotides, 75 or 100 and most preferably of about 200 or more nucleotides, to hybridise at a temperature of about 65° C. in a solution comprising about 1 M salt, preferably 6×SSC or any other solution having a comparable ionic strength, and washing at 65° C. in a solution comprising about 0.1 M salt, or less, preferably 0.2×SSC or any other solution having a comparable ionic strength. In an embodiment, stringent conditions are those that (1) employ low ionic strength and high temperature for washing, for example, 0.015 M NaCl/0.0015 M sodium citrate/0.1% NaDodSO4 at 50° C.; (2) employ during hybridisation a denaturing agent such as formamide, for example, 50% (vol/vol) formamide with 0.1% bovine serum albumin, 0.1% Ficoll, 0.1% polyvinylpyrrolidone, 50 mM sodium phosphate buffer at pH 6.5 with 750 mM NaCl, 75 mM sodium citrate at 42° C.; or (3) employ 50% formamide, 5×SSC (0.75 M NaCl, 0.075 M sodium citrate), 50 mM sodium phosphate (pH 6.8), 0.1% sodium pyrophosphate, 5×Denhardt's solution, sonicated salmon sperm DNA (50 g/ml), 0.1% SDS and 10% dextran sulfate at 42° C. in 0.2×SSC and 0.1% SDS. Preferably, the hybridisation is performed overnight, i.e. at least for 10 hours and preferably washing is performed for at least one hour with at least two changes of the washing solution. These conditions will usually allow the specific hybridisation of sequences having about 90% or more sequence identity.

Moderate conditions are herein defined as conditions that allow a nucleic acid sequences of at least 50 nucleotides, preferably of about 200 or more nucleotides, to hybridise at a temperature of about 45° C. in a solution comprising about 1 M salt, preferably 6×SSC or any other solution having a comparable ionic strength, and washing at room temperature in a solution comprising about 1 M salt, preferably 6×SSC or any other solution having a comparable ionic strength. Preferably, the hybridisation is performed overnight, i.e. at least for 10 hours, and preferably washing is performed for at least one hour with at least two changes of the washing solution. These conditions will usually allow the specific hybridisation of sequences having up to 50% sequence identity. The person skilled in the art will be able to modify these hybridisation conditions in order to specifically identify sequences varying in identity between 50% and 90%.

The term ‘transgene’ as used herein refers to particular nucleic acid sequences encoding a polypeptide or a portion of a polypeptide to be expressed in a cell into which the nucleic acid sequence is introduced. However, it is also possible that transgenes are expressed as RNA, typically to control (e.g., lower) the amount of a particular polypeptide in a cell into which the nucleic acid sequence is inserted. The transgene may be homologous or heterologous to the promoter (and/or to the animal, in particular mammal, in which it is introduced, e.g. in cases where the nucleic acid expression cassette is used for gene therapy).

The transgene may be a full-length cDNA or genomic DNA sequence, or any fragment, subunit or mutant thereof that has at least some biological activity that is effective for the treatment of a condition, disease or disorder in a subject in need thereof. In particular, the transgene may be a minigene, i.e. a gene sequence lacking part, most or all of its intronic sequences. The transgene thus optionally may contain intron sequences. Optionally, the transgene may be a hybrid nucleic acid sequence, i.e., one constructed from homologous and/or heterologous cDNA and/or genomic DNA fragments.

Where a ‘mutant form’ is contemplated, a nucleic acid sequence may contain one or more nucleotides that are different from the wild-type or naturally occurring sequence, i.e., the mutant nucleic acid sequence contains one or more nucleotide substitutions, deletions, and/or insertions. The nucleotide substitution, deletion, and/or insertion can give rise to a gene product (i.e. e., protein or nucleic acid) that is different in its amino acid/nucleic acid sequence from the wild type amino acid/nucleic acid sequence. Preparation of such mutants is known in the art. In the context of the present invention, any nucleotide substitutions, deletions, and/or insertions introduced into the transgene nucleotide sequence do not significantly adversely affect the function of a therapeutic effect of the transgene, but may enhance it.

In an example, the AAV particle or composition thereof comprises a nucleic acid sequence that encodes a therapeutic polypeptide. For example, a nucleic acid sequence can be expressed from an AAV particle defined herein and translated by cellular machinery to produce a therapeutic polypeptide.

Exemplary therapeutic polypeptides include binding proteins such as immunoglobulin, antibodies and antigenic binding fragments. For example, therapeutic polypeptides include single chain Fv fragment (scFv), dimeric scFv (di-scFv), (scFv) n, scFv or di-scFv linked to a constant region of an antibody, Fc or a heavy chain constant domain (CH) 2 and/or CH3, diabodies, triabodies, tetrabodies, Fab, F(ab′) 2 and antibodies. In an example, the therapeutic polypeptide is an antibody or TRAP molecule. Examples of such therapeutic molecules include ranibizumab, bevacizumab and aflibercept.

In another example, the therapeutic polypeptide comprises an antigen binding site of an antibody.

In another example, the therapeutic molecule can be an inhibitory oligonucleotide. Exemplary inhibitory oligonucleotides include isolated or synthetic antisense RNA or DNA, siRNA or siDNA, miRNA, miRNA mimics, shRNA or DNA and Chimeric Antisense DNA or RNA. The term “antisense” as used herein means a sequence of nucleotides complementary to and therefore capable of binding to a coding sequence, which may be either that of the strand of a DNA double helix that undergoes transcription, or that of a messenger RNA molecule. The terms “short hairpin RNA” or “shRNA” refer to an RNA structure having a duplex region and a loop region. The term small interfering RNA (siRNA), sometimes known as short interfering RNA or silencing RNA, is a class of double-stranded RNA molecules, 20-25 base pairs in length. A siRNA that inhibits or prevents translation to a particular protein is indicated by the protein name coupled with the term siRNA. Thus a siRNA that interferes with the translation of VEGF is indicated by the expression “VEGF siRNA”. The term “microRNA” (abbreviated miRNA) is a small non-coding RNA molecule (containing about 22 nucleotides) found in plants, animals and some viruses, that functions in RNA silencing and post-transcriptional regulation of gene expression. The prefix “miR” is followed by a dash and a number, the latter often indicating order of naming. Different miRNAs with nearly identical sequences except for one or two nucleotides are annotated with an additional lower case letter. Numerous miRNAs are known in the art (miRBase V.21 nomenclature; Kozomara et al., 2014; Griffiths-Jones, 2004). In another example, an inhibitory oligonucleotide encompassed by the present disclosure inhibits the activity of one or more miRNAs. Various species are suitable for this purpose. Examples include antagomirs, interfering RNA, ribozymes, miRNA sponges and miR-masks. The term “antagomir” is used in the context of the present disclosure to refer to chemically modified antisense oligonucleotides that bind to a target miRNA and inhibit miRNA function by preventing binding of the miRNA to its cognate gene target.

In an example, the therapeutic molecule is an “anti-angiogenic molecule”. The term “anti-angiogenic molecule” is used in the context of the present disclosure to refer to molecules expressed from an AAV particle defined herein which inhibit the development of blood vessels, e.g., inhibit angiogenesis, endothelial cell growth, stability of blood vessels, and/or vasculogenesis. Anti-angiogenic molecules encompassed by the present disclosure include polynucleotide(s), polypeptide(s), antibod (ies) or conjugates or fusion proteins thereof. Exemplary anti-angiogenic molecules include inhibitors of VEGF and members of the VEGF family, PIGF, PDGF family, fibroblast growth factor family (FGFs), TIE ligands (Angiopoietins), ephrins, ANGPTL3 and ANGPTL4. In other examples, anti-angiogenic molecules inhibit growth hormones such as insulin-like growth factor-I (IGF-I), VIGF, epidermal growth factor (EGF), CTGF and members of the CTGF family, TGF-α and TGF-β. Other examples of anti-angiogenic molecules include antibodies to VEGF, antibodies to VEGF receptors and angiogenesis inhibitors such as angiostatin, endostatin and fusions thereof. Accordingly, in an example, the therapeutic molecule comprises endostatin and/or angiostatin. In another example, the therapeutic molecule is a fusion of one or more of the molecules described herein. For example, the therapeutic molecule can comprise a fusion of endostatin and angiostatin.

In an example, the therapeutic molecule is an anti-inflammatory molecule. In an example, the anti-inflammatory molecule is an interleukin such as IL-10, IL-4, IL-6, IL-11 or IL-13. For example, the anti-inflammatory molecule can be IL-10. In another example, the anti-inflammatory molecule is interleukin IL-1 receptor antagonist (IL-1RA), or a fusion of IL-10 and IL-1RA. In other examples, the anti-inflammatory molecule inhibits a pro-inflammatory cytokine such as IL-1, tumour necrosis factor alpha (TNF-α) or IL-18. In other examples, anti-inflammatory molecules increase the production or number of CD14+CD16+ cells in a subject, reduce IL-6 levels, reduce TNF-alpha levels and/or increase IL-10 levels.

In another example, the therapeutic molecule is a neurotrophic factor. Neurotrophic factors are thought to be responsible for the maturation of developing neurons and for maintaining adult neurons. In this regard, neurotrophic factors can be used to inhibit or reverse neural cell degeneration and death. Examples of neurotrophic factors include, for example, brain-derived neurotrophic factor, nerve growth factor, transforming growth factors, glial cell-line derived neurotrophic factor, neurotrophin 3, neurotrophin 4/5, and interleukin 1-B.

In another example, the therapeutic molecule is cytotoxic to cancer cells. In other examples, the therapeutic molecule inhibits one or more of Nuclear factor-kappa B (NFκB), C-kit (CD117, stem cell-factor receptor), Heat shock protein 90 (Hsp90), Ras-Raf mitogen-activated protein kinase (MEK) pathway, Bcl-2, IL-2 or TNF-alpha (e.g., anti-inflammatory soluble TNF-R).

### Treatment and Prevention

In an aspect of the invention, there are provided methods for treating or preventing a disease, condition or disorder in a subject comprising administering an AAV particle described herein or a composition thereof to the subject.

The terms “patient” and “subject” to be treated herein are used interchangeably and refer to patients and subjects of human or other mammal and includes any individual being examined or treated using the methods of the invention. Suitable mammals that fall within the scope of the invention include, but are not restricted to, primates, companion animals (i.e., cats and dogs) laboratory test animals (e.g., rabbits, mice, rats) and any other mammal that suffers from diseases, conditions or disorders that are receptive to gene therapy.

Where it is contemplated that the invention treats or prevents a disease, condition or disorder that is receptive to gene therapy, it will be understood that the AAV capsids, particles and AAV vectors of the invention have utility in a broad range of diseases, conditions or disorders. Whilst it is preferable that the AAV capsids, particles and AAV vectors are used to treat an ocular disorder, utility for the AAV capsids, particles and AAV vectors has been demonstrated in various cell types including retinal cells, melanoma cells and hepatic cells. In particular, increased transduction efficiency has been demonstrated for various cell types including retinal cells, melanoma cells and hepatic cells. It will therefore be understood that the therapeutic utility of the AAV capsids, particles and AAV vectors is not limited to ocular disorders.

In one example, the AAV capsids, particles and AAV vectors of the invention are directed to treating or preventing an ocular disorder. The term “ocular disorder” is used in the context of the present disclosure to refer to disorders and anomalies that affect the human eye and visual system. For example, ocular disorders include, but are not limited congenital, developmental, inflammatory, infectious, vascular, occlusive, angiogenic, degenerative, neoplastic, pre-neoplastic, iatrogenic, traumatic, glaucomatous, post-transplant complications, cataractous, and idiopathic diseases of the eye, retina, choroid or macula, or the systemic predispositions, associations or complications of these diseases (such as metastatic uveal melanoma or multisystem effects in an inherited gene dystrophy).

Other exemplary ocular disorders include diabetic retinopathy, cystoid macular oedema, clinically significant macular oedema, uveitis, iritis, giant cell arteritis, vasculitis, pars planitis, corneal transplant rejection, intraocular inflammation and lamellar corneal transplant rejection. Other examples of ocular disorders encompassed by the present disclosure include macular degeneration, diabetic retinopathy, cystoid macular oedema, clinically significant macular oedema, central retinal vein occlusion, branch retinal vein occlusion or ocular neovascularisation. For example, the ocular disorder can be an “intraocular neovascular disease”. The term, “intraocular neovascular disease” is used in the context of the present disclosure to refer to a disease characterized by ocular neovascularization. Examples of intraocular neovascular diseases include, but are not limited to, e.g., proliferative retinopathies, choroidal neovascularization (CNV), dry age-related macular degeneration (AMD), wet age-related macular degeneration (AMD), diabetic and other ischemia-related retinopathies, diabetic macular edema, pathological myopia, von Hippel-Lindau disease, histoplasmosis of the eye, Central Retinal Vein Occlusion (CRVO), corneal neovascularization and retinal neovascularization. For example, the methods of the present disclosure encompass treating wet AMD. In another example, the methods of the present disclosure encompass treating CNV.

In an example, the methods of the present disclosure encompass inhibition of endothelial cell growth in the retina. For example, the methods of the present disclosure encompass inhibition of endothelial cell growth in the sub-retinal pigment epithelium and/or sub retinal space.

In another example, the ocular disorder is an infection.

In another example, the ocular disorder is a cancer. In one example, the cancer is uveal melanoma, ciliary body melanoma, iris melanoma, choroidal melanoma, intraocular lymphoma, retinoblastoma, or medulloepithelioma, choroidal hemangioma, choroidal metastasis, conjunctival Kaposi's sarcoma, malignant conjunctival tumor, orbital or lacrimal gland lymphoma, lymphoma of the conjunctiva, conjunctival melanoma or primary acquired melanosis with atypia, malignant tumour of the orbit, malignant tumour of the lacrimal gland, pigmented conjunctival tumour, squamous carcinoma of the conjunctiva, intraepithelial neoplasia of the conjunctiva or ocular surface squamous neoplasia. In one example, the cancer is uveal melanoma.

In another example, the ocular disorder is choroidal nevus, choroidal osteoma, nevus of Ota, conjunctival naevus, epibulbar dermoid, benign tumors of the lacrimal gland, benign tumours of the orbit, manifestations of thyroid ophthalmopathy, pingueculum, or pterygium.

In another example, the ocular disorder is Leber congenital amaurosis with RPE65 or an ocular disorder caused by RPE65 mutations. In another example, the ocular disorder is retinitis pigmentosa caused by a mutation in MERTK or RPGR or PDE6B or RLBP1 or other genes, or another disease caused by a mutation in MERTK or RPGR or PDE6B or RLBP1. In another example, the ocular disorder is choroideremia or an ocular disorder caused by mutations in CHM. In another example, the ocular disorder is Achromatopsia or an ocular disorder caused by mutations in the CNGA3, CNGB3, GNAT2, PDE6C, PDE6H and ATF6 genes. In another example, it is X-linked retinoschisis or an ocular disorder caused by mutations in RS1. In another example, the ocular disorder is Leber's Hereditary Optic Neuropathy. In another example, the ocular disorder is a complication of transplant.

In an example, the methods described herein comprise administering an AAV vector or particle described herein. For example, the above referenced methods can comprise administering an AAV vector or particle which comprises an expression cassette comprising a kill switch and a nucleic acid encoding a therapeutic molecule, wherein activation of the kill switch silences expression of the nucleic acid encoding the therapeutic molecule. In an example, the expression cassette further comprises a regulatable element operably linked to the nucleic acid encoding the therapeutic molecule, wherein activity of the regulatable promoter is regulated by administration of a regulator compound to the subject. In an example, the expression cassette further comprises a constitutive promoter operably linked to a regulator compound-binding molecule which binds the regulator compound, wherein upon binding the regulator compound, the regulator binding-polypeptide regulates expression of the therapeutic molecule.

In another example, the regulator compound-binding molecule and therapeutic molecule are expressed from separate expression cassettes.

The AAV capsids, particles and AAV vectors described herein may also be administered in combination with an additional treatment.

In one example, the additional treatment may include surgery, lens replacement with an intraocular lens, laser surgery or medication.

In another example, the additional treatment may include an immunosuppressant to prevent an adverse immune response to the AAV particle or vector in the subject. Where the administration of an immunosuppressant is contemplated, the treatment may be provided prior to the administration, or concurrently with the administration of the AAV particle or vector and may be temporary for instance, one, two, three, four or more weeks such that sufficient immunosuppression is achieved.

In an embodiment, the subject to be treated exhibits one or more symptoms of a disease or disorder associated with an ocular disorder described herein or known in the art.

For example, symptoms of an ocular disorder may include:
decreased peripheral vision;
decreased central vision;
decreased night vision;
loss of colour perception.
   Where the ocular disease is age-related macular degeneration, non-limiting examples of symptoms include one or more of:
blurry or hazy vision;
difficulty in adapting eyesight from low to bright light;
dark or blank spots in vision;
visual distortion.

Thus, a positive response to treatment with a AAV particle or vector described herein may include amelioration of one of more of the above-described symptoms or other symptoms known in the art. For instance, an individual having a positive response to treatment with an AAV vector administered as a result of the methods described herein may have a reduced blurry or hazy vision, reduced difficulty in adapting eyesight from low to bright light, reduced dark or blank spots in vision and/or reduced visual distortion. Alternatively, the symptoms may have disappeared altogether.

In one embodiment, the subject has become symptomatic for the ocular disorder. In another embodiment, the subject has 10% or more photoreceptor damage/loss. In another embodiment, the subject has 20% or more photoreceptor damage/loss. In another embodiment, the subject has 30% or more photoreceptor damage/loss. In another embodiment, the subject has 40% or more photoreceptor damage/loss. In another embodiment, the subject has 50% or more photoreceptor damage/loss. In another embodiment, the subject has 60% or more photoreceptor damage/loss. In another embodiment, the subject has 70% or more photoreceptor damage/loss. In another embodiment, the subject has 80% or more photoreceptor damage/loss. In another embodiment, the subject has 90% or more photoreceptor damage/loss. In another embodiment, the subject's bipolar cell circuitry to ganglion cells and optic nerve remains intact.

“Therapeutically effective amount” is used herein to denote any amount of a composition or AAV vector defined herein which is capable of reducing one or more of the symptoms associated with a disease, condition or disorder. A single administration of the therapeutically effective amount may be sufficient, or they may be applied repeatedly over a period of time, such as several times a day for a period of days or weeks. The amount of the active ingredient will vary with the conditions being treated, the stage of advancement of the condition, the age and type of host, and the type and concentration of the formulation being applied. Appropriate amounts in any given instance will be readily apparent to those skilled in the art or capable of determination by routine experimentation.

The terms “treatment” or “treating” of a subject includes the application or administration of a drug or compound with the purpose of delaying, slowing, stabilizing, curing, healing, alleviating, relieving, altering, remedying, less worsening, ameliorating, improving, or affecting the disease or condition, the symptom of the disease or condition, or the risk of (or susceptibility to) the disease or condition. The term “treating” refers to any indication of success in the treatment or amelioration of an injury, pathology or condition, including any objective or subjective parameter such as abatement; remission; lessening of the rate of worsening; lessening severity of the disease; stabilization, diminishing of symptoms or making the injury, pathology or condition more tolerable to the subject; slowing in the rate of degeneration or decline; making the final point of degeneration less debilitating; or improving a subject's physical or mental well-being.

As used herein, “preventing” or “prevention” is intended to refer to at least the reduction of likelihood of the risk of (or susceptibility to) acquiring a disease or disorder (i.e., causing at least one of the clinical symptoms of the disease not to develop in a patient that may be exposed to or predisposed to the disease but does not yet experience or display symptoms of the disease). Biological and physiological parameters for identifying such patients are provided herein and are also well known by physicians. For example, in the case of age-related macular degeneration, a subject may have a family history of the disease, may be of an age that predisposes an individual to AMD or may have a history of smoking, obesity, high cholesterol or high blood pressure yet show any apparent symptoms of the disease. In this case, it is contemplated that the AAV vectors and compositions thereof described herein will have utility in the preventing the onset of one or more symptoms associated with AMD in the subject.

The present invention also contemplates methods for the identification or diagnosis of a subject requiring treatment or prevention of a disease, condition or disorder. Preferably the method comprises identifying a subject requiring treatment for an ocular disorder. Diagnosis as used herein refers to the determination that a subject or patient requires treatment or prevention of a disease, condition or disorder. The type of disease or disorder diagnosed according to the methods described herein may be any type known in the art or described herein and is preferably an ocular disorder.

In an embodiment, the step of identifying a subject requiring treatment or prevention of an ocular disorder comprises assessment of the eye via one or more or all of:
Angiography (e.g., fluorescein angiography or indocyanine green angiography);
Electroretinography;
Ultrasonography;
Pachymetry;
Optical Coherence Tomography;
Computed Tomography (CT) and Magnetic Resonance Imaging (MRI).

### Formulations

The AAV particles and other molecules described herein may be formulated as a pharmaceutical composition suitable for administration to a subject. Exemplary pharmaceutical compositions can comprise a pharmaceutically acceptable carrier, diluent or excipient. Depending upon the particular route of administration, a variety of acceptable carriers, known in the art may be used, as for example described in Remington's Pharmaceutical Sciences (Mack Publishing Co. N.J. USA, 1991).

Exemplary pharmaceutical compositions may also comprise pharmaceutically acceptable sterile aqueous or nonaqueous solutions, dispersions, suspensions, or emulsions, as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol, and the like), and suitable mixtures thereof, vegetable oils, and injectable organic esters such as ethyl oleate. Compositions may also contain adjuvants such as preservatives, wetting agents, emulsifying agents, and dispersing agents or antibacterial and antifungal agents.

Various formulations have been developed to facilitate rAAV particle use. For example, for administration of an injectable aqueous solution of rAAV particles, the solution may be suitably buffered, if necessary, and the liquid diluent first rendered isotonic with sufficient saline or glucose. In some embodiments, a composition as provided herein comprises a plurality of any one or more of the recombinant AAV particles disclosed herein. USP grade carriers and excipients are particularly useful for delivery of rAAV particles to human subjects. Such compositions may further optionally comprise a liposome, a lipid, a lipid complex, a microsphere, a microparticle, a nanosphere, or a nanoparticle, or may be otherwise formulated for administration to the cells, tissues, organs, or body of a subject in need thereof. Methods for making such compositions are well known and can be found in, for example, Remington: The Science and Practice can be found in, for example, Remington: The Science and Practice of Pharmacy, 22^{nd} edition. Pharmaceutical Press, 2012.

In another example, the AAV particles can be incorporated into slow release or targeted delivery systems. Exemplary slow release systems include polymer matrices, liposomes, and microspheres. Liposomes may be biodegradable and amphiphilic drug delivery systems, which may be formulated using phospholipids and cholesterol. Microspheres may be formulated using biodegradable and biocompatible polymers.

In an example, regulator compounds are provided in a topical formulation. For example, regulator compounds can be provided as an eye drop formulation. Suitable exemplary eye drop formulations include solutions, suspensions, ointments, gels or foams. In an example, the eye drop formulation comprises a regulator compound defined herein and a suitable carrier. Exemplary carriers include saline solution, water polyethers such as polyethylene glycol, polyvinyls such as polyvinyl alcohol and povidone, cellulose derivatives such as methylcellulose and hydroxypropyl methylcellulose, petroleum derivatives such as mineral oil and white petrolatum, animal fats such as lanolin, polymers of acrylic acid such as carboxypolymethylene gel, vegetable fats such as peanut oil and polysaccharides such as dextrans, and glycosaminoglycans such as sodium hyaluronate and salts such as sodium chloride and potassium chloride.

In an example the AAV vector or particle thereof is present in or on a device that allows controlled or sustained release of the AAV vector or particle thereof, such as an ocular sponge, meshwork, mechanical reservoir, or mechanical implant. Implants (see, e.g., U.S. Pat. Nos. 5,443,505, 4,853,224 and 4,997,652), devices (see, e.g., U.S. Pat. Nos. 5,554,187, 4,863,457, 5,098,443 and 5,725,493), such as an implantable device, e.g., a mechanical reservoir, an intraocular device or an extraocular device with an intraocular conduit, or an implant or a device comprised of a polymeric composition are particularly useful for ocular administration of the AAV vector or particle thereof.

In an example, the AAV vector or particle thereof is formulated to enhance transduction efficiency, i.e., to enhance transduction of the AAV vector into a host cell. Suitable compositions are further described in U.S. Pat. Nos. 6,225,289 and 6,514,943.

### Administration and Dosing

In an example, an AAV particle or composition thereof defined herein is administered to a subject. In an example, the composition is administered via intravitreal injection. In another example, the composition is administered via subretinal injection. In one example, a vitrectomy is performed prior to subretinal injection. In another example, the composition is administered via subcutaneous injection. In another example, the composition is administered via intramuscular injection. In another example, the composition is administered via intravenous injection. In another example, the composition is administered as a food or drink composition. In other examples involving use of heat or light as regulator compounds, such compounds can be applied to the eye of a subject as required.

In an example a pharmaceutical composition comprising a AAV vector or particle thereof is administered via an ophthalmologic instrument for delivery to a specific region of an eye. Use of a specialized ophthalmologic instrument ensures precise administration of the AAV vector or particle thereof while minimizing damage to adjacent ocular tissue. Delivery of the AAV vector or particle thereof to a specific region of the eye also limits exposure of unaffected cells to the therapeutic molecule, thereby reducing the risk of side effects. One example of such an ophthalmologic instrument is a combination of forceps and subretinal needle or sharp bent cannula.

Where it is desirable to use the regulatable elements disclosed in WO2019144186, the entire contents of which is hereby incorporated by reference, expression of a therapeutic molecule from the AAV vector or particle thereof may be regulated via administration of a regulator compound to the subject. Expression of the therapeutic molecule may be subsequently silenced by administering a kill switch activator to the subject or by administering a AAV vector or particle thereof comprising a nucleic acid sequence encoding a kill switch activator. Each of the AAV vector or particle thereof, regulator compound and kill switch activator can be administered to a subject as a pharmaceutical composition. The selection of administration route will depend on a variety of factors such as, for example, the host, immunogenicity of the AAV vector, the desired duration of therapeutic molecule production, and the like.

In one example, a pharmaceutical composition comprising an AAV vector or particle thereof is administered by intravitreal or subretinal injection, a pharmaceutical composition comprising a regulator compound(s) is subsequently administered topically by way of eye drops and a kill switch activator is subsequently administered topically and/or by intravitreal or subretinal injection. Where the use of eye drops is contemplated, the composition or part thereof can diffuse into the intraocular environment through the hydrophobic cornea.

In another example, a pharmaceutical composition is administered to the human retina via delivery into the vitreous (i.e., intravitreal administration). In the context of the present invention, this route of administration is preferable because it is simpler and safer whilst providing a greater volume of diffusion. The presence of AAV neutralising antibodies in the vitreous along with the physical barrier created by the inner limiting membrane of the retina are known to be hurdles in choosing this administration. The AAV particles or compositions thereof are particular advantageous when administering into the vitreous because they are associated with increased transduction efficiency and a reduced immune response. In another example, compositions can be administered orally. In another example, compositions can be administered intranasally.

One of skill in the art will appreciate that dosage and routes of administration can be selected to minimize loss of transgene expression due to a host's immune system. For example, for contacting ocular cells in vivo, it can be advantageous to administer to a host a null expression AAV vector (i.e., an AAV vector not comprising the nucleic acid sequence encoding the therapeutic molecule) prior to performing the methods described herein. Prior administration of null expression AAV vectors can serve to create an immunity (e.g., tolerance) in the host to the AAV vector or particle thereof, thereby decreasing the amount of AAV vector cleared by the host's immune system.

Compositions disclosed herein can also be administered systemically, such as, for example, by intravenous or intraperitoneal administration. This administration route is particularly suitable for administration of an immunosuppressant for suppressing any potential adverse immune response to an AAV particle, should it be required. Whilst it is envisaged that the AAV particles and compositions thereof can reduce immunogenicity in a subject, the use of an immunosuppressant may be used in conjunction to further suppress and/or optimise an immune response.

### Kits

Compositions according to the present disclosure can be provided in a kit or pack. For example, compositions disclosed herein may be packaged in a suitable container with written instructions for treating a disease, condition or disorder. In an example, compositions may be provided in single dose containers such as an eye dropper or pre-filled syringe for the treatment of an ocular disorder.

Kits of the present disclosure may comprise a therapeutic system. Such therapeutic systems can provide pre-programmed, unattended delivery of a composition at a rate, and for a time period, established to meet a specific therapeutic need. The system can be designed to minimize the patient's intervention and to optimize compliance with the prescribed regimen, for example.

In one example, the kit comprises an AAV vector or particle thereof) defined herein for treating a disease, condition or disorder.

### EXAMPLE

### General Materials and Methods

### Ethics Statement

Ethical clearance for this study was obtained from the Metro South Human Research Ethics Committee (HREC reference number: HREC/18/QPAH/189). Informed consent for research on tissue was obtained prior to death from donors or next of kin by the Queensland Eye Bank. Human eye cups were retrieved within 24 h of death and stored at 4° C. Before collection, donor samples were tested and negative for HIV and Hepatitis B and C. Eye cups used in these investigations were transduced with AAV 48-72 h post-mortem.

### Cell Culture

Human embryonic kidney 293 (HEK293) cells were cultured in RPMI1640 medium (Gibco), with 10% FBS (Gibco) and 1× Pen-strep (Gibco), in 5% CO2/95% humidified air at 37° C. Short tandem repeat profiling was used to confirm their identity and they were routinely tested for mycoplasma to ensure they were mycoplasma-free.

Plasmid Construction pAAV2/8 was created by cloning the AAV8 cap open reading frame (ORF) from AAV2rep-AAV8cap as a HindIII-PmeI restriction fragment between the HindIII and PmeI restriction sites of pAAV-RC2. pAAV2/8 p5 was created by cloning the p5 promoter from 7m8 (Addgene plasmid #64839) as an XbaI-HindIII restriction fragment between the XbaI and HindIII restriction sites of pAAV2/8. pAAV2/8 p5-2 was created by cloning the p5 promoter from Addgene plasmid #112864 as a PmeI-EcoRV restriction fragment into the PmeI restriction site of pAAV2/8 p5, downstream from the AAV8 cap ORF to act as an enhancer.

Y447F, T494V and Y733F amino acid substitutions were introduced into the AAV8 cap ORF by overlapping PCR using KOD Hot Start DNA polymerase. The sequences of the oligonucleotides used for PCR are listed in Table 6. AAV2rep-AAV8cap was a gift from John Chiorini. 7m8 was a gift from John Flannery and David Schaffer (Addgene plasmid #64839).

TABLE 6
Oligonucleotide primer sequences

| Primer name | Nucleotide sequence | Purpose |
|---|---|---|
| AAV8capF1058_BsiWI | TGCCGTACGTTCTCGGCTC | Introducing a |
| AAV8capY447F-Rev | CCGAGACAAGTAGAACAGGTACTGG | Y447F substitution |
| | TC | into the AAV8 cap |
| AAV8capY447F-For | GACCAGTACCTGTTCTACTTGTCTCG | gene. |
| | G | |
| AAV8capR1475_MluI | GAGACGCGTTGTTGGCGG | |
| AAV8capF1464_MluI | ACAACGCGTCTCAACGACAAC | Introducing a |
| AAV8capY733F-Rev | GGGTGAGGAAACGGGTG | Y733F substitution |
| AAV8capY733F-For | CACCCGTTTCCTCACCC | into the AAV8 cap |
| AAV8cap-Rev_PmeI | GCTGTTTAAACGAATTCGCCC | gene. |
| AAV8capT494V-For | ACAACGCGTCTCAACGGTAACCG | T494V substitution. |
| N590 F1 | CCAATCAGGCAAAGAACTGG | Introducing the |
| N590 R1 | CTTAAGATCACCGCGAGATCTGTTTT | BglII-RGD-AflII |
| | GCTGCTGCAAGTTATC | insertion |
| N590 F2 | AGATCTCGCGGTGATCTTAAGACGG | after N590. |
| | CTCCTCAAATTGGAAC | |
| N590 R2 | GCTGTTTAAACGAATTCGCCC | |
| N590 F3 | GATAACTTGCAGCAGCAAAACACCG | Creating random 7aa |
| | GT N₂₁ | insertions at BglII- |
| | GGTCTTTCGACGGCTCCTCAAATTGG | AflII. |
| | AAC | insertions at Bglll- |
| N590 R3 | GTTCCAATTTGAGGAGCCGT | AflII. |
| N590 R3 | GTTCCAATTTGAGGAGCCGT | |

Abbreviations: aa, amino acid; RGD, arginine-glycine-aspartic acid

### AAV2/8 Random Peptide Display Library Construction

Pairs of unique restriction sites for random peptide display library creation were added to pAAV2/8 p5-2 (Y447F, T494V, Y733F) by overlapping PCR using KOD Hot Start DNA polymerase and introduced either by restriction digestion and ligation or using an NEBuilder kit. These were BglII/AflII immediately 3′ of N590, respectively. A nine base pair sequence encoding an RGD peptide was included between each pair of restriction sites. AAV2 inverted terminal repeat (ITR) sequences were added by cloning the three p5-AAV2rep-AAV8cap (Y447F, T494V, Y733F mutant) N590 BglII/AflII cassettes between the XbaI and PmeI restriction sites of ITR-AAV6, which has an existing p5 promoter/enhancer 3′ of the PmeI site. To create a random peptide display plasmid library, oligonucleotides of the form: 21 nt homologous sequence-restriction site 1-N21-restriction site 2-21 nt homologous sequence, were synthesised, converted to double-stranded DNA using a complementary reverse primer, gel-purified and ligated with vector DNA (cut with the corresponding pair of restriction enzymes) by homologous recombination using an NEBuilder kit. ITR-containing plasmids and N590 plasmid library were used to transform, and were propagated in, NEB Stable E. coli cells. All other plasmids were propagated in NEB 5× cells. The sequences of the oligonucleotides used for PCR are listed in Table 6.

### AAV Production

AAV was produced from 70-80% confluent HEK293 cells in 10 cm diameter petri dishes, transfected with 5 μg pHelper, 2.5 μg pAAV2/8 plasmid and 2.5 μg ITR-CMV-tdTomato. Plasmids were first added to 500 μl DMEM, followed by 30 μl of linear polyethylenimine 25 000 kDa (Polysciences, US), mixed by pipetting up and down, incubated 10 minutes at room temperature, added dropwise to HEK293 cells in 10 ml of fresh medium, mixed by gentle rocking and incubated at 37° C. Forty-eight hours later, cells were lifted into the cell medium using a cell scraper, transferred to a 15-ml tube, collected by centrifugation at 400 g for 5 minutes, washed in 1 ml of PBS, transferred to a 1.5-ml tube and pelleted by centrifugation at 10,000 g for 2 minutes. 600 μl of PBS was added to the cell pellet and cells were lysed by three cycles of freezing in dry ice and thawing at 37° C. to release AAV. After the second thaw, cell debris was dispersed by gently flicking the tube. After a final centrifugation (7,000 g for 5 minutes followed by 10,000 g for 2 minutes), the supernatant containing AAV was transferred to a fresh 1.5-ml tube. AAV titration was performed by qPCR as described, https://www.addgene.org/protocols/aav-titration-qpcr-using-sybr-green-technology/. AAV random peptide display libraries were produced similarly but using 5 μg pHelper and 0.5 μg ITR-flanked random peptide display library plasmids.

### Library Selection Ex Vivo

To identify novel AAV variants, a five round selection method of initial AAV library was performed in human retina and subretinal tissue. In each step, AAV library were added to post-mortem retinal and RCS explants cultured in DMEM medium. At 12 hrs post transduction, medium was changed and at 48 h, genomic DNA was extracted from retinal and subretinal explants. Successful virions were PCR-amplified using following primers Forward 5′ GTTTCCCTGCAGACAATGCG 3′ and Reverse 5′ TCAAAATGGAGACCCTGCGT 3′. Consequently PCR products containing library-cap genes were recloned into the bacterial plasmid containing AAV-rep genes and ITR-2 and repackaged using AAV293 cells. Following the selection process, novel AAV variants were recloned into bacterial plasmid not containing ITR-2 elements, sequenced and AAV vector produced using AAV293 cells for downstream process.

### Statistical Analysis

Unless otherwise noted, data are presented as the mean and standard error of the mean. P<0.05 was considered statistically significant.

### Results

### Vector Optimisation to Improve AAV Titre

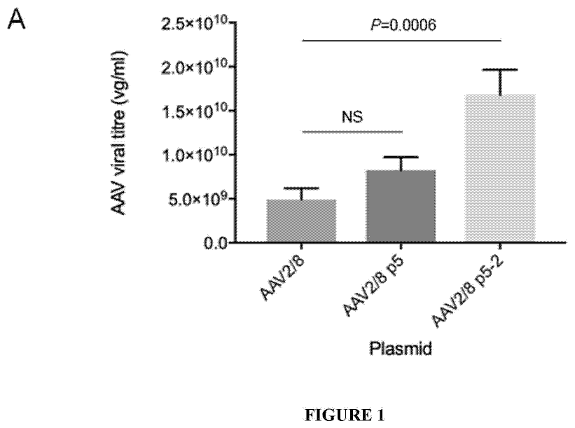

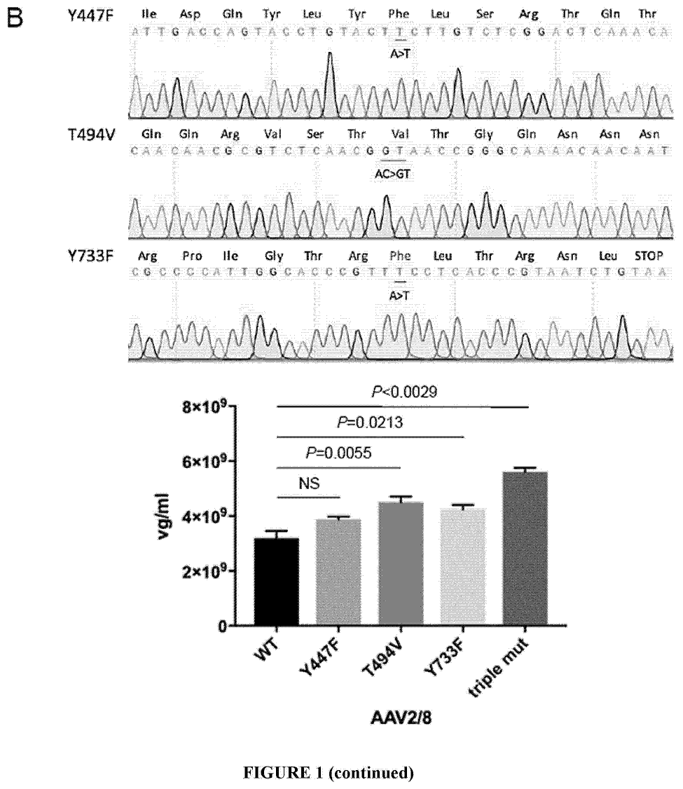

The inventors chose to investigate whether recombinant AAV2/8 could be modified for use as a gene therapy vector because of its unique preference for infecting human retina adjacent to blood vessels and because of the low prevalence in human vitreous of neutralising antibodies towards AAV8 (Halbert et al., 2006). Initially, the AAV8 cap gene was cloned into the pRC-AAV2 vector in place of the AAV2 cap gene to create the recombinant plasmid pAAV2/8. Transcription of the viral replication (rep) gene in this plasmid is from a bacterial promoter. To investigate whether AAV titre could be improved, AAV2 p5 promoter was added upstream of the AAV2 rep gene and the initiator methionine was mutated from ATG to ACG so that translation would begin at a downstream, in-frame methionine. This has the effect of preventing translation of the two largest Rep isoforms (p78 and p68) which negatively regulate AAV replication (Li et al., 1997). This resulted in a 1.67-fold increase in AAV titre (FIG. 1a). Additionally, the AAV2 p5 promoter was cloned downstream of the AAV8 cap gene to act as an enhancer element. This resulted in a further 1.74-fold increase in viral titre (FIG. 1a).

Substitution of several conserved tyrosine and threonine residues within the AAV2 cap gene with analogous (but non-phosphorylated) amino acids has been reported to increase AAV2 titre. We therefore investigated three of the most significant of these (Petrs-Silva et al., 2009; Kay et al., 2013—corresponding to Y447F, T494V and Y733F in AAV8—for their effect on AAV8 viral titre. All increased AAV titre, with T494V (1.41-fold) and Y733F (1.33-fold) being most effective (FIG. 1b). The combination of all three substitutions increased viral titre 1.75-fold (FIG. 1b). The plasmid map of the expression vector is shown in FIG. 2c. Together, these results show that changes to plasmid cis-acting regulatory sequences and conserved AAV8 capsid residues produced a vector that can enhance the titre of recombinant AAV2/8 produced using HEK293 packaging cells.

### Creation of AAV2/8 Random Display Peptide Libraries

Inserting short peptide sequences into loop IV of VP1-3, positioned such that they are exposed on the surface of the vector capsid at a site critical for viral attachment to the target cell, can alter recombinant AAV2/2 vector tropism (Nicklin et al., 2001; Grifman et al., 2001; Girod et al., 1999; Muller et al., 2003). The inventors reasoned that a similar strategy could be employed using AAV2/8, to screen for novel recombinant capsid structures that enhanced AAV-targeting of human retina. The AAV2/8 Y447F, T494V, Y733F triple mutant vector was engineered to display random peptide libraries within loop IV of the AAV2/8 capsid and flanked the p5 promoter and 3′ enhancer sequence with AAV2 inverted terminal repeats. The loop IV sites were chosen because of their location within the AAV2/8 capsid domains involved in receptor binding.

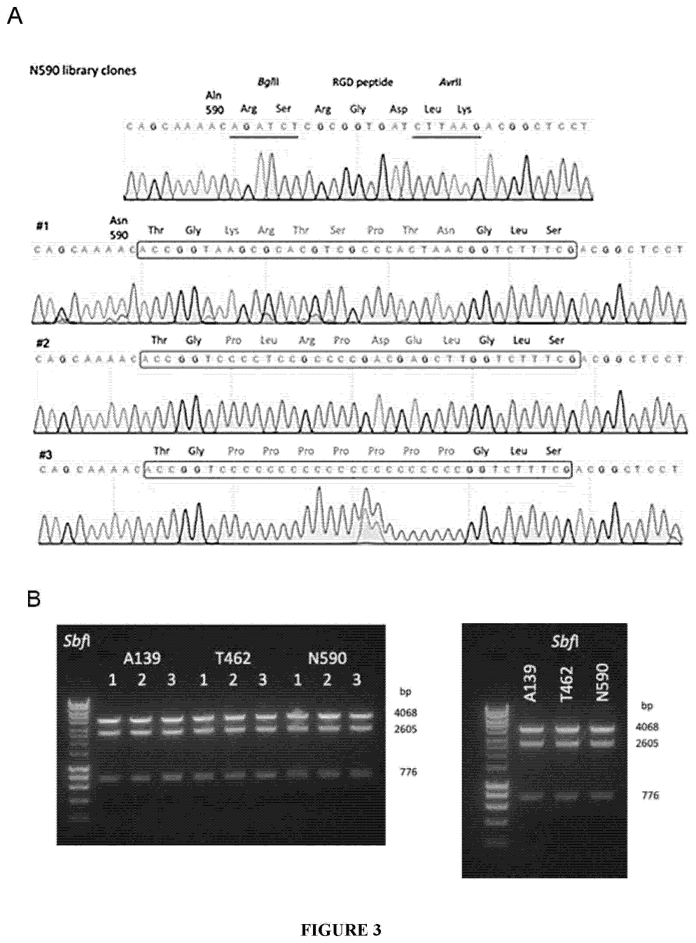

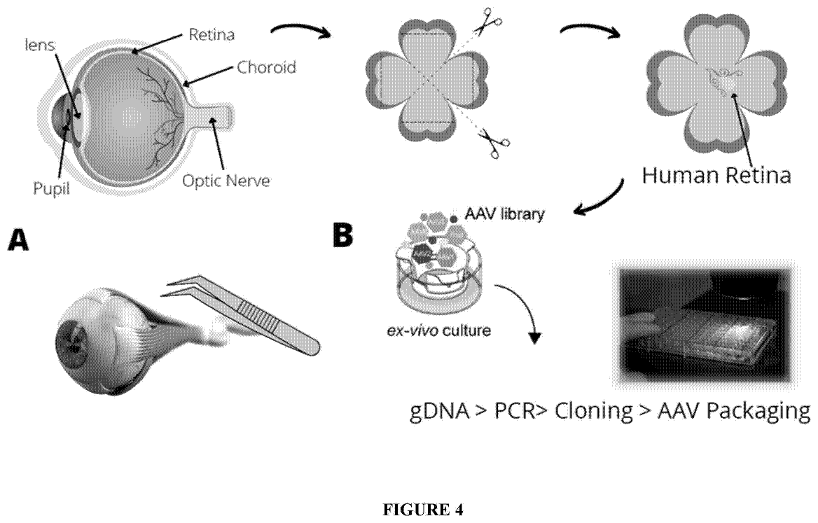

The library displays random seven-amino acid peptides flanked by two additional fixed amino acids at their N-terminus and three at their C-terminus (previously demonstrated to be compatible with peptide insertion) (Shi et al., 2001) (FIG. 2a). DNA sequencing of three clones from the library verified that different random peptides were encoded by each clone (FIG. 3a). Sbfl digestion of each clone confirmed the presence of ITRs (FIG. 3b). To produce AAV libraries, HEK293 packaging cells were transfected at a high cell:plasmid ratio, with the aim that individual packaging cells would take up only a single plasmid from the library, so that each AAV capsid only displayed the peptide sequence encoded by the ITR-flanked library cap gene it contained.

Identification of AAV8 Variants that Infect Human Retina from the Vitreous by Directed Evolution

To identify novel AAV variants, a five round selection method of initial AAV library was performed in human retina and subretinal tissue. In each step, AAV library were added to post-mortem retinal and RCS explants cultured in DMEM medium. At 12 hrs post transduction, medium was changed and at 48 h, genomic DNA was extracted from retinal and subretinal explants. Successful virions were PCR-amplified using following primers Forward 5′ GTTTCCCTGCAGACAATGCG 3′ and Reverse 5′ TCAAAATGGAGACCCTGCGT 3′. Subsequently, PCR products containing library-cap genes were recloned into the bacterial plasmid containing AAV-rep genes and ITR-2 and repackaged using AAV293 cells. Following the selection process, novel AAV variants were recloned into bacterial plasmid not containing ITR-2 elements and sequenced. Selected clones were AAV vector produced using triple transfection in AAV293 cells and used in functional assays.

### Functional Assessment of AAV8 Variants on the Immune System

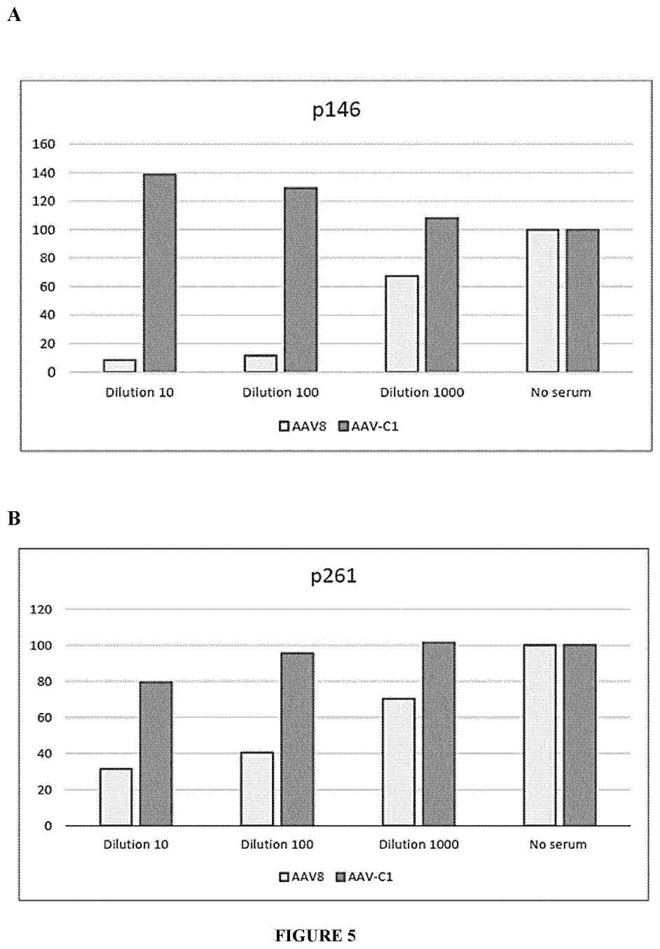

Cos-7, LnCap and Mel cells in 24-well and 96-well plates were transduced with recombinant AAV2/8 and AAV2/8 C1m1 vectors of the same titre using secNanoLuc luciferase as reporter. AAVs were diluted either in 250 μL of DMEM FBS-free medium or in 250 μL of serum of indicated dilutions (1/10, 1/100, 1/1000) obtained from matching donors and pre-incubated for 1 hour at 37° C. For AAV transduction cells were incubated for 16-24 hr at 37° C. in 5% CO₂ TC incubator. Medium containing secreted luciferase was collected, stored at −80° C. and measured at the end point. Luciferase expression was measured using a PHERAstar FSX (BMG-Labtech, Ortenberg, Germany) and the Nano-Glo luciferase assay system (Promega) according to the manufacturer's protocol. AAV2/8 C1m1 variant efficiently escaped neutralization by anti-AAV8 antibodies present in the serum of donors (serum samples p146 and p261 are shown), while unmodified AAV2/8 parental capsid was recognized by antibodies present in the serum (FIGS. 5A-B).

Comparison of Peptide Inserts into Loop IV of the AAV2/8 Capsid

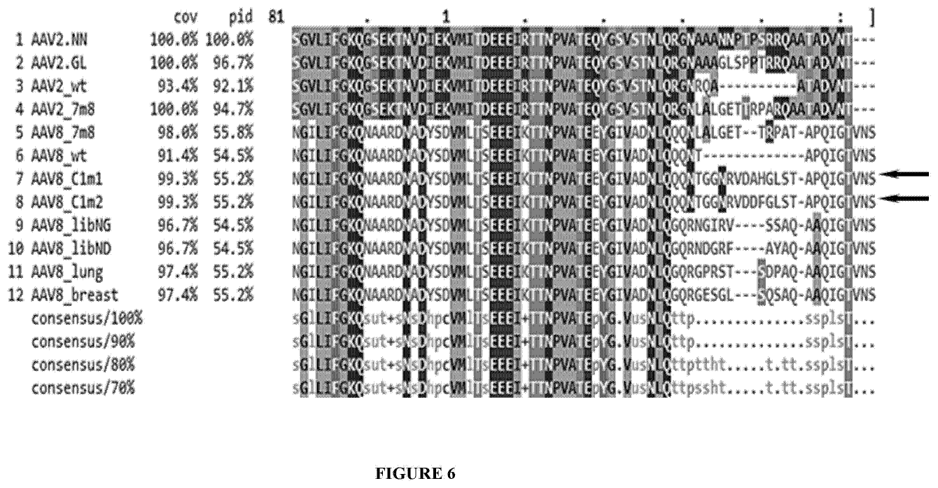

ClustalO alignment (https://www.ebi.ac.uk/Tools/msa/clustalo/) of peptide inserts into position N590 of capsid protein VP3 AAV8. MView1.63 was used for graphical representation of alignment (Brown et al., 1998). Inserts obtained after AAV8 N590 Library selection ex vivo (FIG. 6) were compared to published sequences of inserts at N590 of wild type AAV2 and AAV8 capsids: AAV-7m8 (Khabou et al., 2016), AAV2.NN, AAV2.GL (Pavlou et al., 2021), AAV8_lung, AAV8_breast (Büning and Srivastava, 2019), AAV8_libNG (Börner et al., 2020).

### Analysis of Reporter Fluorescence of Various AAV-GFP Optimised Clones

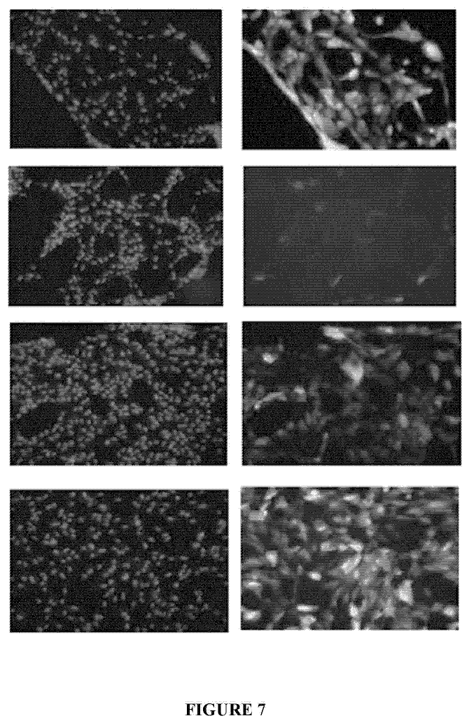

ARPE19 cells were transduced in triplicate with AAVs containing AAV-CMV-GFP reporter plasmid (Addgene #67634) and packaged with 7m8, AAV2/8 p5-2, C1m1 and C1m2 capsids. AAV vectors were prepared by triple transfection of Rep/cap plasmids (AAV 7m8, AAV2/8 p5-2, C1m1 and C1m2), AAV-CMV-GFP reporter plasmid (Addgene #67634) and pHelper plasmids using linear polyethylenimine (PEI) 25 kDa (Polysciences, US) and AAV293 (Cell Biolabs) cells as described in (Kimura et al., 2019). AAVs were purified for in vitro applications by Kimura et al., (2019) protocol. Cells were seeded in 12 well plates, 5×10⁴ cells/well in DMEM/F12 supplemented with 10% v/v foetal bovine serum (FBS) (Gibco) and 100 U/ml penicillin and 100 μg/ml streptomycin (Life Technologies) and transduced with AAVs at MOI: 1×10⁴-1×10⁵. Live cell imaging was performed at 48 hr and 72 hr using an EVOS Cell Imaging System (Thermo Fisher Scientific) or Olympus IX73 inverted microscope (Olympus). Nuclear staining was done using NucBlue™ Live ReadyProbes™ Reagent (Hoechst 33342) (Thermo Fisher). Shown are representative fluorescence microscopy images of ARPE19 cells transduced with the indicated AAV variants expressing GFP. Both C1m1 and C1m2 AAV2/8 capsids demonstrated GFP expression at 24 hr and 48 hr after transduction showing that they as efficient at transducing ARPE19 cells as “benchmarking” AAV2-7m8 variant and substantially more efficient than parental AAV2/8 p5-2 capsid (FIG. 7).

### Testing of Reporter Fluorescence of Various AAV-GFP Optimised Clones in Primary Retinal Cell Lines

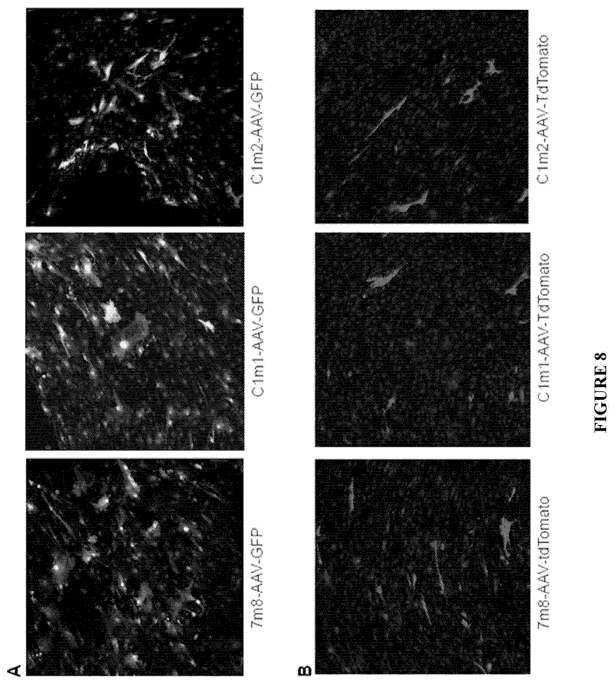

Human primary RPE mixed cultures (passage 4-7) were transduced in triplicate with AAVs containing tdTomato reporter plasmid (tdTomato cloned in pAAV-MCS, CellBiolabs), AAVs containing AAV-CMV-GFP reporter plasmid (Addgene #67634) and packaged with 7m8, AAV2/8 p5-2, C1m1 and C1m2 capsids. Cells were seeded in 1×10⁴ cells/well in DMEM/F12 supplemented with 10% v/v foetal bovine serum (FBS) (Gibco) and 100 U/ml penicillin and 100 μg/ml streptomycin (Life Technologies) and transduced with AAVs at MOI: 1×10⁴-1×10⁵. Cells were fixed with 4% PFA and imaged using IN Cell Analyzer 6500HS high-content imaging system. Nuclear staining was done using NucBlue™ Live ReadyProbes™ Reagent (Hoechst 33342) (Thermo Fisher). Shown are representative fluorescence microscopy images of human primary RPE cells transduced with the indicated AAV variants expressing GFP or TdTomato. Both C1m1 and C1m2 AAV2/8 capsids demonstrated GFP and tdTomato expression at 48 hr after transduction showing that they as efficient at transducing primary RPE cells as “benchmarking” AAV2-7m8 variant (FIG. 8A-B). Parental AAV2/8 p5-2 capsid showed negligible transduction of primary RPE cell cultures.

### Transduction Efficiency of Optimised AAV Vectors in Human Retinal Cell Lines and Explants

Firstly, the transduction efficiency was then tested in human APRE19 cells. ARPE19 cells were transduced in triplicate with AAVs containing secNanoLuc reporter and packaged with 7m8, AAV2/8 p5-2, C1m1 and C1m2 capsids. Secreted NanoLuc Luciferase (Promega) reporter gene was cloned into pAAV-MCS (Cell Biolabs) plasmid. The expression cassette was flanked by AAV2 inverted terminal repeats. SecNanoLuc reporter gene was expressed using the human cytomegalovirus (CMV) promoter. AAV vectors were prepared by triple transfection of Rep/cap plasmids (AAV 7m8, AAV2/8 p5-2, C1m1 and C1m2), secNanoLuc-pAAV-MCS and pHelper plasmids using linear polyethylenimine (PEI) 25 kDa (Polysciences, US) and AAV293 (Cell Biolabs) cells as described in (Kimura et al., 2019). AAVs were purified for in vitro applications by Kimura et al., (2019) protocol.

Cells were seeded in 24 well plates, 2×10⁴ cells/well in DMEM/F12 supplemented with 10% v/v foetal bovine serum (FBS) (Gibco) and 100 U/ml penicillin and 100 μg/ml streptomycin (Life Technologies) and transduced with AAVs at MOI 1×10⁴. Cells were transduced with AAVs in DMEM/F12 supplemented with 1% v/v FBS for 24 hrs. Following AAV transduction cells were cultured in 500 μL of DMEM/F12 (phenol red free) medium supplemented with 10% FBS (Gibco) and 100 U/ml penicillin and 100 μg/ml streptomycin (Life Technologies). 50 μL media aliquots were removed at the time points shown and frozen at −80° C. Aliquots were processed for measurement of secNanoLuc activity using Nano-Glo® Luciferase Assay system as recommended by manufacturer (Promega) and CLARIOstar Plus plate reader (BMG Labtech). Data for multiple time points were processed in Excel using transformation to calculate total luminescence at each time point (Promega). Total calculated RLU were analysed by GraphPad Prism 9 software. Three independent biological replicates were used.

Primary human RPE cells at passage 6-8 were seeded in 24 well plates, 5×10⁴ cells/well in DMEM/F12 supplemented with 10% v/v foetal bovine serum (FBS) (Gibco) and 100 U/ml penicillin and 100 μg/ml streptomycin (Life Technologies) and transduced with AAVs at MOI 1×10⁴. Cells were processed for secNanoLuc activity using Nano-Glo® Luciferase Assay system (Promega) as described in A).

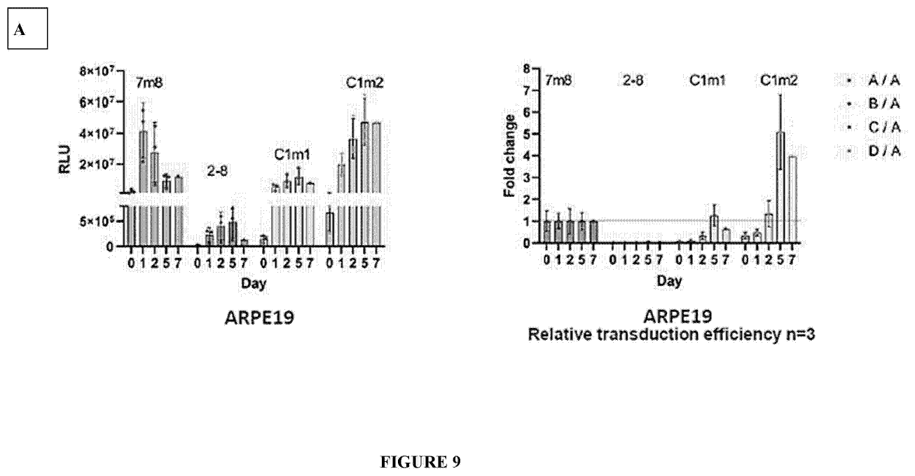

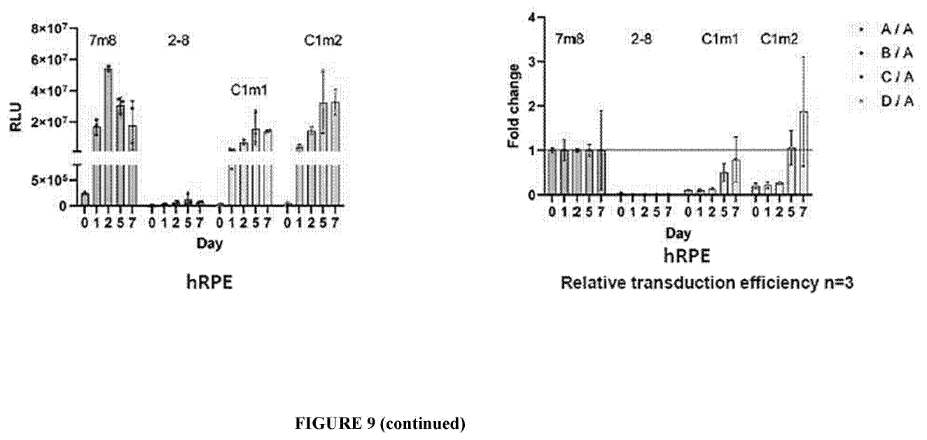

Both C1m1 and C1m2 AAV2/8 capsids demonstrated robust expression of secNanoLuc luciferase in ARPE19 and primary RPE cells. Accumulation of luciferase at day 5 and day 7 after transduction shows that they are more efficient at transducing human retinal pigment cells compared to AAV2-7m8 variant and parental AAAV2/8 p5-2 capsid. Relative transduction efficiency was especially high for C1m2 capsid on day 5 (up to 6-fold difference in comparison to AAV2-7m8 variant). These differences were observed during the whole duration of experiment (7 days). At day seven post-transduction, cells transduced using C1m2 AAV2/8 had significantly higher luciferase activity than AAV2-7m8 variant (FIGS. 9A and B). Therefore, peptide-inserts C1m1 and C1m2 at N590 of AAV8 capsid showed high transduction efficiency in human retinal cells.

The transduction efficiency was then tested in human retinal explants. Human post-mortem eye cups were dissected immediately upon receipt. After removal of the vitreous humour, punch biopsies were performed on the eye wall using disposable 3.5 mm (or 4 mm) biopsy punches (Livingstone International) to obtain retinal and retinal-pigment epithelium, choroid and sclera (RCS) explants. Up to 16 biopsies from each eye cup were obtained. Punch biopsies from the same eye cup were randomly grouped in 24 well plates and incubated for 24 hours in Dulbecco's Modified Eagle Medium: Nutrient Mixture F-12 (DMEM/F12) supplemented with 10% v/v foetal bovine serum (FBS) (Gibco) and 100 U/ml penicillin and 100 μg/ml streptomycin (Life Technologies) at 37° C. in 5% CO₂ humidified air atmosphere to recover from hypoxia and hypothermia before AAV transduction.

Retinal explants were transduced in triplicate with AAVs containing secNanoLuc reporter plasmid and packaged with 7m8, AAV2/8 p5-2, C1m1 and C1m2 capsids in DMEM/F12 supplemented with 1% v/v FBS for 48 hrs. secNanoLuc-AAVs were added at 2×10⁹ vg per explant. Secreted NanoLuc Luciferase (Promega) reporter gene was cloned into pAAV-MCS (Cell Biolabs) plasmid. The expression cassette was flanked by AAV2 inverted terminal repeats. SecNanoLuc reporter gene was expressed using the human cytomegalovirus (CMV) promoter. AAV vectors were prepared by triple transfection of Rep/cap plasmids (AAV 7m8, AAV2/8 p5-2, C1m1 and C1m2), secNanoLuc-pAAV-MCS and pHelper plasmids using linear polyethylenimine (PEI) 25 kDa (Polysciences, US) and AAV293 (Cell Biolabs) cells as described in (Kimura et al., 2019). AAVs were purified for in vitro applications by Kimura et al., (2019) protocol.

Following AAV transduction retinal explants were cultured in 24-well plates in 500 μL of DMEM/F12 (phenol red free) medium supplemented with 10% FBS (Gibco) and 100 U/ml penicillin and 100 μg/ml streptomycin (Life Technologies). 50 μL media aliquots were removed at the time points shown and frozen at −80° C. Aliquots were processed for measurement of secNanoLuc activity using Nano-Glo® Luciferase Assay system as recommended by manufacturer (Promega) and CLARIOstar Plus plate reader (BMG Labtech). Data for multiple time points were processed in Excel using transformation to calculate total luminescence at each time point (Promega). Total calculated RLU were analysed by GraphPad Prism 9 software.

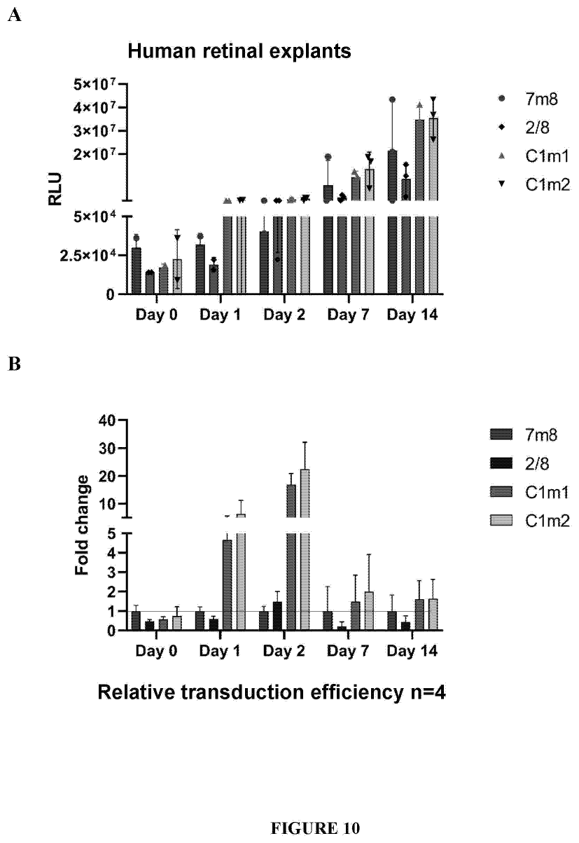

Both C1m1 and C1m2 AAV2/8 capsids demonstrated robust expression of secNanoLuc luciferase at day 1 and day2 after transduction showing that they are more efficient at transducing human retinal explants compared to AAV2-7m8 variant and parental AAAV2/8 p5-2 capsid (FIG. 10A). Relative transduction efficiency was especially high for C1m2 capsid on day 2 (up to 20-fold difference in comparison to AAV2-7m8 variant). These differences were continued during the whole duration of experiment (14 days). At day seven post-transduction, retinal explants transduced using C1m2 AAV2/8 had significantly higher luciferase activity than AAV2-7m8 variant (FIG. 10B). Therefore, peptide-inserts CIml and C1m2 at N590 of AAV8 capsid showed high transduction efficiency of human retinal explants.

The transduction efficiency was then tested in human epithelium, choroid and sclera (RCS) explants. Human post-mortem eye cups were dissected immediately upon receipt. After removal of the vitreous humour and retina, punch biopsies were performed on the eye wall using disposable 4 mm biopsy punches (Livingstone International) to obtain RCS explants. Up to 8 biopsies from each eye cup were obtained. Punch biopsies from the same eye cup were randomly grouped in 24 well plates and incubated for 24 hours in Dulbecco's Modified Eagle Medium: Nutrient Mixture F-12 (DMEM/F12) supplemented with 10% v/v foetal bovine serum (FBS) (Gibco) and 100 U/ml penicillin and 100 μg/ml streptomycin (Life Technologies) at 37° C. in 5% CO₂ humidified air atmosphere to recover from hypoxia and hypothermia before AAV transduction.

RCS explants were transduced in triplicate with AAVs containing secNanoLuc reporter plasmid and packaged with 7m8, AAV2/8 p5-2, C1m1 and C1m2 capsids. RCS explants were transduced with AAVs in DMEM/F12 supplemented with 1% v/v FBS for 48 hrs. secNanoLuc-AAVs were added at 2×10⁹ vg per explant.

Following AAV transduction retinal explants were cultured in 24-well plates in 500 μL of DMEM/F12 (phenol red free) medium supplemented with 10% FBS (Gibco) and 100 U/ml penicillin and 100 μg/ml streptomycin (Life Technologies). 50 μL media aliquots were removed at the time points shown and frozen at −80° C. Aliquots were processed for measurement of secNanoLuc activity using Nano-Glo® Luciferase Assay system as recommended by manufacturer (Promega) and CLARIOstar Plus plate reader (BMG Labtech). Data for multiple time points were processed in Excel using transformation to calculate total luminescence at each time point (Promega). Total calculated RLU were analysed by GraphPad Prism 9 software.

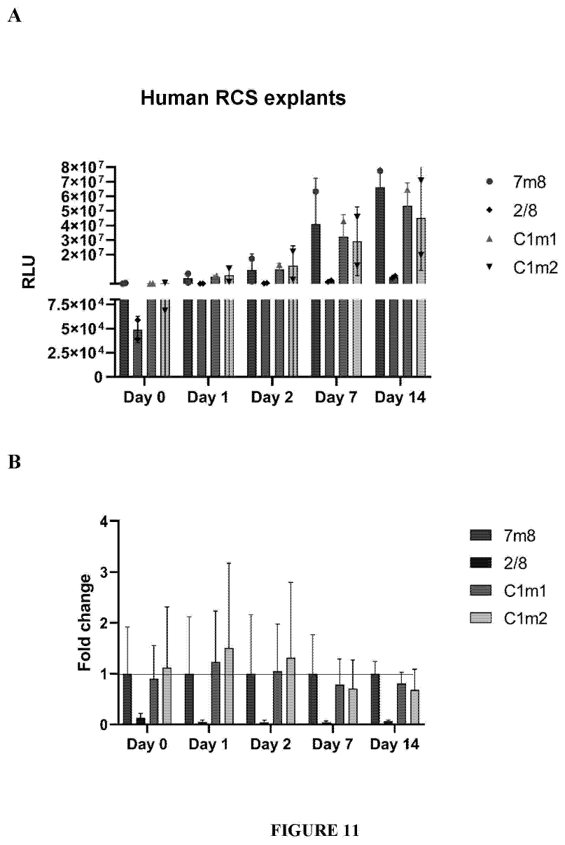

Both C1m1 and C1m2 AAV2/8 capsids demonstrated expression of secNanoLuc luciferase at day 1 and day2 after transduction showing that they are as efficient at transducing human RCS explants as “benchmarking” AAV2-7m8 variant and more efficient than parental AAAV2/8 p5-2 capsid (FIG. 11A-B).

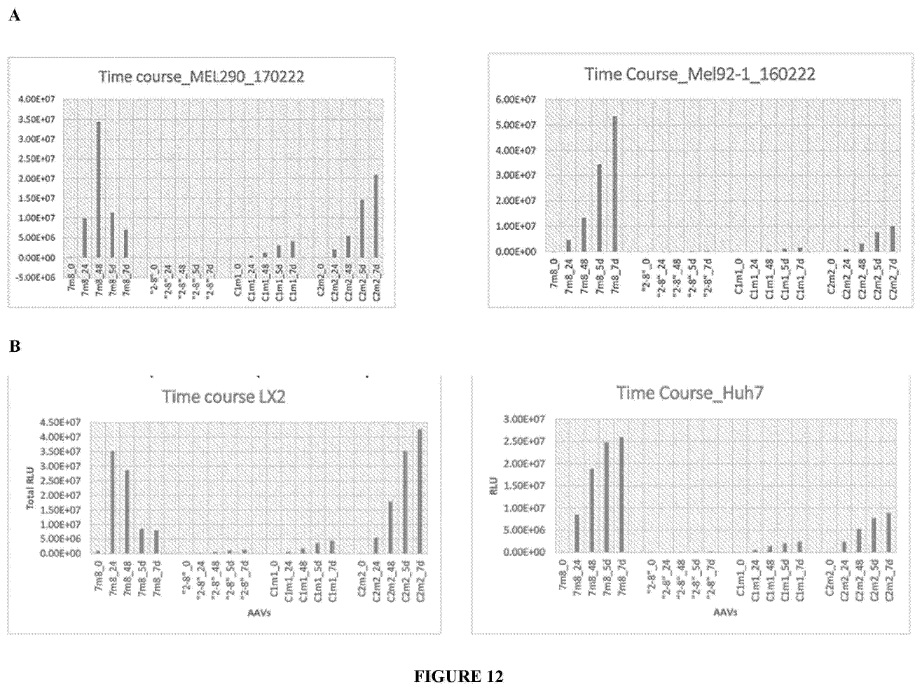

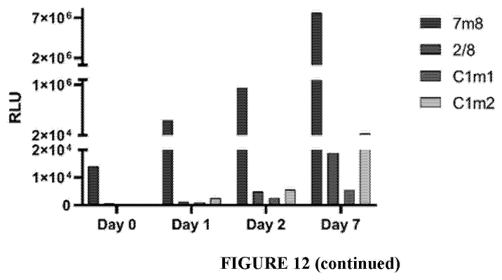

The inventors next sought to determine the effect of C1m1 and C1m2 AAV2/8 capsids in additional cell types. Ocular melanoma cells lines Mel290, 92-1 (FIG. 12A), LX2 Hepatic stellate cells and Huh 7 hepatocellular carcinoma cells (FIG. 12B) and transformed human umbilical vein endothelial cells (EA.hy926) (FIG. 12C) were transduced in triplicate with AAVs containing secNanoLuc reporter and packaged with 7m8, AAV2/8 p5-2, C1m1 and C1m2 capsids. Cells were seeded in 24 well plates, 2×10⁴ cells/well in DMEM/F12 supplemented with 10% v/v foetal bovine serum (FBS) (Gibco) and 100 U/ml penicillin and 100 μg/ml streptomycin (Life Technologies) and transduced with AAVs at MOI 1×10⁴. Cells were transduced with AAVs in DMEM/F12 supplemented with 1% v/v FBS for 24 hrs. Following AAV transduction cells were cultured in 500 μL of DMEM/F12 (phenol red free) medium supplemented with 10% FBS (Gibco) and 100 U/ml penicillin and 100 μg/ml streptomycin (Life Technologies). 50 μL media aliquots were removed at the time points shown and frozen at −80° C. Aliquots were processed for measurement of secNanoLuc activity using Nano-Glo® Luciferase Assay system as recommended by manufacturer (Promega) and CLARIOstar Plus plate reader (BMG Labtech). Data for multiple time points were processed in Excel using transformation to calculate total luminescence at each time point (Promega). Total calculated RLU were analysed by GraphPad Prism 9 software.

Both C1m1 and C1m2 AAV2/8 capsids demonstrated moderate tropism to ocular melanoma cells and hepatocellular carcinoma cells. Interestingly, C1m2 capsid showed increased tropism in LX2 hepatic stellate cells (SV40-transformed cell line), the major cell type responsible for liver fibrosis. The EA.hy926 cell line shows several features characteristic of vascular endothelial cells, and was efficiently transduced with 7m8 capsid. However, both C1m1 and C1m2 AAV2/8 capsids displayed lower tropism in the EA.hy926 cell line.

Novel AAV8 Variants with Modified Capsid Sequences C1m1 and C1m2 Transduce Rat Retinal Epithelium, Choroid and Sclera (RCS) Explants and Primary Rat RPE Cell Lines.

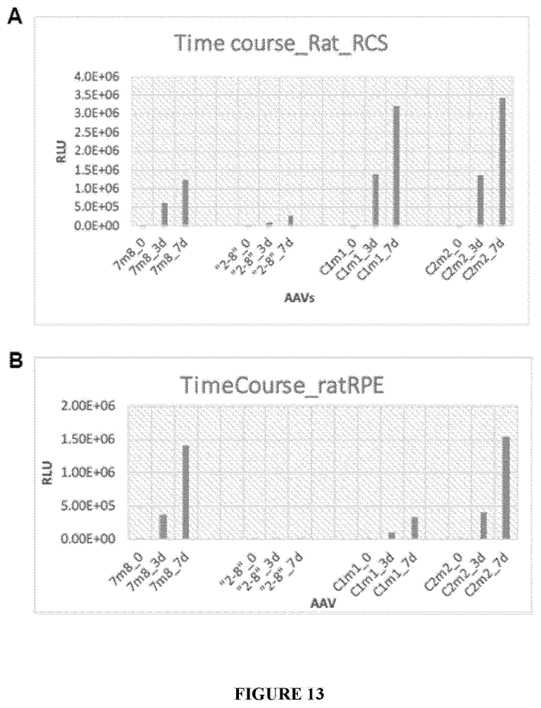

Rat (Wistar) eyes were dissected and placed into D-PBS containing antibiotics. After removal of the vitreous humour, punch biopsies were performed on the eye wall using disposable 2 mm biopsy punches (Livingstone International) to obtain retinal-pigment epithelium, choroid and sclera (RCS) explants. Up to 4 biopsies from each eye were obtained. Punch biopsies from the same rat eye were randomly grouped in 24 well plates and incubated for 24 hours in DMEM/F12 supplemented with 10% v/v foetal bovine serum (FBS) (Gibco) and 100 U/ml penicillin and 100 μg/ml streptomycin (Life Technologies) at 37° C. in 5% CO₂ before AAV transduction. Rat RCS explants were transduced in triplicate with AAVs containing secNanoLuc reporter and packaged with 7m8, AAV2/8 p5-2, C1m1 and C1m2 capsids. Explants were transduced with AAVs at 1×10⁹ vg/well in DMEM/F12 supplemented with 1% v/v FBS for 24 hrs. Following AAV transduction cells were cultured in 500 μL of DMEM/F12 (phenol red free) medium supplemented with 10% FBS (Gibco) and 100 U/ml penicillin and 100 μg/ml streptomycin (Life Technologies). 50 μL media aliquots were removed at the time points shown and frozen at −80° C. Aliquots were processed for measurement of secNanoLuc activity using Nano-Glo® Luciferase Assay system as recommended by manufacturer (Promega) and CLARIOstar Plus plate reader (BMG Labtech). Gain was adjusted to allow direct comparison of RLU between experiments with human and rat RCS explants. Data for multiple time points were processed in Excel using transformation to calculate total luminescence at each time point (Promega). As shown in FIG. 13A, transduction of RCS explants with AAVs containing C1m1 and C2m2 resulted in significant increases to transduction efficiency when compared to AAV 7m8 over 3- and 7-days treatment.

Primary rat RPE mixed cell cultures were also transduced in triplicate with AAVs containing secNanoLuc reporter and packaged with 7m8, AAV2/8 p5-2, C1m1 and C1m2 capsids. Cells were seeded in 24 well plates, 5×10⁴ cells/well in DMEM/F12 supplemented with 10% v/v foetal bovine 20 serum (FBS) (Gibco) and 100 U/ml penicillin and 100 μg/ml streptomycin (Life Technologies) and transduced with AAVs at MOI 1×10⁴. Cells were transduced with AAVs in DMEM/F12 supplemented with 1% v/v FBS for 24 hrs. Following AAV transduction cells were cultured in 500 μL of DMEM/F12 (phenol red free) medium supplemented with 10% FBS (Gibco) and 100 U/ml penicillin and 100 μg/ml streptomycin (Life Technologies). 50 μL media aliquots were removed at the time points shown and frozen at −80° C. Aliquots were processed for measurement of secNanoLuc activity using Nano-Glo® Luciferase Assay system as recommended by manufacturer (Promega) and CLARIOstar Plus plate reader (BMG Labtech). Gain was adjusted to allow direct comparison of RLU between experiments with human and rat cell cultures. Data for multiple time points were processed in Excel using transformation to calculate total luminescence at each time point (Promega). As shown in FIG. 13B, transduction of rat RPE cells with AAVs containing C1m1 and C2m2 resulted in increases to transduction efficiency over 3 and 7 days treatment.

Transduction Assays with Potential Therapeutic Gene IL10 in Primary RPE Cell Lines Using Modified Capsids C1m1 and C1m2.

Primary human RPE cells at passage 6-8 were transduced in triplicate with AAVs containing IL 10 gene and packaged with 7m8, AAV2/8 p5-2, C1m1 and C1m2 capsids. IL10 gene was expressed using the human cytomegalovirus (CMV) promoter. AAV vectors were prepared by triple transfection of Rep/cap plasmids (AAV 7m8, AAV2/8 p5-2, C1m1 and C1m2), IL-10-pAAV and pHelper plasmids into AAV293 cell line (Cell Biolabs). AAV-CMV-TdTomato was used as a non-specific control.

Cells were seeded in 24 well plates, 5×10⁴ cells/well in DMEM/F12 supplemented with 10% v/v foetal bovine serum (FBS) (Gibco) and 100 U/ml penicillin and 100 μg/ml streptomycin (Life Technologies) and transduced with AAVs at MOI 1×10⁴. Cells were transduced with AAVs in DMEM/F12 supplemented with 1% v/v FBS for 24 hrs. Following AAV transduction cells were cultured in 500 μL of DMEM/F12 (phenol red free) medium supplemented with 10% FBS (Gibco) and 100 U/ml penicillin and 100 μg/ml streptomycin (Life Technologies). 100 μL media aliquots were removed at the time points shown and frozen at −80° C. Aliquots were processed for measurement of IL 10 protein using IL10 ELISA kit as recommended by manufacturer (Thermo Fisher Scientific). Data for multiple time points were analysed by GraphPad Prism 9 software.

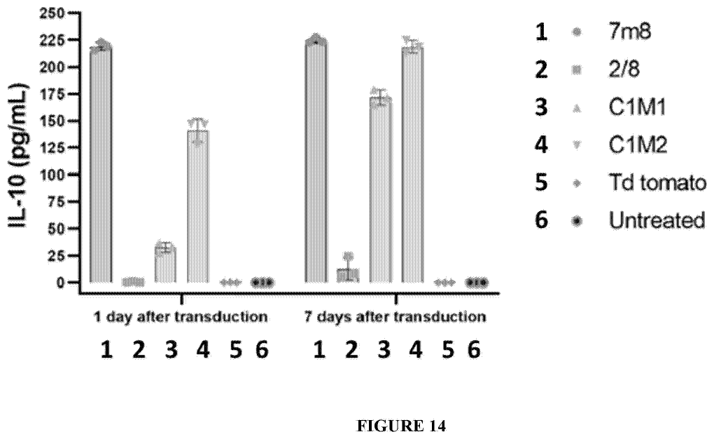

Both C1m1 and C1m2 AAV2/8 capsids demonstrated expression of IL 10 gene in primary RPE cells. Substantial amount of IL10 protein at day 7 after transduction showed that modified capsids are more efficient at transducing human retinal pigment cells compared to parental AAV2/8 p5-2 capsid. IL10 expression was especially high for C1m2 capsid on day 7 and was comparable to AAV2-7m8 variant. At day seven post-transduction, cells transduced using C1m2 AAV2/8 had significantly higher IL 10 expression than AAV2/8 p5-2 capsid and C1m1 capsid (FIG. 14). Therefore, the data show that insertion of C1m1 or C1m2 at position N590 of AAV8 capsid provide for efficient delivery of IL10 as a therapeutic gene into human retinal cells.

Novel AAV8 Variants with Modified Capsid Sequences C1m1 and C1m2 Expressing Endostatin-Angiostatin Fusion Protein Regulate Choroidal Sprouting in Ex Vivo Human Explants.

Human post-mortem eye cups were dissected immediately upon receipt. After removal of the vitreous humour, punch biopsies were performed using disposable 2 mm biopsy punches (Livingstone International) to obtain retinal-pigment epithelium, choroid and sclera (RCS) explants. Up to 12 biopsies from each eye cup were obtained. Punch biopsies from the same eye cup were randomly grouped in 24 well plates and incubated for 24 hours in Dulbecco's Modified Eagle Medium: Nutrient Mixture F-12 (DMEM/F12) supplemented with 10% v/v foetal bovine serum (FBS) (Gibco) and 100 U/ml penicillin and 100 μg/ml streptomycin (Life Technologies) at 37° C. in 5% CO₂ to recover from hypoxia and hypothermia before AAV transduction.

Retinal explants were transduced in triplicate with AAVs containing a secretable Endostatin-Angiostatin fusion gene and packaged with C1m1 and C1m2 capsids in DMEM/F12 supplemented with 1% v/v FBS for 48 hrs. Endostatin-Angiostatin AAVs were added at 2×10⁹ vg per explant. Secreted Endostatin-Angiostatin fusion gene was cloned into pAAV-MCS (Cell Biolabs). Secreted Endostatin-Angiostatin fusion gene was expressed using the human cytomegalovirus (CMV) promoter. Control explants were left untreated or treated with non-specific AAV containing fluorescent reporter gene.

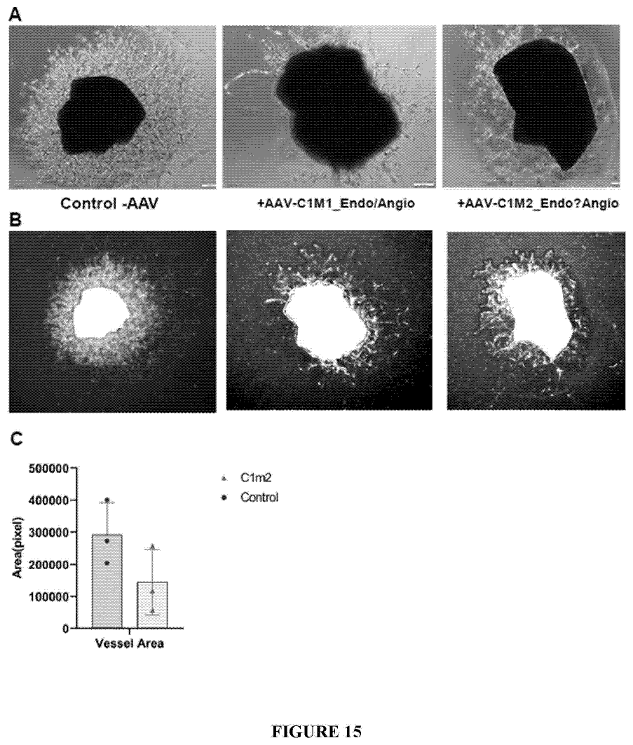

Human choroidal sprouting assay ex vivo was adapted from a protocol for mouse eye explants (Shao et al., 2013). After transduction, the choroid explants were embedded in 30 μL cold growth factor reduced Geltrex (Thermo Fisher) in 24-well glass bottom plates for high resolution imaging (Cellvis). Geltrex “domes” were allowed to polymerize for 20 min at +37° C. in TC incubator. The explants were grown in EBM-2 media (Lonza) supplemented with 2.5% FBS, growth factors, including VEGF165, ascorbic acid and penicillin-streptomycin at 37° C. with 5% CO2. The choroidal sprouts originating from explants were photographed on day 7 as shown in FIG. 15A. Individual explants were photographed using phase contrast optics on Olympus IX73 microscope at 4× magnification. The representative images of choroidal sprouting are shown. The sprouting area was quantified using FIJI/Image J (Schindelin et al., 2012) (FIG. 15B). The quantification results of sprouting area after transduction with AAV-C1m2 expressing Endostatin-Angiostatin fusion gene are shown (n=3 biological replicates) (FIG. 15C). As expected, choroidal sprouts originating from explants were significantly inhibited in explants transduced with AAVs containing a secretable Endostatin-Angiostatin fusion gene and packaged with C1m1 and C1m2 capsids.

### REFERENCES

1 Wang, D., et al., Adeno-associated virus vector as a platform for gene therapy delivery. Nat. Rev. Drug Discov. 18, 358-378 (2019).
2. Boutin, S. et al. Prevalence of serum IgG and neutralizing factors against adeno-associated virus (AAV) types 1, 2, 5, 6, 8, and 9 in the healthy population: implications for gene therapy using AAV vectors. Hum. Gene Ther. 21, 704-712 (2010).
3. Calcedo, R., Vandenberghe, L. H., Gao, G., Lin, J. & Wilson, J. M. Worldwide epidemiology of neutralizing antibodies to adeno-associated viruses. J. Infect. Dis. 199, 381-390 (2009).
4. Dong, J.-Y., Fan, P.-D. & Frizzell, R. A. Quantitative Analysis of the Packaging Capacity of Recombinant Adeno-Associated Virus. Hum. Gene Ther. 7, 2101-2112 (1996).
5. Agbandje-McKenna, M. & Kleinschmidt, J. AAV capsid structure and cell interactions. Methods Mol. Biol. Clifton NJ. 807, 47-92 (2011).
6. Samulski, R. J. & Muzyczka, N. AAV-Mediated Gene Therapy for Research and Therapeutic Purposes. Annu. Rev. Virol. 1, 427-451 (2014).
7. Zhong, L. et al., Next generation of adeno-associated virus 2 vectors: point mutations in tyrosines lead to high-efficiency transduction at lower doses. Proc. Natl. Acad. Sci. U.S.A 105, 7827-7832 (2008).
8. Aslanidi, G. V. et al., High-efficiency transduction of human monocyte-derived dendritic cells by capsid-modified recombinant AAV2 vectors. Vaccine 30, 3908-3917 (2012).
9. Aslanidi, G. V. et al., Optimization of the capsid of recombinant adeno-associated virus 2 (AAV2) vectors: the final threshold? PloS One 8, e59142 (2013).
10. Li, B. et al. Site-Directed Mutagenesis of Surface-Exposed Lysine Residues Leads to Improved Transduction by AAV2, But Not AAV8, Vectors in Murine Hepatocytes In Vivo. Hum. Gene Ther. Methods 26, 211-220 (2015).
11. Shi, W., Arnold, G. S. & Bartlett, J. S. Insertional mutagenesis of the adeno-associated virus type 2 (AAV2) capsid gene and generation of AAV2 vectors targeted to alternative cell-surface receptors. Hum. Gene Ther. 12, 1697-1711 (2001).
12. Xie, Q. et al., The atomic structure of adeno-associated virus (AAV-2), a vector for human gene therapy. Proc. Natl. Acad. Sci. 99, 10405-10410 (2002).
13. Yang, L. et al., A myocardium tropic adeno-associated virus (AAV) evolved by DNA shuffling and in vivo selection. Proc. Natl. Acad. Sci. U.S.A 106, 3946-3951 (2009).
14. Bartel, M. A., Weinstein, J. R. & Schaffer, D. V. Directed evolution of novel adeno-associated viruses for therapeutic gene delivery. Gene Ther. 19, 694-700 (2012).
15. Zinn, E. et al., In Silico Reconstruction of the Viral Evolutionary Lineage Yields a Potent Gene Therapy Vector. Cell Rep. 12, 1056-1068 (2015).
16. Bordet, T. & Behar-Cohen, F. Ocular gene therapies in clinical practice: viral vectors and nonviral alternatives. Drug Discov. Today 24, 1685-1693 (2019).
17. Ramlogan-Steel, C. A. et al., Gene therapy and the adeno-associated virus in the treatment of genetic and acquired ophthalmic diseases in humans: Trials, future directions and safety considerations. Clin. Experiment. Ophthalmol. 47, 521-536 (2019).
18. Wiley, L. A. et al., Assessment of Adeno-Associated Virus Serotype Tropism in Human Retinal Explants. Hum. Gene Ther. 29, 424-436 (2018).
19. Dalkara, D. et al., In Vivo-Directed Evolution of a New Adeno-Associated Virus for Therapeutic Outer Retinal Gene Delivery from the Vitreous. Sci. Transl. Med. 5, 189ra76-189ra76 (2013).
20. Halbert, C. L. et al., Prevalence of Neutralizing Antibodies against Adeno-Associated Virus (AAV) Types 2, 5, and 6 in Cystic Fibrosis and Normal Populations: Implications for Gene Therapy using AAV Vectors. Hum. Gene Ther. 17, 440-447 (2006).
21. Li, J., Samulski, R. J. & Xiao, X. Role for highly regulated rep gene expression in adeno-associated virus vector production. J. Virol. 71, 5236-5243 (1997).
22. Petrs-Silva, H. et al., High-efficiency Transduction of the Mouse Retina by Tyrosine-mutant AAV Serotype Vectors. Mol. Ther. 17, 463-471 (2009).
23. Kay, C. N. et al., Targeting Photoreceptors via Intravitreal Delivery Using Novel, Capsid-Mutated AAV Vectors. PLOS ONE 8, e62097 (2013).
24. Nicklin, S. A. et al., Efficient and Selective AAV2-Mediated Gene Transfer Directed to Human Vascular Endothelial Cells. Mol. Ther. 4, 174-181 (2001).
25. Grifman, M. et al., Incorporation of Tumor-Targeting Peptides into Recombinant Adeno-associated Virus Capsids. Mol. Ther. 3, 964-975 (2001).
26. Girod, A. et al., Genetic capsid modifications allow efficient re-targeting of adeno-associated virus type 2. Nat. Med. 5, 1052-1056 (1999).
27. Müller, O. J. et al. Random peptide libraries displayed on adeno-associated virus to select for targeted gene therapy vectors. Nat. Biotechnol. 21, 1040-1046 (2003).
28. Kimura T. et al., Production of adeno-associated virus vectors for in vitro and in vivo applications. Sci Rep. 9 (1): 13601 (2019).
29. Brown N. P. et al., MView: a web-compatible database search or multiple alignment viewer. Bioinformatics. 14 (4): 380-1 (1998).
29. Khabou H. et al., Insight into the mechanisms of enhanced retinal transduction by the engineered AAV2 capsid variant-7m8. Biotechnol Bioeng. December; 113 (12): 2712-2724 (2016).
30. Büning, H., & Srivastava, A. Capsid Modifications for Targeting and Improving the Efficacy of AAV Vectors. Molecular therapy. Methods & Clinical Development, 12, 248-265 (2019).
31. Pavlou M et al., Novel AAV capsids for intravitreal gene therapy of photoreceptor disorders. EMBO Mol Med., 13 (4) (2021).
32. Börner, K. et al., Pre-arrayed Pan-AAV Peptide Display Libraries for Rapid Single-Round Screening. Molecular therapy: the journal of the American Society of Gene Therapy, 28 (4), 1016-1032 (2020).
33. Raupp, C. et al., The Threefold Protrusions of Adeno-Associated Virus Type 8 Are Involved in Cell Surface Targeting as Well as Postattachment Processing. J Virol., 86 (17): 9396-9408 (2012).
34. Nam et al., Structure of Adeno-Associated Virus Serotype 8, a Gene Therapy Vector, J Virol., v.81 (22) (2007).
35. Mietzsch, M. et al., Completion of the AAV Structural Atlas: Serotype Capsid Structures Reveals Clade-Specific Features, Viruses, 13 (1): 101 (2021).
36. Shao, Z., et al., Choroid sprouting assay: an ex vivo model of microvascular angiogenesis. PLOS One. July 26; 8 (7): e69552 (2013).
37. Schindelin, J., et al., Fiji: an open-source platform for biological-image analysis. Nature Methods, 9 (7), 676-682 (2012).
38. Gao, G., et al., Novel adeno-associated viruses from rhesus monkeys as vectors for human gene therapy. PNAS, 99 (18) 11854-11859 (2002).
39. Wu Z., et al., Single amino acid changes can influence titer, heparin binding, and tissue tropism in different adeno-associated virus serotypes. J Virol., 80 (22) 11393-7 (2006).
40. Perbal, J., A Practical Guide to Molecular Cloning, John Wiley and Sons (1984).
41. Sambrook, J., et al., Molecular Cloning: A Laboratory Manual, Cold Spring Harbour Laboratory Press (1989).
42. Brown T. A., (editor) Essential Molecular Biology: A Practical Approach. Volumes 1 and 2, IRL Press (1991).
43. Glover D. M., and Hames B. D., (editors), DNA Cloning: A Practical Approach. Volumes 1-4, IRL Press (1995 and 1996)
44. Ausubel F. M., et al., (editors), Current Protocols in Molecular Biology. Greene Pub. Associates and Wiley-Interscience (1988, including all updates until present),
45. Coligan J. E., et al. (editors) Current Protocols in Immunology. John Wiley & Sons (including all updates until present).
46. Nei M. and Kumar S., Molecular Evolution and Phylogenetics. Oxford University Press, New York (2000).
47. Needleman S. B., and Wunsch C. D., A general method applicable to the search for similarities in the amino acid sequence of two proteins. J Mol Biol 48 (3): 443-53 (1970).
48. Knipe D. M., and Howley P. M., Clinical Infectious Dsieases, Field's Virology. Lippincott Williams & Wilkins, Philadelphia, ed. Sixth (2013).
49. Vasileva & Jessberger, Precise hit: adeno-associated virus in gene targeting, Nature reviews. Microbiology, 3:837-847 (2005).
50. Muzyczka, Use of adeno-associated virus as a general transduction vector for mammalian cells, Current Topics in Microbiol, and Immunol. 158:97-129 (1992).
51. Kotin, Prospects for the use of adeno-associated virus as a vector for human gene therapy, Human Gene Therapy 5:793-801 (1994).
52. Zhou et al., Adeno-associated virus 2-mediated high efficiency gene transfer into immature and mature subsets of hematopoietic progenitor cells in human umbilical cord blood, J Exp. Med. 179:1867-1875 (1994).
53. Zolotukhin et al., Production and purification of serotype 1, 2, and 5 recombinant adeno-associated viral vectors. Methods 28:158-167 (2002).
54. Samulski et al., A recombinant plasmid from which an infectious adeno-associated virus genome can be excised in vitro and its use to study viral replication, J Virology, 6:3096-3101 (1987).
55. Clement et al., Large-scale adeno-associated viral vector production using a herpesvirus-based system enables manufacturing for clinical studies, Hum Gene Ther., 20 (8): 796-806 (2009).
56. Duan et al., Enhancement of muscle gene delivery with pseudotyped adeno-associated virus type 5 correlates with myoblast differentiation, J. Virol., 75:7662-7671 (2001).
57. Halbert et al., Repeat transduction in the mouse lung by using adeno-associated virus vectors with different serotypes, J. Virol., 74:1524-1532 (2000).
58. Zolotukhin et al., Production and purification of serotype 1, 2, and 5 recombinant adeno-associated viral vectors, Methods, 28:158-167 (2002).
59. Auricchio et al., Exchange of surface proteins impacts on viral vector cellular specificity and transduction characteristics: the retina as a model, Hum. Mol. Genet., 10:3075-3081 (2001).
60. Kozomara et al., miRBase: annotating high confidence microRNAs using deep sequencing data Nuc Acids Res, 42:68-73 (2013).
61. Griffiths-Jones, S., The microRNA Registry, Nuc Acids Res, 1:32 (2004).
62. Remington's Pharmaceutical Sciences (Mack Publishing Co. N.J. USA (1991).
63. Remington: The Science and Practice of Pharmacy, 22nd edition. Pharmaceutical Press, (2012).

1. A recombinant adeno-associated virus (AAV) capsid protein comprising an insertion according to the sequence set forth in SEQ ID NO:1 or functional equivalents thereof.

2. The recombinant AAV capsid protein according to claim 1, wherein the insertion is in the VR-VIII of an AAV8 capsid protein or in a corresponding position of a capsid protein of an AAV serotype other than AAV8, wherein the insertion is relative to a parental AAV capsid protein.

3. The recombinant AAV capsid protein according to claim 1, wherein the insertion is located at a position corresponding to amino acid 590, preferably of the capsid protein of AAV8 or in a corresponding position of a capsid protein of an AAV serotype other than AAV8.

4. The recombinant AAV capsid protein according to claim 1, wherein the insertion is relative to a parental AAV capsid protein according to the sequence set forth in SEQ ID NO:121 or 125.

5. The recombinant AAV capsid protein according to claim 1, wherein the insertion comprises a sequence according to any one of SEQ ID NO:2-108.

6. The recombinant AAV capsid protein according to claim 1, wherein the insertion comprises a sequence that differs by no more than 1 or 2 amino acids when compared with the sequence set forth in SEQ ID NO:1.

7. The recombinant AAV capsid protein according to claim 1, wherein the insertion comprises a sequence set forth in SEQ ID NO: 109 or SEQ ID NO:110.

8. The recombinant AAV capsid protein according to claim 1, wherein
(i) the insertion comprises a linker flanking the N-terminal and/or C-terminal end of the insertion, optionally, comprising a sequence set forth in SEQ ID NO:111 and/or SEQ ID NO: 112,
further optionally wherein the insertion comprises a sequence set forth in SEQ ID NO: 129 or 138; and/or
(ii) the insertion is in one or more or all of the VP1, VP2 and VP3 capsid proteins, preferably the VP3 capsid protein, optionally comprising a sequence set forth in SEQ ID NOs: 130-133 or 139-140; and/or
(iii) the recombinant capsid protein comprises a mutation selected from one or more or all of Y447F, T494V and Y733F, preferably comprising each of Y447F, T494V and Y733F in the AAV8 capsid protein, optionally wherein the recombinant AAV capsid protein comprises a sequence set forth in SEQ ID NO: 135 or SEQ ID NO: 142; and/or
(iv) the AAV capsid protein is an AAV2, AAV4, AAV7 or AAV10 capsid protein.

9-13. (canceled)

14. A recombinant adeno-associated virus (AAV) particle comprising the AAV capsid protein according to claim 1.

15. An isolated nucleic acid encoding a recombinant adeno-associated virus (AAV) capsid protein according to claim 1.

16. The isolated nucleic acid according to claim 15, comprising a sequence set forth in SEQ ID NO:134 or 141.

17-32. (canceled)

33. A pharmaceutical composition comprising an AAV particle according to claim 14 and one or more carriers or excipients.

34. An ex vivo, in vitro or in vivo method for increasing transduction efficiency of an adeno-associated virus (AAV) particle within a target cell, tissue or organ, the method comprising contacting the target cell, tissue or organ with an AAV particle according to claim 14 under conditions sufficient for transduction of the AAV particle within the target cell, tissue or organ, wherein transduction efficiency is increased when compared to an AAV particle not having the capsid insertion.

35-37. (canceled)

38. A method for reducing an immune response to an adeno-associated virus (AAV) particle in a target cell, tissue or organ, the method comprising contacting the target cell, tissue or organ with an AAV particle according to claim 14 under conditions sufficient for transduction of the AAV particle in the target cell, tissue or organ, wherein the immune response is reduced when compared to an AAV particle not having the capsid insertion.

39. The method according to claim 34, wherein the target cell is a retinal cell, the target tissue is retinal tissue and the target organ is the eye.

40. A method for treating a condition, disorder or disease in a subject in need thereof, comprising administering an AAV particle according to claim 14, thereby treating the condition, disorder or disease in the subject.

41-42. (canceled)

43. The method according to claim 40, wherein
(i) the condition, disorder or disease is an ocular disease, optionally selected from the group consisting of retinitis pigmentosa, diabetic retinopathy, cystoid macular oedema, clinically significant macular oedema, uveitis, iritis, giant cell arteritis, vasculitis, pars planitis, corneal transplant rejection, intraocular inflammation or lamellar corneal transplant rejection, macular degeneration, central retinal vein occlusion, branch retinal vein occlusion and ocular neovascularisation; and/or
(ii) the subject has at least one symptom of an ocular disorder selected from the group comprising decreased peripheral vision, decreased central vision, decreased night vision and loss of colour perception; and/or
(iii) the treating comprises administration of an immunosuppressant, preferably prior to administration of the recombinant AAV or composition thereof, optionally wherein the immunosuppressant is administered intravenously, orally, subcutaneously or intramuscularly; and/or
(iv) the treating comprises administration of an additional therapy, optionally selected from the group comprising surgery, lens replacement with an intraocular lens, laser surgery or medication; and/or
(v) the recombinant AAV particle or composition is administered intravitreally.

44. (canceled)

45. The method according to claim 40, wherein the administered AAV particle or composition comprising comprises:
a) a kill switch comprising a first site-specific recombination sequence and a second site-specific recombination sequence;
b) a regulatable element operably linked to a nucleic acid sequence encoding a therapeutic molecule, wherein activity of the regulatable element is regulated by a regulator compound; and
c) a constitutive promoter operably linked to a nucleic acid sequence encoding a regulator compound-binding polypeptide which is capable of binding a regulator compound, wherein upon binding the regulator compound, the regulator compound-binding polypeptide regulates expression of the therapeutic molecule, wherein activation of the kill switch by recombination between the first site-specific recombination sequence and the second site-specific recombination sequence silences expression of the nucleic acid encoding the therapeutic molecule from the cassette.

46. The method according to claim 45, wherein
(i) the AAV particle is administered intravitreally or subretinally and the kill switch is activated by administering a site-specific recombinase or a nucleic acid encoding a site-specific recombinase, wherein the site-specific recombinase catalyses the recombination between a first site-specific recombination sequence and a second site-specific recombination sequence, thereby silencing expression of the therapeutic molecule; and/or
(ii) the treatment comprises administering the regulator compound to the eye topically or in eye drops.

47-51. (canceled)

52. An isolated mammalian cell comprising a recombinant adeno-associated virus (AAV) particle according to claim 14, optionally wherein the mammalian cell is a human cell, preferably a human retinal cell, optionally selected from the group consisting of photoreceptors, retinal ganglion cells, bipolar cells, trabecular meshwork, retinal pigment epithelium cells, amacrine cells, astrocytes, horizontal cells, microglia or Muller glia cells.

53-56. (canceled)


SEQUENCE LISTING (143 sequences)

SEQ ID NO: 1 - protein, 5 aa, synthetic construct
GNRVD

SEQ ID NO: 2 - protein, 5 aa, synthetic construct
PNRVD

SEQ ID NO: 3 - protein, 5 aa, synthetic construct
ANRVD

SEQ ID NO: 4 - protein, 5 aa, synthetic construct
GQRVD

SEQ ID NO: 5 - protein, 5 aa, synthetic construct
GHRVD

SEQ ID NO: 6 - protein, 5 aa, synthetic construct
PQRVD

SEQ ID NO: 7 - protein, 5 aa, synthetic construct
PHRVD

SEQ ID NO: 8 - protein, 5 aa, synthetic construct
AQRVD

SEQ ID NO: 9 - protein, 5 aa, synthetic construct
AHRVD

SEQ ID NO: 10 - protein, 5 aa, synthetic construct
GNKVD

SEQ ID NO: 11 - protein, 5 aa, synthetic construct
PNKVD

SEQ ID NO: 12 - protein, 5 aa, synthetic construct
ANKVD

SEQ ID NO: 13 - protein, 5 aa, synthetic construct
GQKVD

SEQ ID NO: 14 - protein, 5 aa, synthetic construct
GHKVD

SEQ ID NO: 15 - protein, 5 aa, synthetic construct
PQKVD

SEQ ID NO: 16 - protein, 5 aa, synthetic construct
PHKVD

SEQ ID NO: 17 - protein, 5 aa, synthetic construct
AQKVD

SEQ ID NO: 18 - protein, 5 aa, synthetic construct
AHKVD

SEQ ID NO: 19 - protein, 5 aa, synthetic construct
GNRID

SEQ ID NO: 20 - protein, 5 aa, synthetic construct
AQRAD

SEQ ID NO: 21 - protein, 5 aa, synthetic construct
AHRID

SEQ ID NO: 22 - protein, 5 aa, synthetic construct
AHRLD

SEQ ID NO: 23 - protein, 5 aa, synthetic construct
AHRMD

SEQ ID NO: 24 - protein, 5 aa, synthetic construct
AHRFD

SEQ ID NO: 25 - protein, 5 aa, synthetic construct
AHRAD

SEQ ID NO: 26 - protein, 5 aa, synthetic construct
GNKID

SEQ ID NO: 27 - protein, 5 aa, synthetic construct
GNKLD

SEQ ID NO: 28 - protein, 5 aa, synthetic construct
GNKMD

SEQ ID NO: 29 - protein, 5 aa, synthetic construct
GNKFD

SEQ ID NO: 30 - protein, 5 aa, synthetic construct
GNKAD

SEQ ID NO: 31 - protein, 5 aa, synthetic construct
PNKID

SEQ ID NO: 32 - protein, 5 aa, synthetic construct
PNKLD

SEQ ID NO: 33 - protein, 5 aa, synthetic construct
PNKMD

SEQ ID NO: 34 - protein, 5 aa, synthetic construct
PNKFD

SEQ ID NO: 35 - protein, 5 aa, synthetic construct
PNKAD

SEQ ID NO: 36 - protein, 5 aa, synthetic construct
ANKID

SEQ ID NO: 37 - protein, 5 aa, synthetic construct
ANKLD

SEQ ID NO: 38 - protein, 5 aa, synthetic construct
ANKMD

SEQ ID NO: 39 - protein, 5 aa, synthetic construct
GNRLD

SEQ ID NO: 40 - protein, 5 aa, synthetic construct
GNRMD

SEQ ID NO: 41 - protein, 5 aa, synthetic construct
GNRFD

SEQ ID NO: 42 - protein, 5 aa, synthetic construct
GNRAD

SEQ ID NO: 43 - protein, 5 aa, synthetic construct
PNRID

SEQ ID NO: 44 - protein, 5 aa, synthetic construct
PNRLD

SEQ ID NO: 45 - protein, 5 aa, synthetic construct
PNRMD

SEQ ID NO: 46 - protein, 5 aa, synthetic construct
PNRFD

SEQ ID NO: 47 - protein, 5 aa, synthetic construct
PNRAD

SEQ ID NO: 48 - protein, 5 aa, synthetic construct
ANRID

SEQ ID NO: 49 - protein, 5 aa, synthetic construct
ANRLD

SEQ ID NO: 50 - protein, 5 aa, synthetic construct
ANRMD

SEQ ID NO: 51 - protein, 5 aa, synthetic construct
ANRFD

SEQ ID NO: 52 - protein, 5 aa, synthetic construct
ANRAD

SEQ ID NO: 53 - protein, 5 aa, synthetic construct
GQRID

SEQ ID NO: 54 - protein, 5 aa, synthetic construct
GQRLD

SEQ ID NO: 55 - protein, 5 aa, synthetic construct
GQRMD

SEQ ID NO: 56 - protein, 5 aa, synthetic construct
GQRFD

SEQ ID NO: 57 - protein, 5 aa, synthetic construct
GQRAD

SEQ ID NO: 58 - protein, 5 aa, synthetic construct
ANKFD

SEQ ID NO: 59 - protein, 5 aa, synthetic construct
ANKAD

SEQ ID NO: 60 - protein, 5 aa, synthetic construct
GQKID

SEQ ID NO: 61 - protein, 5 aa, synthetic construct
GQKLD

SEQ ID NO: 62 - protein, 5 aa, synthetic construct
GQKMD

SEQ ID NO: 63 - protein, 5 aa, synthetic construct
GQKFD

SEQ ID NO: 64 - protein, 5 aa, synthetic construct
GQKAD

SEQ ID NO: 65 - protein, 5 aa, synthetic construct
GHKID

SEQ ID NO: 66 - protein, 5 aa, synthetic construct
GHKLD

SEQ ID NO: 67 - protein, 5 aa, synthetic construct
GHKMD

SEQ ID NO: 68 - protein, 5 aa, synthetic construct
GHKFD

SEQ ID NO: 69 - protein, 5 aa, synthetic construct
GHKAD

SEQ ID NO: 70 - protein, 5 aa, synthetic construct
PQKID

SEQ ID NO: 71 - protein, 5 aa, synthetic construct
PQKLD

SEQ ID NO: 72 - protein, 5 aa, synthetic construct
PQKMD

SEQ ID NO: 73 - protein, 5 aa, synthetic construct
PQKFD

SEQ ID NO: 74 - protein, 5 aa, synthetic construct
PQKAD

SEQ ID NO: 75 - protein, 5 aa, synthetic construct
PHKID

SEQ ID NO: 76 - protein, 5 aa, synthetic construct
PHKLD

SEQ ID NO: 77 - protein, 5 aa, synthetic construct
GHRID

SEQ ID NO: 78 - protein, 5 aa, synthetic construct
GHRLD

SEQ ID NO: 79 - protein, 5 aa, synthetic construct
GHRMD

SEQ ID NO: 80 - protein, 5 aa, synthetic construct
GHRFD

SEQ ID NO: 81 - protein, 5 aa, synthetic construct
GHRAD

SEQ ID NO: 82 - protein, 5 aa, synthetic construct
PQRID

SEQ ID NO: 83 - protein, 5 aa, synthetic construct
PQRLD

SEQ ID NO: 84 - protein, 5 aa, synthetic construct
PQRMD

SEQ ID NO: 85 - protein, 5 aa, synthetic construct
PQRFD

SEQ ID NO: 86 - protein, 5 aa, synthetic construct
PQRAD

SEQ ID NO: 87 - protein, 5 aa, synthetic construct
PHRID

SEQ ID NO: 88 - protein, 5 aa, synthetic construct
PHRLD

SEQ ID NO: 89 - protein, 5 aa, synthetic construct
PHRMD

SEQ ID NO: 90 - protein, 5 aa, synthetic construct
PHRFD

SEQ ID NO: 91 - protein, 5 aa, synthetic construct
PHRAD

SEQ ID NO: 92 - protein, 5 aa, synthetic construct
AQRID

SEQ ID NO: 93 - protein, 5 aa, synthetic construct
AQRLD

SEQ ID NO: 94 - protein, 5 aa, synthetic construct
AQRMD

SEQ ID NO: 95 - protein, 5 aa, synthetic construct
AQRMD

SEQ ID NO: 96 - protein, 5 aa, synthetic construct
PHKMD

SEQ ID NO: 97 - protein, 5 aa, synthetic construct
PHKFD

SEQ ID NO: 98 - protein, 5 aa, synthetic construct
PHKAD

SEQ ID NO: 99 - protein, 5 aa, synthetic construct
AQKID

SEQ ID NO: 100 - protein, 5 aa, synthetic construct
AQKLD

SEQ ID NO: 101 - protein, 5 aa, synthetic construct
AQKMD

SEQ ID NO: 102 - protein, 5 aa, synthetic construct
AQKFD

SEQ ID NO: 103 - protein, 5 aa, synthetic construct
AQKAD

SEQ ID NO: 104 - protein, 5 aa, synthetic construct
AHKID

SEQ ID NO: 105 - protein, 5 aa, synthetic construct
AHKLD

SEQ ID NO: 106 - protein, 5 aa, synthetic construct
AHKMD

SEQ ID NO: 107 - protein, 5 aa, synthetic construct
AHKFD

SEQ ID NO: 108 - protein, 5 aa, synthetic construct
AHKAD

SEQ ID NO: 109 - protein, 7 aa, synthetic construct
GNRVDAH

SEQ ID NO: 110 - protein, 7 aa, synthetic construct
GNRVDDF

SEQ ID NO: 111-112 - skipped (2 entries)

SEQ ID NO: 113 - DNA, 7272 bp, synthetic construct
  source 1..7272: mol_type = other DNA
gcgcgccgatatcgttaacgccccgcgccggccgctctagaactagtggatcccccggaagatcagaagttcctattccgaagttcctattctctagaaagtataggaacttctgatctgcgcagccgccatgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacaacctctctgagggcattcgcgagtggtgggcgctgaaacctggagccccgaagcccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctgcaggcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagccatcaccccagcgttctccagactcctctacgggcatcggcaagaaaggccaacagcccgccagaaaaagactcaattttggtcagactggcgactcagagtcagttccagaccctcaacctctcggagaacctccagcagcgccctctggtgtgggacctaatacaatggctgcaggcggtggcgcaccaatggcagacaataacgaaggcgccgacggagtgggtagttcctcgggaaattggcattgcgattccacatggctgggcgacagagtcatcaccaccagcacccgaacctgggccctgcccacctacaacaaccacctctacaagcaaatctccaacgggacatcgggaggagccaccaacgacaacacctacttcggctacagcaccccctgggggtattttgactttaacagattccactgccacttttcaccacgtgactggcagcgactcatcaacaacaactggggattccggcccaagagactcagcttcaagctcttcaacatccaggtcaaggaggtcacgcagaatgaaggcaccaagaccatcgccaataacctcaccagcaccatccaggtgtttacggactcggagtaccagctgccgtacgttctcggctctgcccaccagggctgcctgcctccgttcccggcggacgtgttcatgattccccagtacggctacctaacactcaacaacggtagtcaggccgtgggacgctcctccttctactgcctggaatactttccttcgcagatgctgagaaccggcaacaacttccagtttacttacaccttcgaggacgtgcctttccacagcagctacgcccacagccagagcttggaccggctgatgaatcctctgattgaccagtacctgtactacttgtctcggactcaaacaacaggaggcacggcaaatacgcagactctgggcttcagccaaggtgggcctaatacaatggccaatcaggcaaagaactggctgccaggaccctgttaccgccaacaacgcgtctcaacgacaaccgggcaaaacaacaatagcaactttgcctggactgctgggaccaaataccatctgaatggaagaaattcattggctaatcctggcatcgctatggcaacacacaaagacgacgaggagcgtttttttcccagtaacgggatcctgatttttggcaaacaaaatgctgccagagacaatgcggattacagcgatgtcatgctcaccagcgaggaagaaatcaaaaccactaaccctgtggctacagaggaatacggtatcgtggcagataacttgcagcagcaaaacacggctcctcaaattggaactgtcaacagccagggggccttacccggtatggtctggcagaaccgggacgtgtacctgcagggtcccatctgggccaagattcctcacacggacggcaacttccacccgtctccgctgatgggcggctttggcctgaaacatcctccgcctcagatcctgatcaagaacacgcctgtacctgcggatcctccgaccaccttcaaccagtcaaagctgaactctttcatcacgcaatacagcaccggacaggtcagcgtggaaattgaatgggagctgcagaaggaaaacagcaagcgctggaaccccgagatccagtacacctccaactactacaaatctacaagtgtggactttgctgttaatacagaaggcgtgtactctgaaccccgccccattggcacccgttacctcacccgtaatctgtaattgcctgttaatcaataaaccggttgattcgtttcagttgaactttggtctctgcgaagggcgaattcgtttaaacagcgggcggaggggtggagtcgtgacgtgaattacgtcatagggttagggaggtcctgtattagaggtcacgtgagtgttttgcgacattttgcgacaccatgtggtctcgctgggggggggggcccgagtgagcacgcagggtctccattttgaagcgggaggtttgaacgagcgctggcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggaaattgtaagcgttaatattttgttaaaattcgcgttaaatttttgttaaatcagctcatttttttaaccaataggccgaaatcggcaaaatcccttataaatcaaaagaatagaccgagatagggttgagtgttgttccagtttggaacaagagtccactattaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaa

SEQ ID NO: 114 - DNA, 7314 bp, synthetic construct
  source 1..7314: mol_type = other DNA
gcgcgccgatatcgttaacgccccgcgccggccgctctagaggtcctgtattagaggtcacgtgagtgttttgcgacattttgcgacaccatgtggtcacgctgggtatttaagcccgagtgagcacgcagggtctccattttgaagcgggaggtttgaacgcgcagccaccacggcggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacaacctctctgagggcattcgcgagtggtgggcgctgaaacctggagccccgaagcccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctgcaggcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagccatcaccccagcgttctccagactcctctacgggcatcggcaagaaaggccaacagcccgccagaaaaagactcaattttggtcagactggcgactcagagtcagttccagaccctcaacctctcggagaacctccagcagcgccctctggtgtgggacctaatacaatggctgcaggcggtggcgcaccaatggcagacaataacgaaggcgccgacggagtgggtagttcctcgggaaattggcattgcgattccacatggctgggcgacagagtcatcaccaccagcacccgaacctgggccctgcccacctacaacaaccacctctacaagcaaatctccaacgggacatcgggaggagccaccaacgacaacacctacttcggctacagcaccccctgggggtattttgactttaacagattccactgccacttttcaccacgtgactggcagcgactcatcaacaacaactggggattccggcccaagagactcagcttcaagctcttcaacatccaggtcaaggaggtcacgcagaatgaaggcaccaagaccatcgccaataacctcaccagcaccatccaggtgtttacggactcggagtaccagctgccgtacgttctcggctctgcccaccagggctgcctgcctccgttcccggcggacgtgttcatgattccccagtacggctacctaacactcaacaacggtagtcaggccgtgggacgctcctccttctactgcctggaatactttccttcgcagatgctgagaaccggcaacaacttccagtttacttacaccttcgaggacgtgcctttccacagcagctacgcccacagccagagcttggaccggctgatgaatcctctgattgaccagtacctgtactacttgtctcggactcaaacaacaggaggcacggcaaatacgcagactctgggcttcagccaaggtgggcctaatacaatggccaatcaggcaaagaactggctgccaggaccctgttaccgccaacaacgcgtctcaacgacaaccgggcaaaacaacaatagcaactttgcctggactgctgggaccaaataccatctgaatggaagaaattcattggctaatcctggcatcgctatggcaacacacaaagacgacgaggagcgtttttttcccagtaacgggatcctgatttttggcaaacaaaatgctgccagagacaatgcggattacagcgatgtcatgctcaccagcgaggaagaaatcaaaaccactaaccctgtggctacagaggaatacggtatcgtggcagataacttgcagcagcaaaacacggctcctcaaattggaactgtcaacagccagggggccttacccggtatggtctggcagaaccgggacgtgtacctgcagggtcccatctgggccaagattcctcacacggacggcaacttccacccgtctccgctgatgggcggctttggcctgaaacatcctccgcctcagatcctgatcaagaacacgcctgtacctgcggatcctccgaccaccttcaaccagtcaaagctgaactctttcatcacgcaatacagcaccggacaggtcagcgtggaaattgaatgggagctgcagaaggaaaacagcaagcgctggaaccccgagatccagtacacctccaactactacaaatctacaagtgtggactttgctgttaatacagaaggcgtgtactctgaaccccgccccattggcacccgttacctcacccgtaatctgtaattgcctgttaatcaataaaccggttgattcgtttcagttgaactttggtctctgcgaagggcgaattcgtttaaacagcgggcggaggggtggagtcgtgacgtgaattacgtcatagggttagggaggtcctgtattagaggtcacgtgagtgttttgcgacattttgcgacaccatgtggtctcgctgggggggggggcccgagtgagcacgcagggtctccattttgaagcgggaggtttgaacgagcgctggcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggaaattgtaagcgttaatattttgttaaaattcgcgttaaatttttgttaaatcagctcatttttttaaccaataggccgaaatcggcaaaatcccttataaatcaaaagaatagaccgagatagggttgagtgttgttccagtttggaacaagagtccactattaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaa

SEQ ID NO: 115 - DNA, 7480 bp, synthetic construct
  source 1..7480: mol_type = other DNA
gcgcgccgatatcgttaacgccccgcgccggccgctctagaggtcctgtattagaggtcacgtgagtgttttgcgacattttgcgacaccatgtggtcacgctgggtatttaagcccgagtgagcacgcagggtctccattttgaagcgggaggtttgaacgcgcagccaccacggcggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacaacctctctgagggcattcgcgagtggtgggcgctgaaacctggagccccgaagcccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctgcaggcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagccatcaccccagcgttctccagactcctctacgggcatcggcaagaaaggccaacagcccgccagaaaaagactcaattttggtcagactggcgactcagagtcagttccagaccctcaacctctcggagaacctccagcagcgccctctggtgtgggacctaatacaatggctgcaggcggtggcgcaccaatggcagacaataacgaaggcgccgacggagtgggtagttcctcgggaaattggcattgcgattccacatggctgggcgacagagtcatcaccaccagcacccgaacctgggccctgcccacctacaacaaccacctctacaagcaaatctccaacgggacatcgggaggagccaccaacgacaacacctacttcggctacagcaccccctgggggtattttgactttaacagattccactgccacttttcaccacgtgactggcagcgactcatcaacaacaactggggattccggcccaagagactcagcttcaagctcttcaacatccaggtcaaggaggtcacgcagaatgaaggcaccaagaccatcgccaataacctcaccagcaccatccaggtgtttacggactcggagtaccagctgccgtacgttctcggctctgcccaccagggctgcctgcctccgttcccggcggacgtgttcatgattccccagtacggctacctaacactcaacaacggtagtcaggccgtgggacgctcctccttctactgcctggaatactttccttcgcagatgctgagaaccggcaacaacttccagtttacttacaccttcgaggacgtgcctttccacagcagctacgcccacagccagagcttggaccggctgatgaatcctctgattgaccagtacctgtactacttgtctcggactcaaacaacaggaggcacggcaaatacgcagactctgggcttcagccaaggtgggcctaatacaatggccaatcaggcaaagaactggctgccaggaccctgttaccgccaacaacgcgtctcaacgacaaccgggcaaaacaacaatagcaactttgcctggactgctgggaccaaataccatctgaatggaagaaattcattggctaatcctggcatcgctatggcaacacacaaagacgacgaggagcgtttttttcccagtaacgggatcctgatttttggcaaacaaaatgctgccagagacaatgcggattacagcgatgtcatgctcaccagcgaggaagaaatcaaaaccactaaccctgtggctacagaggaatacggtatcgtggcagataacttgcagcagcaaaacacggctcctcaaattggaactgtcaacagccagggggccttacccggtatggtctggcagaaccgggacgtgtacctgcagggtcccatctgggccaagattcctcacacggacggcaacttccacccgtctccgctgatgggcggctttggcctgaaacatcctccgcctcagatcctgatcaagaacacgcctgtacctgcggatcctccgaccaccttcaaccagtcaaagctgaactctttcatcacgcaatacagcaccggacaggtcagcgtggaaattgaatgggagctgcagaaggaaaacagcaagcgctggaaccccgagatccagtacacctccaactactacaaatctacaagtgtggactttgctgttaatacagaaggcgtgtactctgaaccccgccccattggcacccgttacctcacccgtaatctgtaattgcctgttaatcaataaaccggttgattcgtttcagttgaactttggtctctgcgaagggcgaattcgtttaaacctgcaggactagaggtcctgtattagaggtcacgtgagtgttttgcgacattttgcgacaccatgtggtcacgctgggtatttaagcccgagtgagcacgcagggtctccattttgaagcgggaggtttgaacgcgcagccgccaagccgaattctgcagataaacagcgggcggaggggtggagtcgtgacgtgaattacgtcatagggttagggaggtcctgtattagaggtcacgtgagtgttttgcgacattttgcgacaccatgtggtctcgctgggggggggggcccgagtgagcacgcagggtctccattttgaagcgggaggtttgaacgagcgctggcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggaaattgtaagcgttaatattttgttaaaattcgcgttaaatttttgttaaatcagctcatttttttaaccaataggccgaaatcggcaaaatcccttataaatcaaaagaatagaccgagatagggttgagtgttgttccagtttggaacaagagtccactattaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaa

SEQ ID NO: 116 - DNA, 7480 bp, synthetic construct
  source 1..7480: mol_type = other DNA
gcgcgccgatatcgttaacgccccgcgccggccgctctagaggtcctgtattagaggtcacgtgagtgttttgcgacattttgcgacaccatgtggtcacgctgggtatttaagcccgagtgagcacgcagggtctccattttgaagcgggaggtttgaacgcgcagccaccacggcggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacaacctctctgagggcattcgcgagtggtgggcgctgaaacctggagccccgaagcccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctgcaggcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagccatcaccccagcgttctccagactcctctacgggcatcggcaagaaaggccaacagcccgccagaaaaagactcaattttggtcagactggcgactcagagtcagttccagaccctcaacctctcggagaacctccagcagcgccctctggtgtgggacctaatacaatggctgcaggcggtggcgcaccaatggcagacaataacgaaggcgccgacggagtgggtagttcctcgggaaattggcattgcgattccacatggctgggcgacagagtcatcaccaccagcacccgaacctgggccctgcccacctacaacaaccacctctacaagcaaatctccaacgggacatcgggaggagccaccaacgacaacacctacttcggctacagcaccccctgggggtattttgactttaacagattccactgccacttttcaccacgtgactggcagcgactcatcaacaacaactggggattccggcccaagagactcagcttcaagctcttcaacatccaggtcaaggaggtcacgcagaatgaaggcaccaagaccatcgccaataacctcaccagcaccatccaggtgtttacggactcggagtaccagctgccgtacgttctcggctctgcccaccagggctgcctgcctccgttcccggcggacgtgttcatgattccccagtacggctacctaacactcaacaacggtagtcaggccgtgggacgctcctccttctactgcctggaatactttccttcgcagatgctgagaaccggcaacaacttccagtttacttacaccttcgaggacgtgcctttccacagcagctacgcccacagccagagcttggaccggctgatgaatcctctgattgaccagtacctgtacttcttgtctcggactcaaacaacaggaggcacggcaaatacgcagactctgggcttcagccaaggtgggcctaatacaatggccaatcaggcaaagaactggctgccaggaccctgttaccgccaacaacgcgtctcaacggtaaccgggcaaaacaacaatagcaactttgcctggactgctgggaccaaataccatctgaatggaagaaattcattggctaatcctggcatcgctatggcaacacacaaagacgacgaggagcgtttttttcccagtaacgggatcctgatttttggcaaacaaaatgctgccagagacaatgcggattacagcgatgtcatgctcaccagcgaggaagaaatcaaaaccactaaccctgtggctacagaggaatacggtatcgtggcagataacttgcagcagcaaaacacggctcctcaaattggaactgtcaacagccagggggccttacccggtatggtctggcagaaccgggacgtgtacctgcagggtcccatctgggccaagattcctcacacggacggcaacttccacccgtctccgctgatgggcggctttggcctgaaacatcctccgcctcagatcctgatcaagaacacgcctgtacctgcggatcctccgaccaccttcaaccagtcaaagctgaactctttcatcacgcaatacagcaccggacaggtcagcgtggaaattgaatgggagctgcagaaggaaaacagcaagcgctggaaccccgagatccagtacacctccaactactacaaatctacaagtgtggactttgctgttaatacagaaggcgtgtactctgaaccccgccccattggcacccgtttcctcacccgtaatctgtaattgcctgttaatcaataaaccggttgattcgtttcagttgaactttggtctctgcgaagggcgaattcgtttaaacctgcaggactagaggtcctgtattagaggtcacgtgagtgttttgcgacattttgcgacaccatgtggtcacgctgggtatttaagcccgagtgagcacgcagggtctccattttgaagcgggaggtttgaacgcgcagccgccaagccgaattctgcagataaacagcgggcggaggggtggagtcgtgacgtgaattacgtcatagggttagggaggtcctgtattagaggtcacgtgagtgttttgcgacattttgcgacaccatgtggtctcgctgggggggggggcccgagtgagcacgcagggtctccattttgaagcgggaggtttgaacgagcgctggcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggaaattgtaagcgttaatattttgttaaaattcgcgttaaatttttgttaaatcagctcatttttttaaccaataggccgaaatcggcaaaatcccttataaatcaaaagaatagaccgagatagggttgagtgttgttccagtttggaacaagagtccactattaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaa

SEQ ID NO: 117 - DNA, 7407 bp, synthetic construct
  source 1..7407: mol_type = other DNA
ccgatatcgttaacgccccgcgccggccgctctagaactagtggatcccccggaagatcagaagttcctattccgaagttcctattctctagaaagtataggaacttctgatctgcgcagccgccatgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacaacctctctgagggcattcgcgagtggtgggcgctgaaacctggagccccgaagcccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctgcaggcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagccatcaccccagcgttctccagactcctctacgggcatcggcaagaaaggccaacagcccgccagaaaaagactcaattttggtcagactggcgactcagagtcagttccagaccctcaacctctcggagaacctccagcagcgccctctggtgtgggacctaatacaatggctgcaggcggtggcgcaccaatggcagacaataacgaaggcgccgacggagtgggtagttcctcgggaaattggcattgcgattccacatggctgggcgacagagtcatcaccaccagcacccgaacctgggccctgcccacctacaacaaccacctctacaagcaaatctccaacgggacatcgggaggagccaccaacgacaacacctacttcggctacagcaccccctgggggtattttgactttaacagattccactgccacttttcaccacgtgactggcagcgactcatcaacaacaactggggattccggcccaagagactcagcttcaagctcttcaacatccaggtcaaggaggtcacgcagaatgaaggcaccaagaccatcgccaataacctcaccagcaccatccaggtgtttacggactcggagtaccagctgccgtacgttctcggctctgcccaccagggctgcctgcctccgttcccggcggacgtgttcatgattccccagtacggctacctaacactcaacaacggtagtcaggccgtgggacgctcctccttctactgcctggaatactttccttcgcagatgctgagaaccggcaacaacttccagtttacttacaccttcgaggacgtgcctttccacagcagctacgcccacagccagagcttggaccggctgatgaatcctctgattgaccagtacctgtacttcttgtctcggactcaaacaacaggaggcacggcaaatacgcagactctgggcttcagccaaggtgggcctaatacaatggccaatcaggcaaagaactggctgccaggaccctgttaccgccaacaacgcgtctcaacggtaaccgggcaaaacaacaatagcaactttgcctggactgctgggaccaaataccatctgaatggaagaaattcattggctaatcctggcatcgctatggcaacacacaaagacgacgaggagcgtttttttcccagtaacgggatcctgatttttggcaaacaaaatgctgccagagacaatgcggattacagcgatgtcatgctcaccagcgaggaagaaatcaaaaccactaaccctgtggctacagaggaatacggtatcgtggcagataacttgcagcagcaaaacagatctcgcggtgatcttaagacggctcctcaaattggaactgtcaacagccagggggccttacccggtatggtctggcagaaccgggacgtgtacctgcagggtcccatctgggccaagattcctcacacggacggcaacttccacccgtctccgctgatgggcggctttggcctgaaacatcctccgcctcagatcctgatcaagaacacgcctgtacctgcggatcctccgaccaccttcaaccagtcaaagctgaactctttcatcacgcaatacagcaccggacaggtcagcgtggaaattgaatgggagctgcagaaggaaaacagcaagcgctggaaccccgagatccagtacacctccaactactacaaatctacaagtgtggactttgctgttaatacagaaggcgtgtactctgaaccccgccccattggcacccgtttcctcacccgtaatctgtaattgcctgttaatcaataaaccggttgattcgtttcagttgaactttggtctctgcgaagggcgaattcgtttaaacagcgggcggaggggtggagtcgtgacgtgaattacgtcatagggttagggaggtcctgtattagaggtcacgtgagtgttttgcgacattttgcgacaccatgtggtctcgctgggggggggggcccgagtgagcacgcagggtctccattttgaagcgggaggtttgaacgagcgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtcctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcg

SEQ ID NO: 118 - DNA, 133 bp, synthetic construct
  source 1..133: mol_type = other DNA
gaggtcctgtattagaggtcacgtgagtgttttgcgacattttgcgacaccatgtggtcacgctgggtatttaagcccgagtgagcacgcagggtctccattttgaagcgggaggtttgaacgcgcagccacc

SEQ ID NO: 119 - DNA, 133 bp, synthetic construct
  source 1..133: mol_type = other DNA
gaggtcctgtattagaggtcacgtgagtgttttgcgacattttgcgacaccatgtggtcacgctgggtatttaagcccgagtgagcacgcagggtctccattttgaagcgggaggtttgaacgcgcagccgcc

SEQ ID NO: 120 - DNA, 2217 bp, Adeno-associated dependoparvovirus A
  source 1..2217: mol_type = other DNA
atggctgccgatggttatcttccagattggctcgaggacaacctctctgagggcattcgcgagtggtgggcgctgaaacctggagccccgaagcccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctgcaggcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagccatcaccccagcgttctccagactcctctacgggcatcggcaagaaaggccaacagcccgccagaaaaagactcaattttggtcagactggcgactcagagtcagttccagaccctcaacctctcggagaacctccagcagcgccctctggtgtgggacctaatacaatggctgcaggcggtggcgcaccaatggcagacaataacgaaggcgccgacggagtgggtagttcctcgggaaattggcattgcgattccacatggctgggcgacagagtcatcaccaccagcacccgaacctgggccctgcccacctacaacaaccacctctacaagcaaatctccaacgggacatcgggaggagccaccaacgacaacacctacttcggctacagcaccccctgggggtattttgactttaacagattccactgccacttttcaccacgtgactggcagcgactcatcaacaacaactggggattccggcccaagagactcagcttcaagctcttcaacatccaggtcaaggaggtcacgcagaatgaaggcaccaagaccatcgccaataacctcaccagcaccatccaggtgtttacggactcggagtaccagctgccgtacgttctcggctctgcccaccagggctgcctgcctccgttcccggcggacgtgttcatgattccccagtacggctacctaacactcaacaacggtagtcaggccgtgggacgctcctccttctactgcctggaatactttccttcgcagatgctgagaaccggcaacaacttccagtttacttacaccttcgaggacgtgcctttccacagcagctacgcccacagccagagcttggaccggctgatgaatcctctgattgaccagtacctgtactacttgtctcggactcaaacaacaggaggcacggcaaatacgcagactctgggcttcagccaaggtgggcctaatacaatggccaatcaggcaaagaactggctgccaggaccctgttaccgccaacaacgcgtctcaacgacaaccgggcaaaacaacaatagcaactttgcctggactgctgggaccaaataccatctgaatggaagaaattcattggctaatcctggcatcgctatggcaacacacaaagacgacgaggagcgtttttttcccagtaacgggatcctgatttttggcaaacaaaatgctgccagagacaatgcggattacagcgatgtcatgctcaccagcgaggaagaaatcaaaaccactaaccctgtggctacagaggaatacggtatcgtggcagataacttgcagcagcaaaacacggctcctcaaattggaactgtcaacagccagggggccttacccggtatggtctggcagaaccgggacgtgtacctgcagggtcccatctgggccaagattcctcacacggacggcaacttccacccgtctccgctgatgggcggctttggcctgaaacatcctccgcctcagatcctgatcaagaacacgcctgtacctgcggatcctccgaccaccttcaaccagtcaaagctgaactctttcatcacgcaatacagcaccggacaggtcagcgtggaaattgaatgggagctgcagaaggaaaacagcaagcgctggaaccccgagatccagtacacctccaactactacaaatctacaagtgtggactttgctgttaatacagaaggcgtgtactctgaaccccgccccattggcacccgttacctcacccgtaatctgtaa

SEQ ID NO: 121 - protein, 738 aa, Adeno-associated dependoparvovirus A
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 122 - DNA, 1740 bp, Adeno-associated dependoparvovirus A
  source 1..1740: mol_type = other DNA
atggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataa

SEQ ID NO: 123 - protein, 621 aa, Adeno-associated dependoparvovirus A
MPGFYEIVIKVPSDLDEHLPGISDSFVNWVAEKEWELPPDSDMDLNLIEQAPLTVAEKLQRDFLTEWRRVSKAPEALFFVQFEKGESYFHMHVLVETTGVKSMVLGRFLSQIREKLIQRIYRGIEPTLPNWFAVTKTRNGAGGGNKVVDECYIPNYLLPKTQPELQWAWTNMEQYLSACLNLTERKRLVAQHLTHVSQTQEQNKENQNPNSDAPVIRSKTSARYMELVGWLVDKGITSEKQWIQEDQASYISFNAASNSRSQIKAALDNAGKIMSLTKTAPDYLVGQQPVEDISSNRIYKILELNGYDPQYAASVFLGWATKKFGKRNTIWLFGPATTGKTNIAEAIAHTVPFYGCVNWTNENFPFNDCVDKMVIWWEEGKMTAKVVESAKAILGGSKVRVDQKCKSSAQIDPTPVIVTSNTNMCAVIDGNSTTFEHQQPLQDRMFKFELTRRLDHDFGKVTKQEVKDFFRWAKDHVVEVEHEFYVKKGGAKKRPAPSDADISEPKRVRESVAQPSTSDAEASINYADRYQNKCSRHVGMNLMLFPCRQCERMNQNSNICFTHGQKDCLECFPVSESQPVSVVKKAYQKLCYIHHIMGKVPDACTACDLVNVDLDDCIFEQ

SEQ ID NO: 124 - DNA, 2217 bp, synthetic construct
  source 1..2217: mol_type = other DNA
atggctgccgatggttatcttccagattggctcgaggacaacctctctgagggcattcgcgagtggtgggcgctgaaacctggagccccgaagcccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctgcaggcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagccatcaccccagcgttctccagactcctctacgggcatcggcaagaaaggccaacagcccgccagaaaaagactcaattttggtcagactggcgactcagagtcagttccagaccctcaacctctcggagaacctccagcagcgccctctggtgtgggacctaatacaatggctgcaggcggtggcgcaccaatggcagacaataacgaaggcgccgacggagtgggtagttcctcgggaaattggcattgcgattccacatggctgggcgacagagtcatcaccaccagcacccgaacctgggccctgcccacctacaacaaccacctctacaagcaaatctccaacgggacatcgggaggagccaccaacgacaacacctacttcggctacagcaccccctgggggtattttgactttaacagattccactgccacttttcaccacgtgactggcagcgactcatcaacaacaactggggattccggcccaagagactcagcttcaagctcttcaacatccaggtcaaggaggtcacgcagaatgaaggcaccaagaccatcgccaataacctcaccagcaccatccaggtgtttacggactcggagtaccagctgccgtacgttctcggctctgcccaccagggctgcctgcctccgttcccggcggacgtgttcatgattccccagtacggctacctaacactcaacaacggtagtcaggccgtgggacgctcctccttctactgcctggaatactttccttcgcagatgctgagaaccggcaacaacttccagtttacttacaccttcgaggacgtgcctttccacagcagctacgcccacagccagagcttggaccggctgatgaatcctctgattgaccagtacctgtacttcttgtctcggactcaaacaacaggaggcacggcaaatacgcagactctgggcttcagccaaggtgggcctaatacaatggccaatcaggcaaagaactggctgccaggaccctgttaccgccaacaacgcgtctcaacggtaaccgggcaaaacaacaatagcaactttgcctggactgctgggaccaaataccatctgaatggaagaaattcattggctaatcctggcatcgctatggcaacacacaaagacgacgaggagcgtttttttcccagtaacgggatcctgatttttggcaaacaaaatgctgccagagacaatgcggattacagcgatgtcatgctcaccagcgaggaagaaatcaaaaccactaaccctgtggctacagaggaatacggtatcgtggcagataacttgcagcagcaaaacacggctcctcaaattggaactgtcaacagccagggggccttacccggtatggtctggcagaaccgggacgtgtacctgcagggtcccatctgggccaagattcctcacacggacggcaacttccacccgtctccgctgatgggcggctttggcctgaaacatcctccgcctcagatcctgatcaagaacacgcctgtacctgcggatcctccgaccaccttcaaccagtcaaagctgaactctttcatcacgcaatacagcaccggacaggtcagcgtggaaattgaatgggagctgcagaaggaaaacagcaagcgctggaaccccgagatccagtacacctccaactactacaaatctacaagtgtggactttgctgttaatacagaaggcgtgtactctgaaccccgccccattggcacccgtttcctcacccgtaatctgtaa

SEQ ID NO: 125 - protein, 738 aa, synthetic construct
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYFLSRTQTTGGTANTQTLGFSQGGPNTMANQAKNWLPGPCYRQQRVSTVTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRFLTRNL

SEQ ID NO: 126 - DNA, 2238 bp, synthetic construct
  source 1..2238: mol_type = other DNA
atggctgccgatggttatcttccagattggctcgaggacaacctctctgagggcattcgcgagtggtgggcgctgaaacctggagccccgaagcccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctgcaggcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagccatcaccccagcgttctccagactcctctacgggcatcggcaagaaaggccaacagcccgccagaaaaagactcaattttggtcagactggcgactcagagtcagttccagaccctcaacctctcggagaacctccagcagcgccctctggtgtgggacctaatacaatggctgcaggcggtggcgcaccaatggcagacaataacgaaggcgccgacggagtgggtagttcctcgggaaattggcattgcgattccacatggctgggcgacagagtcatcaccaccagcacccgaacctgggccctgcccacctacaacaaccacctctacaagcaaatctccaacgggacatcgggaggagccaccaacgacaacacctacttcggctacagcaccccctgggggtattttgactttaacagattccactgccacttttcaccacgtgactggcagcgactcatcaacaacaactggggattccggcccaagagactcagcttcaagctcttcaacatccaggtcaaggaggtcacgcagaatgaaggcaccaagaccatcgccaataacctcaccagcaccatccaggtgtttacggactcggagtaccagctgccgtacgttctcggctctgcccaccagggctgcctgcctccgttcccggcggacgtgttcatgattccccagtacggctacctaacactcaacaacggtagtcaggccgtgggacgctcctccttctactgcctggaatactttccttcgcagatgctgagaaccggcaacaacttccagtttacttacaccttcgaggacgtgcctttccacagcagctacgcccacagccagagcttggaccggctgatgaatcctctgattgaccagtacctgtacttcttgtctcggactcaaacaacaggaggcacggcaaatacgcagactctgggcttcagccaaggtgggcctaatacaatggccaatcaggcaaagaactggctgccaggaccctgttaccgccaacaacgcgtctcaacggtaaccgggcaaaacaacaatagcaactttgcctggactgctgggaccaaataccatctgaatggaagaaattcattggctaatcctggcatcgctatggcaacacacaaagacgacgaggagcgtttttttcccagtaacgggatcctgatttttggcaaacaaaatgctgccagagacaatgcggattacagcgatgtcatgctcaccagcgaggaagaaatcaaaaccactaaccctgtggctacagaggaatacggtatcgtggcagataacttgcagcagcaaaacagatctcgcggtgatcttaagacggctcctcaaattggaactgtcaacagccagggggccttacccggtatggtctggcagaaccgggacgtgtacctgcagggtcccatctgggccaagattcctcacacggacggcaacttccacccgtctccgctgatgggcggctttggcctgaaacatcctccgcctcagatcctgatcaagaacacgcctgtacctgcggatcctccgaccaccttcaaccagtcaaagctgaactctttcatcacgcaatacagcaccggacaggtcagcgtggaaattgaatgggagctgcagaaggaaaacagcaagcgctggaaccccgagatccagtacacctccaactactacaaatctacaagtgtggactttgctgttaatacagaaggcgtgtactctgaaccccgccccattggcacccgtttcctcacccgtaatctgtaa

SEQ ID NO: 127 - protein, 745 aa, synthetic construct
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYFLSRTQTTGGTANTQTLGFSQGGPNTMANQAKNWLPGPCYRQQRVSTVTGQNNNSNFAWTAGTKYHLNGRNSLANPGIAMATHKDDEERFFPSNGILIFGKQNAARDNADYSDVMLTSEEEIKTTNPVATEEYGIVADNLQQQNRSRGDLKTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRFLTRNL

SEQ ID NO: 128 - DNA, 37 bp, synthetic construct
  source 1..37: mol_type = other DNA
accggtggcaaccgggtggacgcccacggtctttcga

SEQ ID NO: 129 - protein, 12 aa, synthetic construct
TGGNRVDAHGLS

SEQ ID NO: 130 - protein, 146 aa, synthetic construct
TQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNGNRVDTAPQIGTVNS

SEQ ID NO: 131 - protein, 151 aa, synthetic construct
TQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTGGNRVDGLSTAPQIGTVNS

SEQ ID NO: 132 - protein, 148 aa, synthetic construct
TQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNGNRVDAHTAPQIGTVNS

SEQ ID NO: 133 - protein, 153 aa, synthetic construct
TQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTGGNRVDAHGLSTAPQIGTVNS

SEQ ID NO: 134 - DNA, 2253 bp, synthetic construct
  source 1..2253: mol_type = other DNA
atggctgccgatggttatcttccagattggctcgaggacaacctctctgagggcattcgcgagtggtgggcgctgaaacctggagccccgaagcccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctgcaggcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagccatcaccccagcgttctccagactcctctacgggcatcggcaagaaaggccaacagcccgccagaaaaagactcaattttggtcagactggcgactcagagtcagttccagaccctcaacctctcggagaacctccagcagcgccctctggtgtgggacctaatacaatggctgcaggcggtggcgcaccaatggcagacaataacgaaggcgccgacggagtgggtagttcctcgggaaattggcattgcgattccacatggctgggcgacagagtcatcaccaccagcacccgaacctgggccctgcccacctacaacaaccacctctacaagcaaatctccaacgggacatcgggaggagccaccaacgacaacacctacttcggctacagcaccccctgggggtattttgactttaacagattccactgccacttttcaccacgtgactggcagcgactcatcaacaacaactggggattccggcccaagagactcagcttcaagctcttcaacatccaggtcaaggaggtcacgcagaatgaaggcaccaagaccatcgccaataacctcaccagcaccatccaggtgtttacggactcggagtaccagctgccgtacgttctcggctctgcccaccagggctgcctgcctccgttcccggcggacgtgttcatgattccccagtacggctacctaacactcaacaacggtagtcaggccgtgggacgctcctccttctactgcctggaatactttccttcgcagatgctgagaaccggcaacaacttccagtttacttacaccttcgaggacgtgcctttccacagcagctacgcccacagccagagcttggaccggctgatgaatcctctgattgaccagtacctgtacttcttgtctcggactcaaacaacaggaggcacggcaaatacgcagactctgggcttcagccaaggtgggcctaatacaatggccaatcaggcaaagaactggctgccaggaccctgttaccgccaacaacgcgtctcaacggtaaccgggcaaaacaacaatagcaactttgcctggactgctgggaccaaataccatctgaatggaagaaattcattggctaatcctggcatcgctatggcaacacacaaagacgacgaggagcgtttttttcccagtaacgggatcctgatttttggcaaacaaaatgctgccagagacaatgcggattacagcgatgtcatgctcaccagcgaggaagaaatcaaaaccactaaccctgtggctacagaggaatacggtatcgtggcagataacttgcagcagcaaaacaccggtggcaaccgggtggacgcccacggtctttcgacggctcctcaaattggaactgtcaacagccagggggccttacccggtatggtctggcagaaccgggacgtgtacctgcagggtcccatctgggccaagattcctcacacggacggcaacttccacccgtctccgctgatgggcggctttggcctgaaacatcctccgcctcagatcctgatcaagaacacgcctgtacctgcggatcctccgaccaccttcaaccagtcaaagctgaactctttcatcacgcaatacagcaccggacaggtcagcgtggaaattgaatgggagctgcagaaggaaaacagcaagcgctggaaccccgagatccagtacacctccaactactacaaatctacaagtgtggactttgctgttaatacagaaggcgtgtactctgaaccccgccccattggcacccgtttcctcacccgtaatctgtaa

SEQ ID NO: 135 - protein, 750 aa, synthetic construct
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYFLSRTQTTGGTANTQTLGFSQGGPNTMANQAKNWLPGPCYRQQRVSTVTGQNNNSNFAWTAGTKYHLNGRNSLANPGIAMATHKDDEERFFPSNGILIFGKQNAARDNADYSDVMLTSEEEIKTTNPVATEEYGIVADNLQQQNTGGNRVDAHGLSTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRFLTRNL

SEQ ID NO: 136 - DNA, 7516 bp, synthetic construct
  source 1..7516: mol_type = other DNA
gcgcgccgatatcgttaacgccccgcgccggccgctctagaggtcctgtattagaggtcacgtgagtgttttgcgacattttgcgacaccatgtggtcacgctgggtatttaagcccgagtgagcacgcagggtctccattttgaagcgggaggtttgaacgcgcagccaccacggcggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacaacctctctgagggcattcgcgagtggtgggcgctgaaacctggagccccgaagcccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctgcaggcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagccatcaccccagcgttctccagactcctctacgggcatcggcaagaaaggccaacagcccgccagaaaaagactcaattttggtcagactggcgactcagagtcagttccagaccctcaacctctcggagaacctccagcagcgccctctggtgtgggacctaatacaatggctgcaggcggtggcgcaccaatggcagacaataacgaaggcgccgacggagtgggtagttcctcgggaaattggcattgcgattccacatggctgggcgacagagtcatcaccaccagcacccgaacctgggccctgcccacctacaacaaccacctctacaagcaaatctccaacgggacatcgggaggagccaccaacgacaacacctacttcggctacagcaccccctgggggtattttgactttaacagattccactgccacttttcaccacgtgactggcagcgactcatcaacaacaactggggattccggcccaagagactcagcttcaagctcttcaacatccaggtcaaggaggtcacgcagaatgaaggcaccaagaccatcgccaataacctcaccagcaccatccaggtgtttacggactcggagtaccagctgccgtacgttctcggctctgcccaccagggctgcctgcctccgttcccggcggacgtgttcatgattccccagtacggctacctaacactcaacaacggtagtcaggccgtgggacgctcctccttctactgcctggaatactttccttcgcagatgctgagaaccggcaacaacttccagtttacttacaccttcgaggacgtgcctttccacagcagctacgcccacagccagagcttggaccggctgatgaatcctctgattgaccagtacctgtacttcttgtctcggactcaaacaacaggaggcacggcaaatacgcagactctgggcttcagccaaggtgggcctaatacaatggccaatcaggcaaagaactggctgccaggaccctgttaccgccaacaacgcgtctcaacggtaaccgggcaaaacaacaatagcaactttgcctggactgctgggaccaaataccatctgaatggaagaaattcattggctaatcctggcatcgctatggcaacacacaaagacgacgaggagcgtttttttcccagtaacgggatcctgatttttggcaaacaaaatgctgccagagacaatgcggattacagcgatgtcatgctcaccagcgaggaagaaatcaaaaccactaaccctgtggctacagaggaatacggtatcgtggcagataacttgcagcagcaaaacaccggtggcaaccgggtggacgcccacggtctttcgacggctcctcaaattggaactgtcaacagccagggggccttacccggtatggtctggcagaaccgggacgtgtacctgcagggtcccatctgggccaagattcctcacacggacggcaacttccacccgtctccgctgatgggcggctttggcctgaaacatcctccgcctcagatcctgatcaagaacacgcctgtacctgcggatcctccgaccaccttcaaccagtcaaagctgaactctttcatcacgcaatacagcaccggacaggtcagcgtggaaattgaatgggagctgcagaaggaaaacagcaagcgctggaaccccgagatccagtacacctccaactactacaaatctacaagtgtggactttgctgttaatacagaaggcgtgtactctgaaccccgccccattggcacccgtttcctcacccgtaatctgtaattgcctgttaatcaataaaccggttgattcgtttcagttgaactttggtctctgcgaagggcgaattcgtttaaacctgcaggactagaggtcctgtattagaggtcacgtgagtgttttgcgacattttgcgacaccatgtggtcacgctgggtatttaagcccgagtgagcacgcagggtctccattttgaagcgggaggtttgaacgcgcagccgccaagccgaattctgcagataaacagcgggcggaggggtggagtcgtgacgtgaattacgtcatagggttagggaggtcctgtattagaggtcacgtgagtgttttgcgacattttgcgacaccatgtggtctcgctgggggggggggcccgagtgagcacgcagggtctccattttgaagcgggaggtttgaacgagcgctggcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggaaattgtaagcgttaatattttgttaaaattcgcgttaaatttttgttaaatcagctcatttttttaaccaataggccgaaatcggcaaaatcccttataaatcaaaagaatagaccgagatagggttgagtgttgttccagtttggaacaagagtccactattaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaa

SEQ ID NO: 137 - DNA, 37 bp, synthetic construct
  source 1..37: mol_type = other DNA
accggtggcaaccgggtggacgacttcggtctttcga

SEQ ID NO: 138 - protein, 12 aa, synthetic construct
TGGNRVDDFGLS

SEQ ID NO: 139 - protein, 148 aa, synthetic construct
TQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNGNRVDDFTAPQIGTVNS

SEQ ID NO: 140 - protein, 153 aa, synthetic construct
TQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTGGNRVDDFGLSTAPQIGTVNS

SEQ ID NO: 141 - DNA, 2253 bp, synthetic construct
  source 1..2253: mol_type = other DNA
atggctgccgatggttatcttccagattggctcgaggacaacctctctgagggcattcgcgagtggtgggcgctgaaacctggagccccgaagcccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctgcaggcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagccatcaccccagcgttctccagactcctctacgggcatcggcaagaaaggccaacagcccgccagaaaaagactcaattttggtcagactggcgactcagagtcagttccagaccctcaacctctcggagaacctccagcagcgccctctggtgtgggacctaatacaatggctgcaggcggtggcgcaccaatggcagacaataacgaaggcgccgacggagtgggtagttcctcgggaaattggcattgcgattccacatggctgggcgacagagtcatcaccaccagcacccgaacctgggccctgcccacctacaacaaccacctctacaagcaaatctccaacgggacatcgggaggagccaccaacgacaacacctacttcggctacagcaccccctgggggtattttgactttaacagattccactgccacttttcaccacgtgactggcagcgactcatcaacaacaactggggattccggcccaagagactcagcttcaagctcttcaacatccaggtcaaggaggtcacgcagaatgaaggcaccaagaccatcgccaataacctcaccagcaccatccaggtgtttacggactcggagtaccagctgccgtacgttctcggctctgcccaccagggctgcctgcctccgttcccggcggacgtgttcatgattccccagtacggctacctaacactcaacaacggtagtcaggccgtgggacgctcctccttctactgcctggaatactttccttcgcagatgctgagaaccggcaacaacttccagtttacttacaccttcgaggacgtgcctttccacagcagctacgcccacagccagagcttggaccggctgatgaatcctctgattgaccagtacctgtacttcttgtctcggactcaaacaacaggaggcacggcaaatacgcagactctgggcttcagccaaggtgggcctaatacaatggccaatcaggcaaagaactggctgccaggaccctgttaccgccaacaacgcgtctcaacggtaaccgggcaaaacaacaatagcaactttgcctggactgctgggaccaaataccatctgaatggaagaaattcattggctaatcctggcatcgctatggcaacacacaaagacgacgaggagcgtttttttcccagtaacgggatcctgatttttggcaaacaaaatgctgccagagacaatgcggattacagcgatgtcatgctcaccagcgaggaagaaatcaaaaccactaaccctgtggctacagaggaatacggtatcgtggcagataacttgcagcagcaaaacaccggtggcaaccgggtggacgacttcggtctttcgacggctcctcaaattggaactgtcaacagccagggggccttacccggtatggtctggcagaaccgggacgtgtacctgcagggtcccatctgggccaagattcctcacacggacggcaacttccacccgtctccgctgatgggcggctttggcctgaaacatcctccgcctcagatcctgatcaagaacacgcctgtacctgcggatcctccgaccaccttcaaccagtcaaagctgaactctttcatcacgcaatacagcaccggacaggtcagcgtggaaattgaatgggagctgcagaaggaaaacagcaagcgctggaaccccgagatccagtacacctccaactactacaaatctacaagtgtggactttgctgttaatacagaaggcgtgtactctgaaccccgccccattggcacccgtttcctcacccgtaatctgtaa

SEQ ID NO: 142 - protein, 750 aa, synthetic construct
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYFLSRTQTTGGTANTQTLGFSQGGPNTMANQAKNWLPGPCYRQQRVSTVTGQNNNSNFAWTAGTKYHLNGRNSLANPGIAMATHKDDEERFFPSNGILIFGKQNAARDNADYSDVMLTSEEEIKTTNPVATEEYGIVADNLQQQNTGGNRVDDFGLSTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRFLTRNL

SEQ ID NO: 143 - DNA, 7516 bp, synthetic construct
  source 1..7516: mol_type = other DNA
gcgcgccgatatcgttaacgccccgcgccggccgctctagaggtcctgtattagaggtcacgtgagtgttttgcgacattttgcgacaccatgtggtcacgctgggtatttaagcccgagtgagcacgcagggtctccattttgaagcgggaggtttgaacgcgcagccaccacggcggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacaacctctctgagggcattcgcgagtggtgggcgctgaaacctggagccccgaagcccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctgcaggcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagccatcaccccagcgttctccagactcctctacgggcatcggcaagaaaggccaacagcccgccagaaaaagactcaattttggtcagactggcgactcagagtcagttccagaccctcaacctctcggagaacctccagcagcgccctctggtgtgggacctaatacaatggctgcaggcggtggcgcaccaatggcagacaataacgaaggcgccgacggagtgggtagttcctcgggaaattggcattgcgattccacatggctgggcgacagagtcatcaccaccagcacccgaacctgggccctgcccacctacaacaaccacctctacaagcaaatctccaacgggacatcgggaggagccaccaacgacaacacctacttcggctacagcaccccctgggggtattttgactttaacagattccactgccacttttcaccacgtgactggcagcgactcatcaacaacaactggggattccggcccaagagactcagcttcaagctcttcaacatccaggtcaaggaggtcacgcagaatgaaggcaccaagaccatcgccaataacctcaccagcaccatccaggtgtttacggactcggagtaccagctgccgtacgttctcggctctgcccaccagggctgcctgcctccgttcccggcggacgtgttcatgattccccagtacggctacctaacactcaacaacggtagtcaggccgtgggacgctcctccttctactgcctggaatactttccttcgcagatgctgagaaccggcaacaacttccagtttacttacaccttcgaggacgtgcctttccacagcagctacgcccacagccagagcttggaccggctgatgaatcctctgattgaccagtacctgtacttcttgtctcggactcaaacaacaggaggcacggcaaatacgcagactctgggcttcagccaaggtgggcctaatacaatggccaatcaggcaaagaactggctgccaggaccctgttaccgccaacaacgcgtctcaacggtaaccgggcaaaacaacaatagcaactttgcctggactgctgggaccaaataccatctgaatggaagaaattcattggctaatcctggcatcgctatggcaacacacaaagacgacgaggagcgtttttttcccagtaacgggatcctgatttttggcaaacaaaatgctgccagagacaatgcggattacagcgatgtcatgctcaccagcgaggaagaaatcaaaaccactaaccctgtggctacagaggaatacggtatcgtggcagataacttgcagcagcaaaacaccggtggcaaccgggtggacgacttcggtctttcgacggctcctcaaattggaactgtcaacagccagggggccttacccggtatggtctggcagaaccgggacgtgtacctgcagggtcccatctgggccaagattcctcacacggacggcaacttccacccgtctccgctgatgggcggctttggcctgaaacatcctccgcctcagatcctgatcaagaacacgcctgtacctgcggatcctccgaccaccttcaaccagtcaaagctgaactctttcatcacgcaatacagcaccggacaggtcagcgtggaaattgaatgggagctgcagaaggaaaacagcaagcgctggaaccccgagatccagtacacctccaactactacaaatctacaagtgtggactttgctgttaatacagaaggcgtgtactctgaaccccgccccattggcacccgtttcctcacccgtaatctgtaattgcctgttaatcaataaaccggttgattcgtttcagttgaactttggtctctgcgaagggcgaattcgtttaaacctgcaggactagaggtcctgtattagaggtcacgtgagtgttttgcgacattttgcgacaccatgtggtcacgctgggtatttaagcccgagtgagcacgcagggtctccattttgaagcgggaggtttgaacgcgcagccgccaagccgaattctgcagataaacagcgggcggaggggtggagtcgtgacgtgaattacgtcatagggttagggaggtcctgtattagaggtcacgtgagtgttttgcgacattttgcgacaccatgtggtctcgctgggggggggggcccgagtgagcacgcagggtctccattttgaagcgggaggtttgaacgagcgctggcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggaaattgtaagcgttaatattttgttaaaattcgcgttaaatttttgttaaatcagctcatttttttaaccaataggccgaaatcggcaaaatcccttataaatcaaaagaatagaccgagatagggttgagtgttgttccagtttggaacaagagtccactattaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaa